(19) United States
(12) Patent Application Publication
(10) Pub. No.: US 2025/0093334 A1
(43) Pub. Date: Mar. 20, 2025

(54) SYSTEMS AND METHODS FOR ASSAYING SECRETOME

(71) Applicant: Seer, Inc., Redwood City, CA (US)

(72) Inventors: Xiaoyan ZHAO, Foster City, CA (US); Ji Won HA, Redwood City, CA (US); Margaret DONOVAN, Livermore, CA (US); Daniel HORNBURG, Foster City, CA (US); Asim SIDDIQUI, San Francisco, CA (US)

(21) Appl. No.: 18/727,620

(22) PCT Filed: Jan. 13, 2023
(86) PCT No.: PCT/US2023/060635

§ 371 Date: Jul. 9, 2024

Related U.S. Application Data
(60) Provisional application No. 63/299,859, filed on Jan. 14, 2022.
(60) Provisional application No. 63/323,976, filed on Mar. 25, 2022.
(60) Provisional application No. 63/348,674, filed on Jun. 3, 2022.
(60) Provisional application No. 63/352,191, filed on Jun. 14, 2022.

(51) Int. Cl. G01N 33/50; C12M 1/00; C12M 1/26; C12M 1/34; G01N 33/68; G16B 40/10

(52) U.S. Cl. CPC G01N 33/5038; C12M 23/34; C12M 33/14; C12M 41/46; G01N 33/5076; G01N 33/6848; G16B 40/10

(57) ABSTRACT

In some aspects, the present disclosure provides a method for identifying one or more biomolecules. In some embodiments, the method comprises generating a set of biomolecules by incubating a cell in a supplemented medium under conditions sufficient for the cell to generate the set of biomolecules. In some embodiments, the method comprises contacting at least a portion of the supplemented medium with one or more surfaces to adsorb the set of biomolecules. In some embodiments, the method comprises removing the one or more surfaces and the set of biomolecules from the at least the portion of the supplemented medium to produce a separated sample. In some embodiments, the method comprises releasing, in the separated sample, the set of biomolecules from the one or more surfaces. In some embodiments, the method comprises detecting at least a subset of the set of biomolecules, thereby identifying one or more biomolecules.

### CROSS-REFERENCE

This application claims the benefit of U.S. Provisional Patent Application No. 63/352,191, filed on Jun. 14, 2022; U.S. Provisional Patent Application No. 63/348,674, filed on Jun. 3, 2022; U.S. Provisional Patent Application No. 63/323,976, filed on Mar. 25, 2022; and U.S. Provisional Patent Application No. 63/299,859, filed on Jan. 14, 2022; each of which applications is incorporated herein by reference in its entirety

### BACKGROUND

Conditioned media of different cell cultures can be used for a variety of in vitro biological applications. In some cases, the in vitro biological applications may include characterizing biomolecules, which are secreted from the cell cultures into the conditioned media, under different conditions. In some cases, the characterization of the secreted biomolecules from the in vitro biological applications may help develop an insight into an observed biological function.

### SUMMARY

In some aspects, the present disclosure provides a method for identifying one or more biomolecules, comprising: (a) providing a supplemented medium; (b) generating a set of biomolecules by incubating a cell in the supplemented medium under conditions sufficient for the cell to generate the set of biomolecules; (c) contacting at least a portion of the supplemented medium with one or more surfaces to adsorb the set of biomolecules; (d) removing the one or more surfaces and the set of biomolecules from the at least the portion of the supplemented medium to produce a separated sample; (e) releasing, in the separated sample, the set of biomolecules from the one or more surfaces; and (f) detecting at least a subset of the set of biomolecules, thereby identifying one or more biomolecules.

In some embodiments, subsequent to the contacting of (b), the set of biomolecules comprises a reduced dynamic range when adsorbed on the one or more surfaces compared to an original dynamic range of the set of biomolecules in the supplemented medium

In some embodiments, in (a), the incubating the cell in the supplemented medium is performed for less than about 5 seconds, 5 minutes, 5 hours, or 5 days.

In some embodiments, the cell is afflicted with an infection or a mutation.

In some embodiments, the cell is a viable cell comprising a cancer cell, an epithelial cell, a bone cell, a muscle cell, a fat cell, a tissue cell, a senescent cell, a pluripotent cell, a stem cell, or a nerve cell.

In some embodiments, the cell is a cancer cell that is a biopsied cell of a patient.

In some embodiments, a particle comprises the surface.

In some embodiments, the particle is a nanoparticle.

In some embodiments, the contacting in (b) further comprises contacting the biological sample with a second surface to adsorb a second plurality of biomolecules onto the second surface.

In some embodiments, the detecting comprises mass spectrometry.

In some embodiments, the conditions sufficient for the cell to generate the set of biomolecules with the supplemented medium comprises a predetermined temperature, a predetermined pressure, a predetermined flow regime, a predetermined solvent environment, or a combination thereof.

In some embodiments, the conditions are kept constant.

In some embodiments, the supplemented medium comprises as a fraction or in whole a serum, a plasma, cerebral spinal fluid (CSF), synovial fluid (SF), urine, tears, crevicular fluid, semen, whole blood, milk, nipple aspirate, needle aspirate, ductal lavage, vaginal fluid, nasal fluid, ear fluid, gastric fluid, pancreatic fluid, trabecular fluid, lung lavage, prostatic fluid, sputum, fecal matter, bronchial lavage, fluid from swabbings, bronchial aspirants, sweat, saliva, or any combination thereof.

In some embodiments, the supplemented medium comprises a supernatant of a cell culture, a secretome of co-cultures, an exosome, a tissue or cell lysate, or any combination thereof.

In some embodiments, the supplemented medium comprises a synthetic supplemented medium.

In some embodiments, the set of biomolecules comprise one or more biomarkers, molecular signatures, secreted proteins, absorbed proteins, a secretome, an exosome, or any combination thereof.

In some embodiments, the method further comprises repeating (a)-(e) for a second supplemented medium comprising a second set of biomolecules, wherein the second supplemented medium is generated by incubating the cell in the supplemented medium for a different length of time.

In some embodiments, the providing is performed by an automated fluidic system.

In some embodiments, the automated fluidic system comprises a microfluidic system.

In some embodiments, the automated fluidic system provides the supplemented medium at different times.

In some embodiments, the generating comprises an active secretion of at least a portion of the set of biomolecules.

In some embodiments, the generating comprises a passive release of at least a portion of the set of biomolecules.

In some embodiments, the generating comprises release of an exosome or liposome by the cell.

In some embodiments, the generating comprises apoptosis of the cell.

In some embodiments, the generating comprises necroptosis of the cell.

In some embodiments, a biomolecule of the set of biomolecules is a complex.

In some embodiments, a biomolecule of the set of biomolecules is a protein.

In some embodiments, a biomolecule of the set of biomolecules is a polypeptide.

In some embodiments, a biomolecule of the set of biomolecules is a nucleic acid.

In some embodiments, the cell is part of a plurality of cells.

In some embodiments, the plurality of cells are cells of a same type.

In some embodiments, the cell is of a tissue sample, an organoid, an immortalized cell line, or any combination thereof.

In some embodiments, the cell is a stem cell.

In some embodiments, the conditions sufficient for the cell to exchange the set of biomolecules with the supplemented medium comprises one or more of a presence or absence of an organic compound, a presence or absence of an inorganic compound, a presence or absence of an autocrine signaling molecule, a presence or absence of a paracrine signaling molecule, a presence or absence of an antigen, a presence or absence of one or more co-cultured cells, a presence or absence of radiation, a presence or absence of one or more toxins, a presence or absence of protein aggregates, a presence or absence of one or more proteins, a presence or absence of active viral particles, a presence or absence of inactivated viral particles, a presence or absence of applied heat or cooling, a presence or absence of applied mechanical stress, a presence or absence of electrical stimulation, a presence or absence of transposons, a presence or absence of exosomes, a presence or absence of liposomes, a presence or absence of coated nucleic acids, a presence or absence of shock, or any combination thereof.

In some embodiments, the conditions are varied over time.

In some embodiments, the contacting in (b) is performed such that the portion of the supplemented medium contacts one or more surface regions of the one or more surfaces, wherein the one or more surfaces regions do not comprise a specific targeting moiety.

In some embodiments, the method further comprises determining one or more protein to protein interactions, biomarkers, molecular signatures, biomolecules absorbed by the cell, biomolecules secreted by the cell, biomolecules dissociating from cell surfaces, biomolecules being cleaved off or shed from the surface, macromolecular complexes budded, released of cleaved of the surface, biomolecules passively released by the cell, conventionally and unconventionally released proteins, apoptotic release of biomolecules, necrotic released biomolecules, post-translation modifications, cell to cell interactions, cell to cell communications, or any combination thereof based at least in part on the identification, wherein molecular signatures are any patterns of proteins/proteoforms indicative of a biological state.

In some embodiments, the one or more biomolecules secreted by the cell are conventional secretions, unconventional secretions, type 1 unconventional secretions, or type 2 unconventional secretions.

In some embodiments, the set of biomolecules comprises a biomolecule assembly.

In some embodiments, the biomolecule assembly comprises quaternary protein, a vesicle, or an exosome.

In some embodiments, the cell is a eukaryote or a prokaryote.

In some embodiments, the method further comprises a plurality of cells comprising the cell.

In some embodiments, the plurality of cells is comprised in a tissue, an organoid, an organism, or a plurality of organisms.

In some embodiments, the plurality of cells comprises at least a first cell of a first type and a second cell of a second type, such that the first cell exchanges one or more biomolecules of the set of biomolecules with the second cell.

In some embodiments, the first cell and the second cell are co-cultured.

In some embodiments, the first cell is comprised in a feeder culture for the second cell. In some embodiments, the cell is derived from an immortalized cell line.

In some embodiments, the cell is a HeLa cell.

In some embodiments, the cell is a stem cell.

In some embodiments, the cell is comprised in a primary cell culture.

In some embodiments, the plurality of cells are disposed in a plurality of separate volumes, each volume comprising a different supplemented medium or incubating conditions. In some embodiments, the releasing comprises use of a protease.

In some embodiments, the one or more surfaces comprise at least two surfaces comprising distinct physicochemical properties such that the at least two surfaces adsorb a different pattern of biomolecule abundance from the set of biomolecules.

In some embodiments, the method further comprises determining the one or more biomolecules were generated by the cell and not originally present in the supplemented medium.

In some embodiments, the supplemented medium comprises fetal bovine serum.

In some aspects, the present disclosure provides a method for monitoring cell activity, comprising: (a) incubating a cell such that the cell generates a biological sample comprising a plurality of biomolecules; (b) contacting the biological sample with a surface to adsorb the plurality of biomolecules onto the surface; (c) separating the surface from the biological sample; (d) releasing at least a portion of the plurality of biomolecules on the surface; (e) detecting the at least the portion of the plurality of biomolecules, thereby identifying the plurality of biomolecules; and (f) repeating (a) through (d) after a predetermined amount of time, thereby monitoring an activity of the cell.

In some embodiments, the cell generates the biological sample by any one of producing, releasing, adsorbing, digesting, or modifying a biomolecule of the plurality of biomolecules.

In some embodiments, the method further comprises changing an incubation condition for the cell based at least in part on the activity of the cell.

In some embodiments, the incubating comprises exposing the cell to a supplemented medium, wherein the supplemented medium obscures detection of the at least the portion of the plurality of biomolecules.

In some embodiments, the method further comprises analyzing the activity of the cell.

In some embodiments, the at least the portion of the plurality of biomolecules comprise a dynamic range of at least about 6, 7, 8, 9, 10, 11, 12, 13, 14, 15, 16, 17, 18, 19, or 20.

In some embodiments, the at least the portion of the plurality of biomolecules comprise at least about 500, 600, 700, 800, 900, 1000, 2000, 3000, 4000, 5000, 6000, 7000, 8000, 9000, 10000, 20000, 30000, 40000, or 50000 biomolecules.

In some embodiments, (b) comprises contacting the biological sample with a plurality of surfaces.

In some embodiments, the releasing in (d) is performed using a protease that enzymatically cleaves the surface.

In some embodiments, the surface is disposed on a magnetic substrate, and wherein the separating in (c) is performed using a magnetic field to separate the magnetic substrate from the biological sample.

In some aspects, the present disclosure provides a method for identifying a low abundance biomolecule in a biological sample, comprising: (a) incubating a cell in a predetermined environment, such that the cell produces a biological sample; (b) contacting the biological sample with a surface to adsorb a plurality of low abundance biomolecules in the biological sample onto the surface; (c) separating the surface from the biological sample; (d) releasing at least a portion of the plurality of low abundance biomolecules on the surface; and (e) detecting the at least the portion of the plurality of low abundance biomolecules, thereby identifying the plurality of low abundance biomolecules.

In some embodiments, the method further comprises, before (c), adding a biomolecule to the biological sample, wherein the biomolecule reduces a detectability of the plurality of low abundance biomolecules in the biological sample.

In some embodiments, the detecting a low abundance biomolecule is about 7 orders of magnitude higher in signal than a signal from direct digestion of the cell in media.

In some embodiments, the incubating comprises exposing the cell to a supplemented medium, wherein the supplemented medium obscures detection of the at least the portion of the plurality of low abundance biomolecules.

In some embodiments, the plurality of low abundance biomolecules comprises at least about 500, 600, 700, 800, 900, 1000, 2000, 3000, 4000, 5000, 6000, 7000, 8000, 9000, 10000, 20000, 30000, 40000, or 50000 biomolecules.

In some embodiments, (b) comprises contacting the biological sample with a plurality of surfaces.

In some embodiments, the releasing in (d) is performed using a protease that enzymatically cleaves the surface.

In some embodiments, the surface is disposed on a magnetic substrate, and wherein the separating in (c) is performed using a magnetic field to separate the magnetic substrate from the biological sample.

In some aspects, the present disclosure provide an apparatus for assaying a biological sample, comprising: a substrate comprising a surface; a cell culture chamber comprising a cell; a loading unit that is operably coupled to the substrate and the cell culture chamber; and a computer readable medium comprising machine-executable code that, upon execution by a processor, implements a method comprising: (a) providing a controlled environment to the cell in the cell culture chamber for a predetermined duration of time, such that the cell produces a biological sample in the cell culture chamber; (b) transferring at least a portion of the biological sample from the cell culture chamber to the substrate using the loading unit, thereby contacting the at least the portion with the surface to adsorb a biomolecule in the at least the portion of the biological sample onto the surface; and (c) assaying at least a portion of the biomolecule to detect the biomolecule in the biological sample.

In some embodiments, the apparatus comprises a plurality of cell culture chambers.

In some embodiments, a first cell culture chamber in the plurality of cell culture chambers is provided with a first controlled environment and a second cell culture chamber in the plurality of cell culture chambers is provided with a second controlled environment.

In some embodiments, the transferring is performed using one or more fluidic connections and one or more pumps comprised within the loading unit.

In some embodiments, the transferring is performed using one or more pipettes and one or more pumps comprised within the loading unit.

In some aspects, the present disclosure provides a method for identifying one or more biomolecules, comprising: (a) incubating a cell under conditions sufficient for the cell to generate an exosome, wherein the exosome comprises a plurality of biomolecules; (b) contacting the exosome with one or more surfaces to capture at least a portion of the exosome; (c) removing the one or more surfaces and the at least the portion of the exosome from the cell to produce a separated sample; (d) releasing, in the separated sample, the at least the portion of the exosome from the one or more surfaces; and (e) detecting at least a subset of the plurality of biomolecules in the at least the portion of the exosome, thereby identifying one or more biomolecules.

In some aspects, the present disclosure provides a method for monitoring cell activity, comprising: (a) incubating a cell such that the cell generates a biological sample comprising an exosome, wherein the exosome comprises a plurality of biomolecules; (b) contacting the biological sample with a surface to capture at least a portion of the exosome onto the surface; (c) releasing the at least the portion of the exosome from the surface; (d) detecting the at least the portion of the plurality of biomolecules in the at least the portion of the exosome, thereby identifying the plurality of biomolecules; and (e) repeating (a) through (d) after a predetermined amount of time, thereby monitoring an activity of the cell.

In some aspects, the present disclosure provides a method for identifying a low abundance biomolecule in a biological sample, comprising: (a) incubating a cell in a predetermined environment, such that the cell produces a biological sample comprising an exosome, wherein the exosome comprises a plurality of low-abundance biomolecules; (b) contacting the biological sample with a surface to capture at least a portion of the exosome in the biological sample onto the surface; (c) releasing the at least the portion of the exosome from the surface; and (d) detecting at least the portion of the plurality of low abundance biomolecules in the at least the portion of the exosome, thereby identifying the plurality of low abundance biomolecules.

In some aspects, the present disclosure provides an apparatus for assaying a biological sample, comprising: a substrate comprising a surface; a cell culture chamber comprising a cell; a loading unit that is operably coupled to the substrate and the cell culture chamber; and a computer readable medium comprising machine-executable code that, upon execution by a processor, implements a method comprising: (a) providing a controlled environment to the cell in the cell culture chamber for a predetermined duration of time, such that the cell produces a biological sample comprising an exosome in the cell culture chamber, wherein the exosome comprises one or more biomolecules; (b) transferring at least a portion of the biological sample from the cell culture chamber to the substrate using the loading unit, thereby contacting the at least the portion with the surface to adsorb the exosome in the at least the portion of the biological sample onto the surface; and (c) assaying the exosome to detect the one or more biomolecules in the biological sample.

In some aspects, the present disclosure provides a method of identifying biomolecules, comprising: (a) processing one or more exosomes to release a plurality of biomolecules in the one or more exosomes to an environment external to the one or more exosomes, wherein a subset of biomolecules in the plurality of biomolecules comprises a first distribution of relative abundances in the one or more exosomes; (b) performing a composition improving assay on the plurality of biomolecules to increase the first distribution to a second distribution of relative abundances for the subset of biomolecules; and (c) assaying the plurality of biomolecules to identify the subset of biomolecules.

In some embodiments, the processing is performed at least partially by adding a lyse buffer to the one or more exosomes.

In some embodiments, the processing is performed at least partially by providing acoustic energy to the one or more exosomes.

In some embodiments, the processing is performed at least partially by freezing and thawing the one or more exosomes.

In some embodiments, the processing is performed at least partially by heating the one or more exosomes.

In some embodiments, the processing is performed at least partially by shearing the one or more exosomes.

In some embodiments, the processing is performed at least partially by grinding the one or more exosomes.

In some embodiments, the subset of biomolecules comprises at least about 100, 200, 300, 400, 500, 600, 700, 800, 900, 1,000, 2,000, 3,000, 4,000, 5,000, 6,000, 7,000, 8,000, 9,000, 10,000, 20,000, 30,000, 30,000, 40,000, 50,000, 60,000, 70,000, 80,000, 90,000, or 100,000 unique biomolecules.

In some embodiments, the composition improving assay comprises contacting the plurality of biomolecules with one or more surfaces to adsorb the subset of biomolecules on the one or more surfaces, such that the subset of biomolecules comprises the second distribution when the subset of biomolecules is adsorbed on the one or more surfaces.

In some embodiments, the contacting increases a visibility of the subset of biomolecules in the assaying step of (c) when the subset of biomolecules are adsorbed on the one or more surfaces, compared to the visibility of the subset of biomolecules when the subset of biomolecules are in the one or more exosomes, wherein the subset of biomolecules are low-abundance biomolecules comprising less than about 1 percent by mass of the plurality of biomolecules in the one or more exosomes.

In some aspects, the present disclosure provides a method of identifying biomolecules, comprising: (a) contacting a biological sample comprising one or more exosomes with a plurality of particles to non-specifically bind a subset of the one or more exosomes on the plurality of particles, wherein the plurality of particles comprises distinct physicochemical properties, and wherein the one or more exosomes comprise a plurality of biomolecules; (b) processing the one or more exosomes to release the plurality of biomolecules to an environment external to the one or more exosomes; and (c) assaying the plurality of biomolecules to identify at least a subset of biomolecules in the plurality of biomolecules.

In some embodiments, the processing is performed at least partially by adding a lyse buffer to the one or more exosomes.

In some embodiments, the processing is performed at least partially by providing acoustic energy to the one or more exosomes.

In some embodiments, the processing is performed at least partially by freezing and thawing the one or more exosomes.

In some embodiments, the processing is performed at least partially by heating the one or more exosomes.

In some embodiments, the processing is performed at least partially by shearing the one or more exosomes.

In some embodiments, the processing is performed at least partially by grinding the one or more exosomes.

In some embodiments, the subset of biomolecules comprises at least about 100, 200, 300, 400, 500, 600, 700, 800, 900, 1,000, 2,000, 3,000, 4,000, 5,000, 6,000, 7,000, 8,000, 9,000, 10,000, 20,000, 30,000, 30,000, 40,000, 50,000, 60,000, 70,000, 80,000, 90,000, or 100,000 unique biomolecules.

In some embodiments, the plurality of particles increases a visibility of the subset of biomolecules in the assaying step of (c) when the subset of biomolecules are adsorbed on the plurality of particles, compared to the visibility of the subset of biomolecules when the subset of biomolecules are in the one or more exosomes, wherein the subset of biomolecules are low-abundance biomolecules comprising less than about 1 percent by mass of the plurality of biomolecules in the one or more exosomes.

In some aspects, the present disclosure provides a method for characterizing a biological preparation, comprising: contacting the biological preparation with one or more surfaces to adsorb a plurality of biomolecules in the biological preparation onto the one or more surfaces, wherein the plurality of biomolecules comprises a product biomolecule and an impurity; and assaying the plurality of biomolecules to determine a deviation between a composition of the plurality of biomolecules and a reference composition, wherein the deviation is indicative of a purity or an activity of the biological preparation.

In some embodiments, the biological preparation comprises a cell culture.

In some embodiments, the plurality of biomolecules comprises a secretome or an exosome from the cell culture.

In some embodiments, the plurality of biomolecules comprises a portion of one or more cells of the cell culture.

In some embodiments, the cell culture comprises a host organism that produces the product biomolecule.

In some embodiments, the cell culture comprises a host organism that produces the impurity.

In some embodiments, the cell culture comprises a contaminating organism that produces the impurity.

In some embodiments, the impurity comprises a host-cell protein.

In some embodiments, the host-cell protein comprises a protease, a lipase, or an isomerase.

In some embodiments, the host-cell protein comprises a passively released protein or an actively released protein.

In some embodiments, the host-cell protein comprises a part of the host organism.

In some embodiments, the part comprises a cell membrane, an organelle, or both

In some embodiments, the cell culture comprises a plurality of host organism species or strains.

In some embodiments, the cell culture comprises a plurality of contaminating species or strains.

In some embodiments, the biological preparation comprises a plurality of impurities.

In some embodiments, the biological preparation comprises a plurality of product biomolecules.

In some embodiments, the host organism comprises a prokaryotic host organism or a eukaryotic host organism.

In some embodiments, the prokaryotic host organism comprises Escherichia coli, Streptomyces sp., acetic acid bacteria, lactic acid bacteria, a thermophilic Bacillus sp., Clostridium thermocellus, Agrobacterium tumefaciens, Thermus aquaticus, Bacillus coagulans, Pseudomonas stutzeri, Acetobacter sp., Micrococcus sp., Haemophilus influenzae, or Leuconostoc mesenteroides.

In some embodiments, the eukaryotic host organism comprises a yeast, a fungus, a HeLa cell, a stem cell, a cancer cell, a genetically modified cell, or an algae.

In some embodiments, the genetically modified cell comprises an exogenous nucleic acid sequence that encodes the product biomolecule.

In some embodiments, the impurity is a proteoform of a biomolecule in the plurality of biomolecules.

In some embodiments, the impurity is a proteoform of the product biomolecule.

In some embodiments, the proteoform comprises a splicing variant, an allelic variant, or a post-translational modification variant.

In some embodiments, the post-translational modification variant comprises a post-translational modification comprising: acylation, alkylation, prenylation, flavination, amination, deamination, carboxylation, decarboxylation, nitrosylation, halogenation, sulfurylation, glutathionylation, oxidation, oxygenation, reduction, ubiquitination, SUMOylation, neddylation, myristoylation, palmitoylation, isoprenylation, farnesylation, geranylgeranylation, glypiation, glycosylphosphatidylinositol anchor formation, lipoylation, heme functionalization, phosphorylation, phosphopantetheinylation, retinylidene Schiff base formation, diphthamide formation, ethanolamine phosphoglycerol functionalization, hypusine formation, beta-Lysine addition, acetylation, formylation, methylation, amidation, amide bond formation, butyrylation, gamma-carboxylation, glycosylation, polysialylation, malonylation, hydroxylation, iodination, nucleotide addition, phosphate ester formation, phosphoramidate formation, adenylation, uridylylation, propionylation, pyroglutamate formation, gluthathionylation, sulfenylation, sulfinylation, sulfonylation, succinylation, sulfation, glycation, carbonylation, isopeptide bond formation, biotinylation, carbamylation, oxidation, pegylation, citrullination, deamidation, eliminylation, disulfide bond formation, proteolytic cleavage, isoaspartate formation, racemization, protein splicing, chaperon-assisted folding, or any combination thereof.

In some embodiments, a difference between a first log (water-octanol partition coefficient) the impurity and a second log (water-octanol partition coefficient) the product biomolecule is less than about 2, 1.5, 1, 0.9, 0.8, 0.7, 0.6, 0.5, 0.4, 0.3, 0.2, 0.1, 0.09, 0.08, 0.07, 0.06, 0.05, 0.04, 0.03, 0.02, or 0.01.

In some embodiments, a difference between a first log (water-octanol partition coefficient) the impurity and a second log (water-octanol partition coefficient) the product biomolecule is greater than about 2, 1.5, 1, 0.9, 0.8, 0.7, 0.6, 0.5, 0.4, 0.3, 0.2, 0.1, 0.09, 0.08, 0.07, 0.06, 0.05, 0.04, 0.03, 0.02, or 0.01.

In some embodiments, the impurity and the product biomolecule are hydrophobic.

In some embodiments, the impurity and the product biomolecule are hydrophilic.

In some embodiments, a difference between a first pKa of the impurity and a second pKa of the product biomolecule is at most 3, 2, 1, 0.9, 0.8, 0.7, 0.6, 0.5, 0.4, 0.3, 0.2, 0.1, 0.09, 0.08, 0.07, 0.06, 0.05, 0.04, 0.03, 0.02, or 0.01.

In some embodiments, a difference between a first pKa of the impurity and a second pKa of the product biomolecule is at least 3, 2, 1, 0.9, 0.8, 0.7, 0.6, 0.5, 0.4, 0.3, 0.2, 0.1, 0.09, 0.08, 0.07, 0.06, 0.05, 0.04, 0.03, 0.02, or 0.01.

In some embodiments, the impurity and the product biomolecule are stereoisomers of one another.

In some embodiments, the impurity and the product biomolecule comprise enantiomers of one another.

In some embodiments, a difference between a first mass-to-charge ratio of the impurity and a second mass-to-charge ratio of the product biomolecule is at most 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, or 100 kiloDaltons/e−.

In some embodiments, a difference between a first mass-to-charge ratio of the impurity and a second mass-to-charge ratio of the product biomolecule is at least 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, or 100 kiloDaltons/e−.

In some embodiments, the impurity comprises a first histidine tag and the product biomolecule comprises a second histidine tag.

In some embodiments, the first histidine tag and the second histidine tag comprises the same number of amino acids.

In some embodiments, a difference between a first molecular weight of the impurity and a second molecular weight of the product biomolecule is at most 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, or 100 kiloDaltons.

In some embodiments, a difference between a first molecular weight of the impurity and a second molecular weight of the product biomolecule is at least 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, or 100 kiloDaltons.

In some embodiments, the method further comprises purifying an impure biological preparation before (a) to generate the biological preparation, wherein the impure biological preparation and the biological preparation comprises the impurity.

In some embodiments, the purifying increases the purity or the activity of the biological preparation compared to the impure biological preparation.

In some embodiments, the impurity comprises a lower abundance than the product biomolecule.

In some embodiments, the impurity is at least 1, 2, 3, 4, 5, 6, 7, 8, 9, 10, 11, 12, 13, 14 or 15 times in magnitude less abundant than the product biomolecule by count, mass, or mass spectrometry signal intensity.

In some embodiments, the impurity is at most 1, 2, 3, 4, 5, 6, 7, 8, 9, 10, 11, 12, 13, 14 or 15 times in magnitude less abundant than the product biomolecule by count, mass, or mass spectrometry signal intensity.

In some embodiments, the plurality of biomolecules comprises a reduced dynamic range on the one or more surfaces compared to a dynamic range of the plurality of biomolecule in the biological preparation.

In some embodiments, the reduced dynamic range is at least 1, 2, 3, 4, 5, 6, 7, 8, 9, 10, 11, 12, 13, 14 or 15 times in magnitude less than the dynamic range.

In some embodiments, the reduced dynamic range is at most 1, 2, 3, 4, 5, 6, 7, 8, 9, 10, 11, 12, 13, 14 or 15 times in magnitude less than the dynamic range.

In some embodiments, the biological preparation comprises a drug or a metabolite thereof.

In some embodiments, the impurity comprises the drug.

In some embodiments, the impurity comprises the metabolite thereof.

In some embodiments, the product biomolecule comprises the drug.

In some embodiments, the host cell protein comprises the drug.

In some embodiments, the drug comprises an antibody, a biotherapeutic, or a chemotherapeutic biomolecule.

In some embodiments, the drug comprises a methamphetamine, isotretinoin, an antibiotic, an anti-platelet medication, dutasteride, a blood thinner, insulin, hepatitis B immune globulin, a growth hormone, tamsulosin, finasteride, acitretin, etretinate, or any combination thereof.

In some embodiments, the impurity is capable of reducing the activity of the product biomolecule, wherein the impurity is produced by the host organism or a contaminating organism that is extraneous to the host organism.

In some embodiments, the biological preparation comprises a vaccine.

In some embodiments, the product biomolecule comprises an antigen of a pathogenic bacteria, a virus, or a derivative thereof.

In some embodiments, the product biomolecule comprises an immunoprotein or a derivative thereof.

In some embodiments, the product biomolecule comprises an enzyme.

In some embodiments, the enzyme is configured to degrade a synthetic polymer, degrade an oil, or catalyze ethanol production.

In some embodiments, the biological preparation comprises blood, plasma, platelets, clotting factors, or any combination thereof.

In some embodiments, the impurity comprises a biomolecule produced by a pathogen.

In some embodiments, the pathogen comprises a Hepatitis B virus, a Hepatitis C virus, a COVID-19, or a HIV.

In some embodiments, the biological preparation comprises a human consumable product or a livestock consumable product.

In some embodiments, the human consumable product or the livestock consumable product comprises poultry, beef, pork, vegetables, fungi, a fermented product, or a meat substitute.

In some embodiments, the impurity comprises a bacterial biomolecule.

In some embodiments, the method further comprises estimating a shelf life of the biological preparation based on the deviation.

In some embodiments, the method further comprises determining a fitness of the biological preparation for human or livestock consumption based on the deviation.

In some embodiments, the human consumable product comprises a fermented product.

In some embodiments, the fermented product comprises ethanol, acetic acid, or lactic acid.

In some embodiments, the fermented product comprises beer or wine.

In some embodiments, the fermented product comprises vinegar.

In some embodiments, the fermented product comprises yogurt.

In some embodiments, the impurity is capable of increasing the risk of complications when the biological preparation is administered to a subject.

In some embodiments, the impurity, when administered to a human subject or ingested by the human subject, is expected to reduce the activity/stability of the product biomolecule on the human subject.

In some embodiments, the impurity, when administered to a human subject or ingested by the human subject, is capable of harming the human subject

In some embodiments, the biological preparation, when comprising the host organism or the contaminating organism and administered to a human subject or ingested by the human subject, is capable of harming the human subject.

In some embodiments, the harming comprises: an immune response, a blood coagulation, an allergic reaction, a food poisoning, or any combination thereof.

In some embodiments, the deviation comprises a difference between a level of activity of the biological preparation and a reference level of activity for the reference composition.

In some embodiments, the level of activity is assayed by contacting the plurality of biomolecules on the one or more surfaces with an activity assay.

In some embodiments, the activity assay comprises an in vitro cell culture.

In some embodiments, the activity assay comprises a substrate.

In some embodiments, the activity assay comprises an immunoaffinity assay.

In some embodiments, the activity assay comprises an avidity assay.

In some embodiments, the deviation comprises a difference between a level of safety of the biological preparation and a reference level of safety for the reference composition.

In some embodiments, the level of safety is assayed by contacting the plurality of biomolecules on the one or more surfaces with a safety assay.

In some embodiments, the level of safety comprises a level of immunogenicity and the reference level of safety comprises a reference level of immunogenicity.

In some embodiments, the level of immunogenicity is assayed by contacting the plurality of biomolecules on the one or more surfaces with one or more human blood samples or derivatives thereof.

In some embodiments, the one or more human blood samples or derivatives thereof comprises one or more white blood cells.

In some embodiments, the deviation comprises a detectable level of the impurity.

In some embodiments, the deviation comprises a level of the product biomolecule that is below a reference level of the product biomolecule.

In some embodiments, the deviation comprises a difference between a level of the impurity and a reference level of the impurity.

In some embodiments, the method further comprises purifying the biological preparation based on the deviation.

In some embodiments, the purifying is performed when the difference is greater than 1, 2, 3, 4, 5, or 6 times a standard deviation of measurement for the level of the impurity.

In some embodiments, the purifying is performed when the difference is greater than 1, 2, 3, 4, 5, or 6 times a standard error of measurement for the level of the impurity, wherein the standard error is based on at least N number of assays.

In some embodiments, the reference composition comprises at least about 1, 2, 3, 4, 5, 6, 7, 8, 9, 10, 20, 30, 40, 50, 60, 70, 80, 90, 91, 92, 93, 94, 95, 96, 97, 98, 99, 99.1, 99.2, 99.3, 99.4, 99.5, 99.6, 99.7, 99.8, 99.9, 99.9, 99.91, 99.92, 99.93, 99.94, 99.95, 99.96, 99.97, 99.98, 99.99, 99.99, 99.991, 99.992, 99.992, 99.993, 99.994, 99.995, 99.996, 99.997, 99.998, 99.999, 99.999, 99.9991, 99.9992, 99.9993, 99.9994, 99.9995, 99.9996, 99.9997, 99.9998, or 99.9999 percent purity of the product biomolecule.

In some embodiments, the reference composition comprises at most about 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 91, 92, 93, 94, 95, 96, 97, 98, 99, 99.1, 99.2, 99.3, 99.4, 99.5, 99.6, 99.7, 99.8, 99.9, 99.9 percent purity of the impurity.

In some embodiments, the reference composition comprises at most about 1, 2, 3, 4, 5, 6, 7, 8, 9, 10, 20, 30, 40, 50, 60, 70, 80, 90, 100, 200, 300, 400, 500, 600, 700, 800, or 900 ppm of the impurity.

In some embodiments, the reference composition comprises at most about 1, 2, 3, 4, 5, 6, 7, 8, 9, 10, 20, 30, 40, 50, 60, 70, 80, 90, 100, 200, 300, 400, 500, 600, 700, 800, or 900 ppb of the impurity.

In some embodiments, the percent purity is based on a signal intensity of the product determined from the assaying.

In some embodiments, the deviation is determined using a machine learning algorithm.

In some embodiments, the machine learning algorithm is configured to receive one or more features that represent composition of the plurality of biomolecules and output the deviation based on the composition.

In some embodiments, the machine learning algorithm is trained to learn the reference level of the impurity based on a first plurality of samples comprising the purity or the activity above a predetermined threshold, a second plurality of samples comprising the purity or the activity below the predetermined threshold, or both.

In some embodiments, the method further comprises, using the machine learning algorithm, classifying the biological preparation as comprising the purity or the activity above the predetermined threshold or below the predetermined threshold based on the composition of the plurality of biomolecule.

In some embodiments, the method further comprises monitoring the biological preparation by assaying the plurality of biomolecules a plurality of times to determine a plurality of deviation at the plurality of times.

In some embodiments, the method further comprises determining a drift in the genetic makeup of the host organism based on the plurality of deviations.

In some embodiments, the method further comprises determining a drift in the population of the cell culture based on the plurality of deviations.

In some embodiments, the method further comprises determining a contamination in a workflow for producing the biological preparation based on the plurality of deviations.

In some aspects, the present disclosure provides a method for detecting an impurity in a biological preparation, comprising: contacting the biological preparation with the one or more surfaces to adsorb a plurality of biomolecules in the biological preparation onto the one or more surfaces; and assaying the plurality of biomolecules to detect a biomolecule fingerprint of the biological preparation, wherein the biomolecule fingerprint comprises a signature of the impurity in the plurality of biomolecules.

In some aspects, the present disclosure provides an apparatus for characterizing biological preparation, comprising: a first chamber configured to hold the biological preparation, wherein the biological preparation comprises a plurality of biomolecules, wherein the plurality of biomolecules comprises a product biomolecule and an impurity; a second chamber comprising one or more surfaces; a loader operably coupled to the first chamber and the second chamber, wherein the loader is configured to transfer the biological preparation between the first chamber and the second chamber; and a computer readable medium for measuring a purity or an activity of the biological preparation comprising machine-executable code that, upon execution by a processor, implements a method comprising: contacting, using the loader, the biological preparation from the first chamber with the one or more surfaces in the second chamber to adsorb the plurality of biomolecules in the biological preparation onto the one or more surfaces.

In some embodiments, the method further comprises assaying the plurality of biomolecules to determine a deviation between a composition of the plurality of biomolecules and a reference composition, wherein the deviation indicates the purity or the activity of the biological preparation.

In some embodiments, apparatus further comprises a first separator operably coupled to the second chamber, wherein the first separator is configured to separate the plurality of biomolecules adsorbed on the one or more surfaces from the biological preparation.

In some embodiments, the first separator comprises a magnet.

In some embodiments, the apparatus further comprises a second separator operably coupled to the second chamber, wherein the second separator is configured to separate the plurality of biomolecules adsorbed on the one or more surfaces from the one or more surfaces.

In some embodiments, the apparatus further comprises one or more purifiers for purifying the biological preparation.

In some embodiments, the method further comprises purifying the biological preparation using the one or more purifiers based on the product quality.

In some embodiments, the one or more purifiers comprise a plurality of purifiers.

In some embodiments, the plurality of purifiers is provided in a sequence such that a purified biological preparation from one purifier in the plurality of purifiers feeds into another purifier in the plurality of purifiers.

In some embodiments, process conditions of the plurality of purifiers are based on the product quality.

In some embodiments, the method further comprises using the one or more purifiers to purify the biological preparation based on the deviation to improve the purity or the activity of the biological preparation.

In some embodiments, the apparatus further comprises a third chamber operably coupled to the loader, wherein the chamber comprises an in vitro cell culture or human blood samples or derivatives thereof.

In some embodiments, the method further comprises certifying or rejecting the biological preparation based on the deviation.

In some embodiments, the apparatus further comprises a mass spectrometer for performing the assaying to determine the deviation.

In some embodiments, the first chamber comprises an incubator.

In some embodiments, the first chamber comprises a heater or a cooler operably connected to the incubator.

In some embodiments, the method comprises controlling the temperature of the first chamber using the heater or the cooler.

In some embodiments, the first chamber is pressurized

In some embodiments, the apparatus comprises a plurality of chamber comprising the first chamber, wherein each chamber in the plurality of chambers is operably connected to the loader and the second chamber, and wherein each chamber in the plurality of chambers comprises a portion of the biological preparation.

In some embodiments, each chamber in the plurality of chambers comprises a different species or strain of a host organism configured to produce at least a portion of the biological preparation.

In some embodiments, the apparatus further comprises one or more particles comprising the one or more surfaces and one or more supports.

In some embodiments, the one or more supports comprise a paramagnetic material.

In some embodiments, the paramagnetic material comprises a superparamagnetic material.

In some embodiments, the one or more surfaces comprises a plurality of surface types.

In some embodiments, the one or more particles comprise a plurality of particles comprising the plurality of surface types.

In some embodiments, the one or more particles comprise one or more microparticles.

In some embodiments, the one or more particles comprise one or more nanoparticles.

In some embodiments, the one or more particles comprise one or more porous particles.

In some embodiments, the one or more surfaces are configured to reduce a dynamic range of the plurality of biomolecules in the biological preparation when the plurality of biomolecules is adsorbed on the one or more surfaces.

In some embodiments, the impurity comprises a lower abundance than the product biomolecule.

In some embodiments, the impurity is at least 1, 2, 3, 4, 5, 6, 7, 8, 9, 10, 11, 12, 13, 14 or 15 times in magnitude less abundant than the product biomolecule by count, mass, or mass spectrometry signal intensity.

In some embodiments, the impurity is at most 1, 2, 3, 4, 5, 6, 7, 8, 9, 10, 11, 12, 13, 14 or 15 times in magnitude less abundant than the product biomolecule by count, mass, or mass spectrometry signal intensity.

In some embodiments, the dynamic range of the plurality of biomolecules is reduced by at least 1, 2, 3, 4, 5, 6, 7, 8, 9, 10, 11, 12, 13, 14 or 15 times in magnitude.

In some embodiments, the reduced dynamic range is at most 1, 2, 3, 4, 5, 6, 7, 8, 9, 10, 11, 12, 13, 14 or 15 times in magnitude less than the dynamic range.

In some aspects, the present disclosure provides an apparatus for detecting an impurity in a biological preparation, comprising: a plurality of chambers in operable connection with one another, wherein the plurality of chambers comprises one or more surfaces; one or more fluid transfer devices operably coupled to the plurality of chambers; and a computer readable medium for detecting the impurity in the biological preparation comprising machine-executable code that, upon execution by a processor, implements a method comprising: contacting, using the one or more fluid transfer devices, the biological preparation with the one or more surfaces to adsorb a plurality of biomolecules in the biological preparation onto the one or more surfaces.

In some embodiments, the method further comprises assaying the plurality of biomolecules to detect a biomolecule fingerprint of the biological preparation, wherein the biomolecule fingerprint comprises a signature of the contaminating biomolecule in the plurality of biomolecules.

In some embodiments, the apparatus further comprises a first separator operably coupled to the plurality of chambers, wherein the first separator is configured to separate the plurality of biomolecules adsorbed on the one or more surfaces from the biological preparation.

In some embodiments, the first separator comprises a magnet.

In some embodiments, the apparatus further comprises a second separator operably coupled to the second chamber, wherein the second separator is configured to separate the plurality of biomolecules adsorbed on the one or more surfaces from the one or more surfaces.

In some embodiments, the apparatus further comprises one or more purifiers for purifying the biological preparation.

In some embodiments, the method further comprises purifying the biological preparation using the one or more purifiers based on the biomolecule fingerprint.

In some embodiments, the one or more purifiers comprise a plurality of purifiers.

In some embodiments, the plurality of purifiers is provided in a sequence such that a purified biological preparation from one purifier in the plurality of purifiers feeds into another purifier in the plurality of purifiers.

In some embodiments, process conditions of the plurality of purifiers are based on the biomolecule fingerprint.

In some embodiments, the method further comprises using the one or more purifiers to purify the biological preparation based on the biomolecule fingerprint to reduce the signature of the contaminating biomolecule.

In some embodiments, the apparatus further comprises a third chamber operably coupled to the loader, wherein the chamber comprises an in vitro cell culture or human blood samples or derivatives thereof.

In some embodiments, the method further comprises certifying or rejecting the biological preparation based on the biomolecule fingerprint.

In some embodiments, the apparatus further comprises a mass spectrometer for performing the assaying to determine the biomolecule fingerprint.

In some embodiments, the plurality of chambers comprises an incubator.

In some embodiments, the plurality of chambers comprises a heater or a cooler operably connected to the incubator.

In some embodiments, the method comprises controlling the temperature the plurality of chambers using the heater or the cooler.

In some embodiments, the plurality of chambers is pressurized

In some embodiments, each chamber in the plurality of chambers comprises a different species or strain of a host organism configured to produce at least a portion of the biological preparation.

In some embodiments, the apparatus further comprises one or more particles comprising the one or more surfaces and one or more supports.

In some embodiments, the one or more supports comprise a paramagnetic material.

In some embodiments, the paramagnetic material comprises a superparamagnetic material.

In some embodiments, the one or more surfaces comprises a plurality of surface types.

In some embodiments, the one or more particles comprise a plurality of particles comprising the plurality of surface types.

In some embodiments, the one or more particles comprise one or more microparticles.

In some embodiments, the one or more particles comprise one or more nanoparticles.

In some embodiments, the one or more particles comprise one or more porous particles.

In some embodiments, the one or more surfaces are configured to reduce a dynamic range of the plurality of biomolecules in the biological preparation when the plurality of biomolecules is adsorbed on the one or more surfaces.

In some embodiments, the contaminating biomolecule is at least 1, 2, 3, 4, 5, 6, 7, 8, 9, 10, 11, 12, 13, 14 or 15 times in magnitude less abundant than another biomolecule in the plurality of biomolecules by count, mass, or mass spectrometry signal intensity.

In some embodiments, the contaminating biomolecule is at most 1, 2, 3, 4, 5, 6, 7, 8, 9, 10, 11, 12, 13, 14 or 15 times in magnitude less abundant than another biomolecule in the plurality of biomolecules by count, mass, or mass spectrometry signal intensity.

In some embodiments, the dynamic range of the plurality of biomolecules is reduced by at least 1, 2, 3, 4, 5, 6, 7, 8, 9, 10, 11, 12, 13, 14 or 15 times in magnitude.

In some aspects, the present disclosure provides a computer-implemented method for generating a quality metric for a biological preparation, comprising: receiving a plurality of mass spectrometry datasets for a plurality of polyamino acids in the biological preparation, wherein the plurality of polyamino acids comprises at least one product biomolecule and a plurality of impurities; generating a plurality of polyamino acid identifications and a plurality of polyamino acid abundances for the plurality of polyamino acids based on the plurality of mass spectrometry datasets; and processing the plurality of polyamino acid identifications to output the quality metric for the biological preparation.

In some embodiments, the plurality of polyamino acid abundances is indicative of relative abundances between the plurality of polyamino acids

In some embodiments, the plurality of polyamino acid identifications comprise at least 1000, 2000, 3000, 4000, 5000, 6000, 7000, 8000, 9000, 10000, 15000, 20000, 25000, 30000, 35000, 40000, 45000, or 50000 polyamino acid identifications.

In some embodiments, the plurality of polyamino acids comprises a dynamic range of at least about 6, 7, 8, 9, 10, 11, 12, 13, 14, or 15 in the biological preparation.

In some embodiments, the plurality of polyamino acid abundances comprises a dynamic range less than about 6, 7, 8, 9, 10, 11, 12, 13, 14, or 15.

In some embodiments, the plurality of impurities comprises a proteoform of a polyamino acid in the plurality of polyamino acids.

In some embodiments, the proteoform comprises a splicing variant, an allelic variant, or a post-translational modification variant.

In some embodiments, the post-translational modification variant comprises a post-translational modification comprising: acylation, alkylation, prenylation, flavination, amination, deamination, carboxylation, decarboxylation, nitrosylation, halogenation, sulfurylation, glutathionylation, oxidation, oxygenation, reduction, ubiquitination, SUMOylation, neddylation, myristoylation, palmitoylation, isoprenylation, farnesylation, geranylgeranylation, glypiation, glycosylphosphatidylinositol anchor formation, lipoylation, heme functionalization, phosphorylation, phosphopantetheinylation, retinylidene Schiff base formation, diphthamide formation, ethanolamine phosphoglycerol functionalization, hypusine formation, beta-Lysine addition, acetylation, formylation, methylation, amidation, amide bond formation, butyrylation, gamma-carboxylation, glycosylation, polysialylation, malonylation, hydroxylation, iodination, nucleotide addition, phosphate ester formation, phosphoramidate formation, adenylation, uridylylation, propionylation, pyroglutamate formation, gluthathionylation, sulfenylation, sulfinylation, sulfonylation, succinylation, sulfation, glycation, carbonylation, isopeptide bond formation, biotinylation, carbamylation, oxidation, pegylation, citrullination, deamidation, eliminylation, disulfide bond formation, proteolytic cleavage, isoaspartate formation, racemization, protein splicing, chaperon-assisted folding, or any combination thereof.

In some embodiments, the processing comprises using a machine learning algorithm to score the biological preparation based on the plurality of polyamino acid identifications and the plurality of polyamino acid abundances to generate the quality metric.

In some embodiments, the machine learning algorithm is trained using a first set of samples above a predetermined quality metric threshold, a second set of samples below the determined quality metric threshold, or both.

In some embodiments, the processing is based on a difference between the plurality of polyamino acid identifications and the plurality of polyamino acid abundances and a reference plurality of polyamino acid identifications and a reference plurality of polyamino acid abundances.

In some embodiments, the quality metric comprises a purity metric, an activity metric, a safety metric, or any combination thereof.

In some aspects, the present disclosure provides a computer-implemented system comprising: a digital processing device comprising: at least one processor, an operating system configured to perform executable instructions, a memory, and a computer program including instructions executable by the digital processing device to: receive a plurality of mass spectrometry datasets obtained from a plurality of biological preparations; processing the plurality of mass spectrometry datasets in real-time to generate a plurality of quality metrics for the plurality of biological preparations; and providing process control instructions to a manufacturing process for producing the plurality of biological preparations.

In some embodiments, the process control instructions comprise certifying or rejecting one or more biological preparations in the plurality of biological preparations.

In some embodiments, the process control instructions comprise providing change one or more process conditions of the manufacturing process.

In some embodiments, the processing is performed using one or more cloud-computing nodes.

In some embodiments, the processing is performed in less than 1, 2, 3, 4, 5, 6, 7, 8, 9, 10, 15, 30, 60, 120, 180, 240, 300, or 360 minutes.

In some embodiments, the plurality of biological preparations comprises a plurality of impurities.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIGS. 1A-1E illustrate methods for assaying a secretome using a composition improving assay, in accordance with some embodiments.

FIGS. 2A-2D illustrate a method for assaying a secretome using a composition improving assay, in accordance with some embodiments.

FIGS. 3A-3E illustrate apparatuses for assaying a biological sample using a composition improving assay, in accordance with some embodiments.

FIG. 4 shows a plurality of partitions, in accordance with some embodiments.

FIG. 5 shows a plurality of partitions, in accordance with some embodiments.

FIG. 6 shows a transfer unit, in accordance with some embodiments.

FIG. 7 shows a transfer unit, in accordance with some embodiments.

FIG. 8 shows transfer units, in accordance with some embodiments.

FIG. 9 shows illustrate a method for assaying a secretome using a composition improving assay to determine a biological state, in accordance with some embodiments.

FIG. 10 shows an apparatus, in accordance with some embodiments.

FIG. 11 shows transfer units, in accordance with some embodiments.

FIG. 12 shows an apparatus and components thereof, in accordance with some embodiments.

FIG. 13 shows an apparatus and components thereof, in accordance with some embodiments.

FIG. 14 shows illustrates components of a cell culture medium, in accordance with some embodiments.

FIG. 15 shows a method for assaying a secretome using a composition improving assay, in accordance with some embodiments.

FIGS. 16A-16B show the mass of peptide and the number of protein groups identified from a secretome, respectively, in accordance with some embodiments.

FIGS. 17A-17B show the number of peptides and protein groups identified, respectively, from the mass spectrometry experiments, in accordance with some embodiments.

FIG. 18 shows the number of protein groups that were exclusively identified in the improved secretome composition or the direct digest, as well as the number of protein groups that were identified in both experiments, in accordance with some embodiments.

FIGS. 19A-19B show spectral count versus count rank of protein groups identified in an improved secretome composition and direct digest, respectively, in accordance with some embodiments.

FIG. 20 shows the number of protein groups that were exclusively identified or mutually identified by using some particles versus direct digest, in accordance with some embodiments.

FIGS. 21A-21C show violin plots of mass distribution, hydropathicity distribution, and isoelectric point distribution of the protein groups identified in direct digest and an improved secretome composition, respectively, in accordance with some embodiments.

FIGS. 22A-22F show the number of proteins identified in an improved secretome composition and direct digest for various protein keywords, in accordance with some embodiments. Protein keywords considered were: Phosphoproteins, Tumor Suppressors, Glycoproteins, Alzheimer's Disease, Cytokines, and Kinases.

FIGS. 23A-23C show different proteomic workflows, number of protein groups identified in the workflows, and the coefficient of variance of peptides in the workflows, in accordance with some embodiments.

FIGS. 24A-24B show a comparison of the number of protein groups and the number of peptides identified using a label-free DDA workflow versus a composition improving workflow using particles, in accordance with some embodiments.

FIG. 25 shows a computer system configured implement a method or a system of the present disclosure, in accordance with some embodiments.

FIGS. 26A-26I show non-limiting examples of surfaces, in accordance with some embodiments of the disclosure. FIG. 26A illustrates a non-limiting example of a surface functionalized at one or more regions for capturing biomolecules. FIG. 26B illustrates a non-limiting example of a surface comprising one or more wells or depressions for capturing biomolecules. For example, a functionalized surface may be disposed in a 96 well plate or a 384 well plate. FIG. 26C illustrates a non-limiting example of a surface disposed on one or more particles. In some embodiments, the one or more particles may be disposed in one or more wells or depressions. FIG. 26D illustrates a non-limiting example of a surface disposed on a plurality of particles packed in a channel or a porous material disposed in a channel. FIG. 26E illustrates a non-limiting example of a surface disposed on an inner surface of a channel. FIGS. 26F-26I illustrate non-limiting examples of surfaces in accordance with some embodiments of the disclosure. A surface may comprise 1, 2, 3, 4 or any number of distinct surface regions. In some cases, a surface may be disposed on a particle. In some cases, a particle may be a porous particle.

FIG. 27 schematically illustrates systems and methods for quality controlling biological samples, in accordance with some embodiments.

FIG. 28 shows an example plate layout for a single run of the Proteograph™ assay, in accordance with some embodiments.

FIG. 29 shows the number of protein groups identified by performing mass spectrometry on direct digest samples versus samples enriched using one or more surfaces, in accordance with some embodiments.

### DETAILED DESCRIPTION

Conditioned media of different cell cultures can be used for a variety of in vitro biological applications. In some cases, the in vitro biological applications may include characterizing biomolecules, which are secreted from the cell cultures into the conditioned media, under different conditions. In some cases, the characterization of the secreted biomolecules from the in vitro biological applications may help develop an insight into an observed biological function. However, in various in vitro systems, extraneous biomolecules may be provided to the cell culture. For example, a set of abundant proteins in fetal bovine serum (FBS) or proteins sourced from different organisms may be included in a conditioned media for cell culturing. The inclusion of extraneous biomolecules such as those comprised in FBS can add complexity to the conditioned media. The extraneous biomolecules can affect the dynamic range of protein concentration in the conditioned media, the extraneous biomolecules can suppress signals originating from secreted biomolecules (e.g., from the cell culture), or the added volume of the conditioned media may contribute to a dilution of molecules of interest with the conditioned media.

A collection of biomolecules secreted by a cell may be referred to as the secretome. The secretome may comprise a variety of biomolecules, including members of biomolecule groups that may be classified based on composition (e.g., proteins, lipids, carbohydrates, and their combinations, such as glycoproteins, lipoproteins, glycolipids), and members of biomolecule groups that may be classified based on function (e.g., antibodies, cytokines, chemokines, hormones, growth factors, etc.).

The added complexity, the wider dynamic range, or increased dilution in in vitro systems can present challenges, similar in some ways, to challenges in plasma proteomics. Plasma is estimated to comprise unique proteins across concentrations spanning at least 12 to 20 orders of magnitude (“magnitudes of dynamic range”). High-abundance biomolecules in plasma (e.g., albumin) may obscure the detection of low abundance and rare biomolecules. Similarly, the secretome can comprise biomolecules across a large dynamic range (e.g., greater than 10, 11, or 12 orders of magnitude), which can make it so that detection of biomolecules in the low abundance range is obscured by the presence of very high abundance biomolecules. An associated challenge is that the total available amount of biomolecules in the low abundance range may be too low for some techniques to detect at all.

In some aspects, the present disclosure provides systems and methods that can overcome some of these and other challenges by improving the composition of the secretome collected from a biosample. The composition of the secretome may be improved such that (1) high abundance biomolecules in supplemented media that obscure detection of relevant biomolecules in the secretome are depleted, (2) low abundance biomolecules in the secretome are enriched, and/or (3) the dynamic range of the secretome in a biosample is compressed by changing the relative abundance ratios of biomolecules in the secretome. The improved composition of the secretome then may be subsequently analyzed in various assays, including ones further provided in the present disclosure. The systems and methods of the present disclosure can enable discoveries and derivations of insights from the discoveries based on detection and/or identification of biomolecules in the secretome across a dynamic range that spans a larger number of magnitudes compared to some other assays.

The compositions of secretomes may be improved using various techniques. In some cases, a technique may comprise contacting a biological sample comprising the secretome with one or more engineered surfaces, wherein the one or more engineered surfaces are configured to adsorb or bind biomolecule such that (1) high abundance biomolecules in supplemented media that obscure detection of relevant biomolecules in the secretome are depleted, (2) low abundance biomolecules in the secretome are enriched, and/or (3) the dynamic range of the secretome in a biosample is compressed by changing the relative abundance ratios of biomolecules in the secretome. The adsorbed or bound biomolecules may be released from the one or more engineered surfaces, outputting the improved composition for use in subsequent assays for detection of the biomolecules.

In some aspects, the present disclosure describes systems and methods for applying a deep and scalable proteome profiling platform to analyze FBS or non-FBS based cell media directly on an automated Proteograph™ Product Suite. In some cases, the systems and methods may leverage at least one, two, three, four, or five nanoparticles (NPs), which can be engineered with different and/or distinct physicochemical properties to provide a broad coverage of complex proteomes at scale.

In some aspects, the present disclosure describes a method for identifying one or more biomolecules. In some cases, the method comprises providing a supplemented medium. In some cases, the method comprises generating a set of biomolecules by incubating cells in the supplemented medium under conditions sufficient for the cells to generate the set of biomolecules. In some cases, the method comprises contacting at least a portion of the supplemented medium with one or more surfaces to adsorb the set of biomolecules. In some cases, the method comprises removing the one or more surfaces and the set of biomolecules from the at least the portion of the supplemented medium to produce a separated sample. In some cases, the method comprises releasing, in the separated sample, the set of biomolecules from the one or more surfaces. In some cases, the method comprises detecting at least a subset of the set of biomolecules, thereby identifying one or more biomolecules.

In some aspects, the present disclosure describes an apparatus for assaying a biological sample. In some cases, the apparatus comprises a substrate comprising one or more surfaces. In some cases, the apparatus comprises one or more cell culture chambers comprising viable cells. In some cases, the apparatus comprises one or more loading units that are operably coupled to the substrate and the cell culture chamber. In some cases, the apparatus comprises a computer readable medium comprising machine-executable code that, upon execution by a processor, implements a method of the present disclosure. In some cases, the method may comprise providing one or more controlled environments to the cells in the one or more cell culture chamber for a predetermined duration of time. In some cases, the cells produce a biological sample in the cell culture chamber. In some cases, the method may comprise transferring at least a portion of the biological sample from the cell culture chamber to the substrate using the one or more loading units. In some cases, the transferring may thereby contact the at least the portion with the one or more surfaces to adsorb a biomolecule in the at least the portion of the biological sample onto the one or more surface. In some cases, the method may comprise assaying at least a portion of the biomolecule to detect the biomolecule in the biological sample.

In some aspects, the present disclosure provides a method for characterizing a biological preparation. In some embodiments, the method comprises contacting the biological preparation with one or more surfaces to adsorb a plurality of biomolecules in the biological preparation onto the one or more surfaces. In some embodiments, the plurality of biomolecules comprises a product biomolecule and an impurity. In some embodiments, the method comprises assaying the plurality of biomolecules to determine a deviation between a composition of the plurality of biomolecules and a reference composition. In some embodiments, the deviation is indicative of a purity or an activity of the biological preparation.

In some aspects, the present disclosure provides a method for detecting an impurity in a biological preparation. In some embodiments, the method comprises contacting the biological preparation with the one or more surfaces to adsorb a plurality of biomolecules in the biological preparation onto the one or more surfaces. In some embodiments, the method comprises assaying the plurality of biomolecules to detect a biomolecule fingerprint of the biological preparation. In some embodiments, the biomolecule fingerprint comprises a signature of the impurity in the plurality of biomolecules.

In some aspects, the present disclosure provides an apparatus for characterizing biological preparation. In some embodiments, the apparatus comprises a first chamber configured to hold the biological preparation. In some embodiments, the biological preparation comprises a plurality of biomolecules. In some embodiments, the plurality of biomolecules comprises a product biomolecule and an impurity. In some embodiments, the apparatus comprises a second chamber comprising one or more surfaces. In some embodiments, the apparatus comprises a loader operably coupled to the first chamber and the second chamber. In some embodiments, the loader is configured to transfer the biological preparation between the first chamber and the second chamber. In some embodiments, the apparatus comprises a computer readable medium for measuring a purity or an activity of the biological preparation comprising machine-executable code that, upon execution by a processor, implements a method for analyzing the biological preparation. In some embodiments, the method comprises contacting, using the loader, the biological preparation from the first chamber with the one or more surfaces in the second chamber to adsorb the plurality of biomolecules in the biological preparation onto the one or more surfaces.

In some aspects, the present disclosure provides an apparatus for detecting an impurity in a biological preparation. In some embodiments, the apparatus comprises a plurality of chambers in operable connection with one another. In some embodiments, the plurality of chambers comprises one or more surfaces. In some embodiments, the apparatus comprises one or more fluid transfer devices operably coupled to the plurality of chambers. In some embodiments, the apparatus comprises a computer readable medium for detecting the impurity in the biological preparation comprising machine-executable code that, upon execution by a processor, implements a method for analyzing the biological preparation. In some embodiments, the method comprises contacting, using the one or more fluid transfer devices, the biological preparation with the one or more surfaces to adsorb a plurality of biomolecules in the biological preparation onto the one or more surfaces.

In some aspects, the present disclosure provides a computer-implemented method for generating a quality metric for a biological preparation. In some embodiments, the computer-implemented method comprises receiving a plurality of mass spectrometry datasets for a plurality of polyamino acids in the biological preparation. In some embodiments, the plurality of polyamino acids comprises at least one product biomolecule and a plurality of impurities. In some embodiments, the computer-implemented method comprises generating a plurality of polyamino acid identifications and a plurality of polyamino acid abundances for the plurality of polyamino acids based on the plurality of mass spectrometry datasets. In some embodiments, the computer-implemented method comprises processing the plurality of polyamino acid identifications to output the quality metric for the biological preparation.

In some aspects, the present disclosure provides a computer-implemented system. In some embodiments, the computer-implemented system comprises a digital processing device comprising: at least one processor, an operating system configured to perform executable instructions, a memory, and a computer program including instructions executable by the digital processing device to analyze a plurality of mass spectrometry datasets. In some embodiments, the computer program comprises instructions executable to receive a plurality of mass spectrometry datasets obtained from a plurality of biological preparations. In some embodiments, the computer program comprises instructions executable to process the plurality of mass spectrometry datasets in real-time to generate a plurality of quality metrics for the plurality of biological preparations. In some embodiments, the computer program comprises instructions executable to provide process control instructions to a manufacturing process for producing the plurality of biological preparations.

### Systems and Methods for Cell Incubation

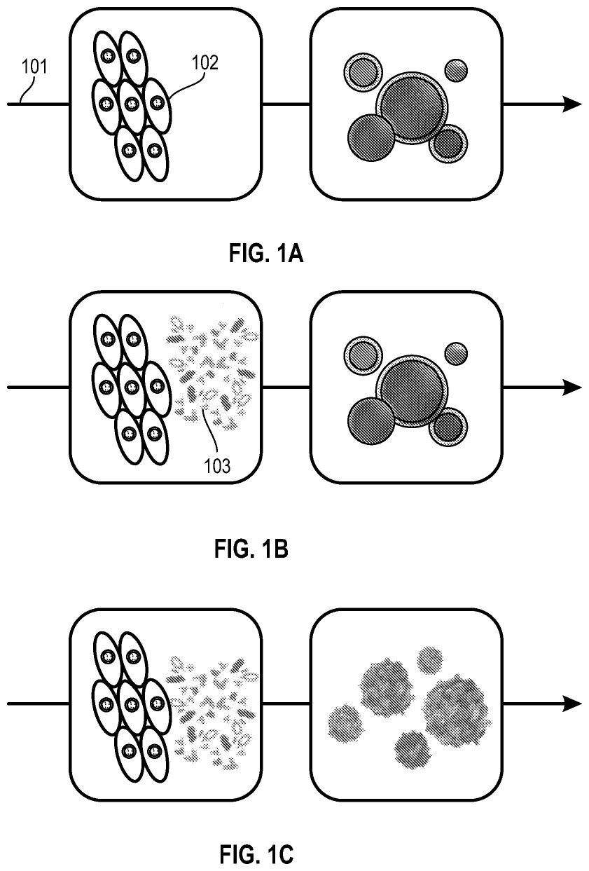

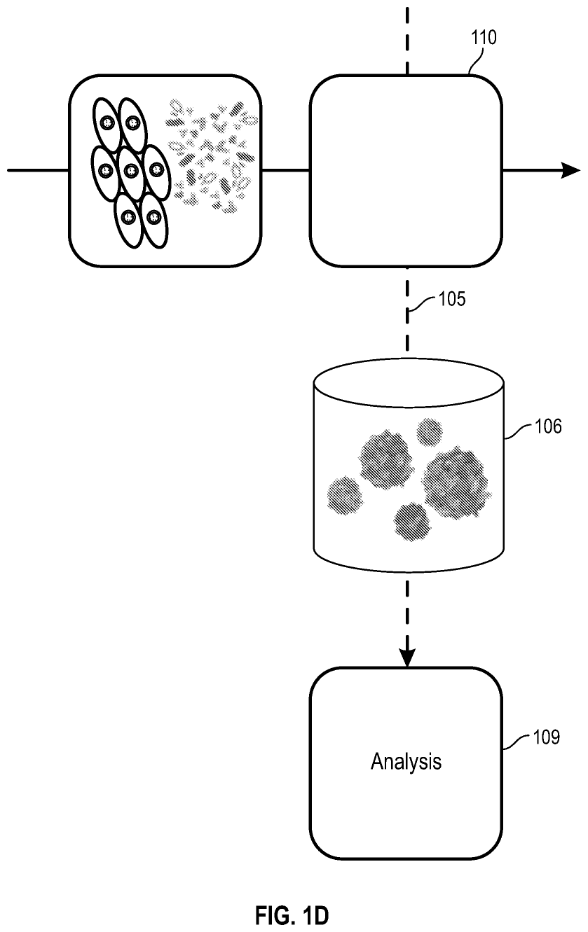

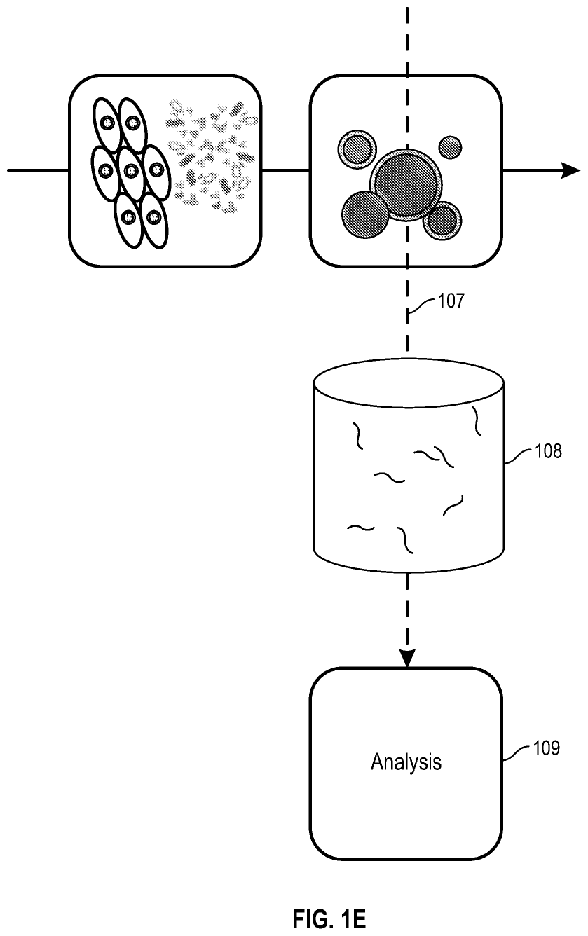

In some aspects, the present disclosure provides a method for incubating a cell. FIGS. 1A-1E illustrate a method for assaying a secretome using a composition improving assay, in accordance with some embodiments. In some cases, the method comprises providing a supplemented medium 101 to a cell 102. In some cases, the cell generates a set of biomolecules when the cell 102 is incubated in the supplemented medium 101. In some cases, the cell is incubated performed under conditions sufficient for the cell 102 to generate a set of biomolecules 103. In some cases, the set of biomolecules are contacted with one or more surfaces 104 to adsorb the set of biomolecules 103.

In some cases, the one or more surfaces 104 and the set of biomolecules 103 may be separated in operation 105 from a fluid composition 110 comprising the one or more surfaces 104 and the set of biomolecules 103 to produce a separated sample 106, as shown in FIG. 1D. In some cases, in the separated sample 106, the set of biomolecules 103 may be released from the one or more surfaces 104. In some cases, at least a subset of the set of biomolecules 103 may be detected using a detector (109), thereby identifying one or more biomolecules.

In some cases, the set of biomolecules 103 may be separated by operation 105 from a fluid composition 110 comprising the one or more surfaces 104 and the set of biomolecules 103, to produce a separated sample 108, as shown in FIG. 1E. In some cases, at least a subset of the set of biomolecules 103 may be detected using a detector (109), thereby identifying one or more biomolecules.

In some cases, the incubating is performed under conditions sufficient for the cell to generate the set of biomolecules. The conditions may comprise keeping constant or modulating various environmental factors and/or supplemented media compositions. The conditions may change the type or the amount of biomolecules released by the cell. In some cases, the conditions sufficient for the cell to exchange the set of biomolecules with the supplemented medium comprises a predetermined temperature, a predetermined pressure, a predetermined flow regime, a predetermined solvent environment, or a combination thereof. In some cases, the conditions sufficient for the cell to exchange the set of biomolecules with the supplemented medium comprises one or more of a presence or absence of an organic compound, one or more of a presence or absence of a therapeutic compound, a presence or absence of an inorganic compound, a presence or absence of an autocrine signaling molecule, a presence or absence of a paracrine signaling molecule, a presence or absence of an antigen, a presence or absence of one or more co-cultured cells, a presence or absence of radiation, a presence or absence of one or more toxins, a presence or absence of protein aggregates, a presence or absence of one or more proteins, a presence or absence of active viral particles, a presence or absence of inactivated viral particles, a presence or absence of applied heat or cooling, a presence or absence of applied mechanical stress, a presence or absence of electrical stimulation, a presence or absence of transposons, a presence or absence of exosomes, a presence or absence of liposomes, a presence or absence of coated nucleic acids, a presence or absence of shock, or any combination thereof. In some cases, the supplemented medium comprises a synthetic supplemented medium. In some cases, incubating the cell in the supplemented medium is performed while keeping conditions constant. In some cases, incubating the cell in the supplemented medium is performed at a first condition for a first amount time, then a second condition for a second amount of time. In some cases, incubating the cell in the supplemented medium is performed while continuously varying the condition. In some cases, incubating the cell in the supplemented medium is performed for less than about 5 seconds, 5 minutes, 5 hours, or 5 days. In some cases, incubating the cell in the supplemented medium is performed for at least about 5 seconds, 5 minutes, 5 hours, or 5 days. In some cases, incubating the cell in the supplemented medium is performed at a temperature of at least about 0, 5, 10, 15, 20, 25, 30, 35, 40, 45, 50, 55, 65, or 70 degrees Celsius. In some cases, incubating the cell in the supplemented medium is performed at a temperature of at most about 0, 5, 10, 15, 20, 25, 30, 35, 40, 45, 50, 55, 65, or 70 degrees Celsius. In some cases, incubating the cell in the supplemented medium is performed at an absolute pressure of at least about 0.01, 0.1, 1, 10, 100, 1000, 10000, or 100000 bars. In some cases, incubating the cell in the supplemented medium is performed at an absolute pressure of at most about 0.01, 0.1, 1, 10, 100, 1000, 10000, or 100000 bars. In some cases, incubating the cell in the supplemented medium is performed by flowing the supplemented medium over the cell at a Reynolds number of at least about 0.01, 0.1, 1, 10, 100, 500, 1000, 1500, 2000, 2500, 3000, 4000, 5000, 6000, 7000, 8000, or 9000. In some cases, incubating the cell in the supplemented medium is performed by flowing the supplemented medium over the cell at a Reynolds number of at most about 0.01, 0.1, 1, 10, 100, 500, 1000, 1500, 2000, 2500, 3000, 4000, 5000, 6000, 7000, 8000, or 9000. In some cases, incubating the cell in the supplemented medium is performed by flowing the supplemented medium in laminar flow. In some cases, incubating the cell in the supplemented medium is performed by flowing the supplemented medium in transient flow. In some cases, incubating the cell in the supplemented medium is performed by flowing the supplemented medium in turbulent flow. In some cases, the method comprises incubating the cell with a second supplemented medium. In some cases, the second supplemented medium comprises a second set of biomolecules. In some cases, the second supplemented medium is generated by incubating the cell in the supplemented medium for a different length of time.

In some cases, the supplemented medium comprises as a fraction or in whole a serum, a plasma, cerebral spinal fluid (CSF), synovial fluid (SF), urine, tears, crevicular fluid, semen, whole blood, milk, nipple aspirate, needle aspirate, ductal lavage, vaginal fluid, nasal fluid, ear fluid, gastric fluid, pancreatic fluid, trabecular fluid, lung lavage, prostatic fluid, sputum, fecal matter, bronchial lavage, fluid from swabbings, bronchial aspirants, sweat, saliva, or any combination thereof. In some cases, the supplemented medium comprises a supernatant of a cell culture, a secretome of co-cultures, tissue or cell lysate, or any combination thereof. In some cases, the supplemented medium comprises vectors. In some cases, the vectors comprise one or more engineered nucleic acids encoding a protein product.

In some cases, the method comprises generating a set of biomolecules by incubating a cell in the supplemented medium. The cell may generate various types of biomolecules. In some cases, the set of biomolecules comprises one or more biomarkers, molecular signatures, secreted proteins, absorbed proteins, exosomes, liposomes, lipids, hormones, glycoproteins, lipoproteins, glycolipids, peptides, cholesterols, carbohydrates, or any combination thereof. In some cases, the set of biomolecules comprises one or more recombinant or engineered biomolecules. In some cases, the exchange of the set of biomolecules comprises an active secretion of at least a portion of the set of biomolecules. In some cases, the exchange of the set of biomolecules comprises a passive release of at least a portion of the set of biomolecules. In some cases, the exchange of the set of biomolecules comprises use of an exosome or liposome by the cell. In some cases, the exchange of the set of biomolecules comprises apoptosis of the cell. In some cases, the apoptosis of the cell releases the set of biomolecules into solution. In some cases, released apoptotic biomolecules are exchanged with another cell. In some cases, the exchange of the set of biomolecules comprises necroptosis of the cell. In some cases, released necroptotic biomolecules are exchanged with another cell. In some cases, a biomolecule of the set of biomolecules is a complex. In some cases, a biomolecule of the set of biomolecules is a protein. In some cases, a biomolecule of the set of biomolecules is a polypeptide. In some cases, a biomolecule of the set of biomolecules is a nucleic acid. In some cases, a biomolecule in the set of biomolecules is a carbohydrate, a lipid, a protein, a glycolipid, a glycoprotein, a lipoprotein, or any macromolecular assembly thereof. In some cases, the one or more biomolecules secreted by the cell are conventional secretions, unconventional secretions, type 1 unconventional secretions, or type 2 unconventional secretions. In some cases, the set of biomolecules comprises a biomolecule assembly. In some cases, the biomolecule assembly comprises quaternary protein, a vesicle, or an exosome.

In some cases, the method comprises contacting at least a portion of the supplemented medium with one or more surfaces to adsorb the set of biomolecules. In some cases, the contacting is performed such that the portion of the supplemented medium contacts one or more surface regions of the one or more surfaces. In some cases, the one or more surfaces regions do not comprise a specific targeting moiety. In some cases, the surfaces may be comprised in one or more particles. In some cases, the surface regions may be comprised in one or more particles. In some cases, the surfaces may be comprised in a plurality of particles. In some cases, the surface regions may be comprised in a plurality of particles.

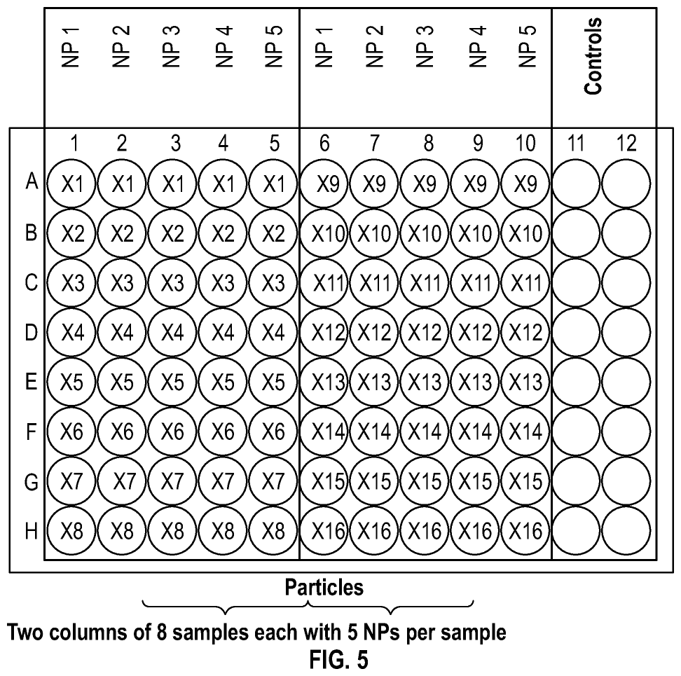

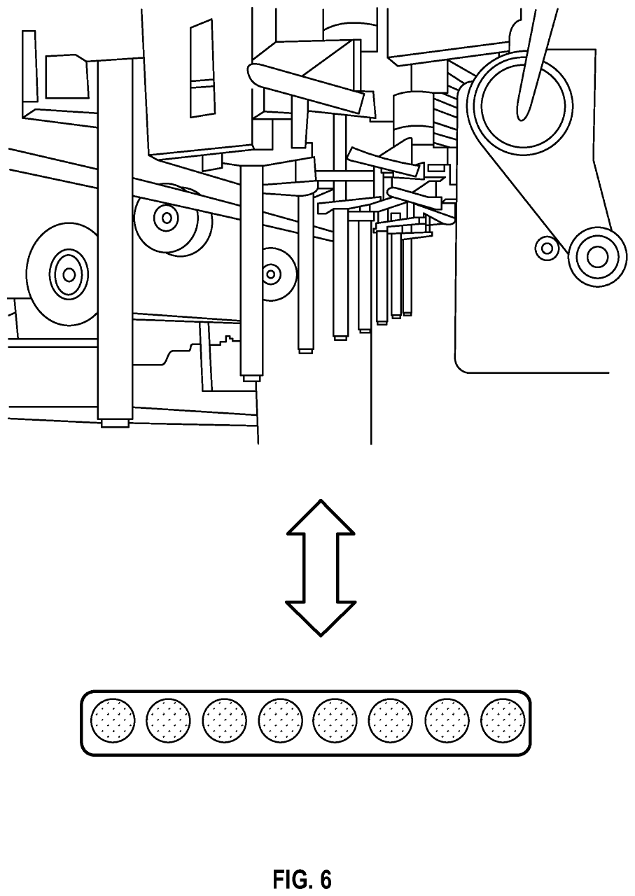

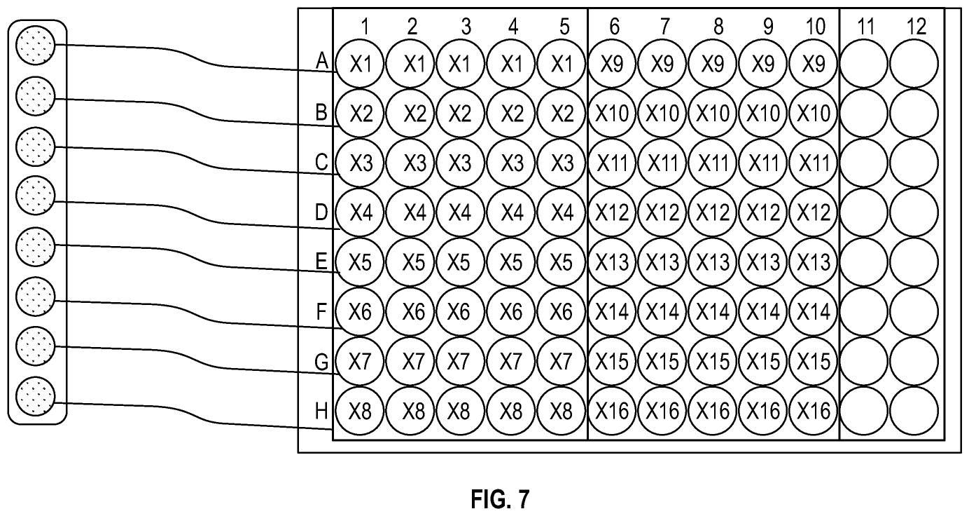

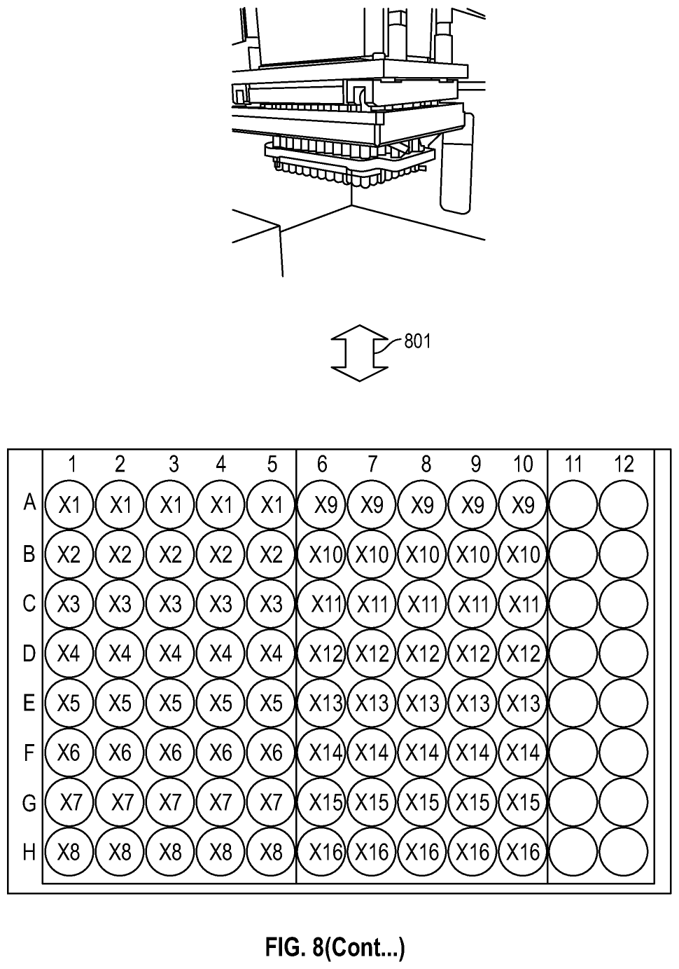

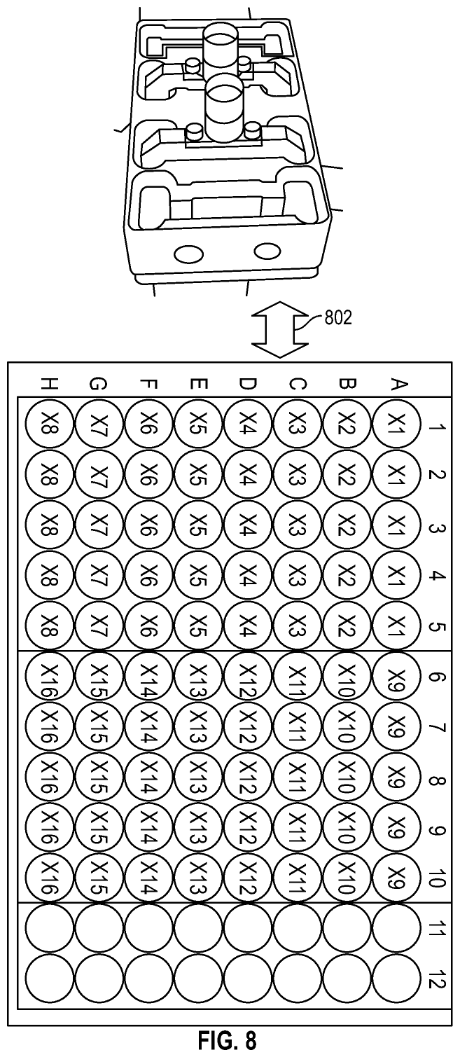

In some cases, the contacting may comprise aliquoting the at least the portion of the supplemented medium, and then providing the at least the portion of the supplemented medium in a chamber comprising the one or more surfaces. In some cases, the aliquoting may be performed using one or more transfer units. In some cases, the one or more transfer units may comprise a pipette head, as shown in FIG. 6 or FIG. 8. In some cases, the one or more transfer units may comprise fluidic connections, as shown in FIG. 7.

In some cases, the contacting may comprise providing the one or more surfaces to a chamber comprising the supplemented medium. For instance, the one or more surfaces may be provided on one or more particles which are added to a partition comprising the supplemented medium and/or a cell. In some cases, the one or more particles may be provided in a fluid composition. In some cases, the fluid composition may be added to the partition. In some cases, the fluid composition may be transferred using one or more transfer units. In some cases, the one or more transfer units may comprise a pipette head, as shown in FIG. 6 or FIG. 8. In some cases, the one or more transfer units may comprise fluidic connections, as shown in FIG. 7. In some cases, the set of biomolecules are improved in composition for detecting biomolecules. In some cases, the set of biomolecules comprises a reduced dynamic range when adsorbed on the one or more surfaces compared to an original dynamic range of the set of biomolecules in the supplemented medium. In some cases, subsequent to the contacting, the set of biomolecules are depleted in high abundance biomolecules. In some cases, subsequent to the contacting, the set of biomolecules are depleted in easily detectable biomolecules. In some cases, subsequent to the contacting, the set of biomolecules are enriched in low abundance biomolecules. In some cases, subsequent to the contacting, the set of biomolecules are enriched in difficult to detect biomolecules.

In some cases, the contacting comprises contacting the biological sample with a second surface to adsorb a second plurality of biomolecules onto the second surface. In some cases, subsequent to the second contacting, the set of biomolecules are improved in composition for detecting biomolecules. In some cases, the set of biomolecules comprises a reduced dynamic range when adsorbed on the one or more surfaces compared to an original dynamic range of the set of biomolecules in the supplemented medium. In some cases, subsequent to the contacting, the set of biomolecules are depleted in high abundance biomolecules. In some cases, subsequent to the contacting, the set of biomolecules are depleted in easily detectable biomolecules. In some cases, subsequent to the contacting, the set of biomolecules are enriched in low abundance biomolecules. In some cases, subsequent to the contacting, the set of biomolecules are enriched in difficult to detect biomolecules.

In some cases, the method comprises releasing, in the separated sample, the set of biomolecules from the one or more surfaces. In some cases, the releasing comprises use of an enzyme. In some cases, the releasing comprises use of a protease. In some cases, the releasing comprises use of a trypsin. In some cases, the releasing comprises use of a lysin. In some cases, the releasing comprises use of a buffer. In some cases, the releasing comprises use of a solvent. In some case, the releasing comprises fragmenting the biomolecules.

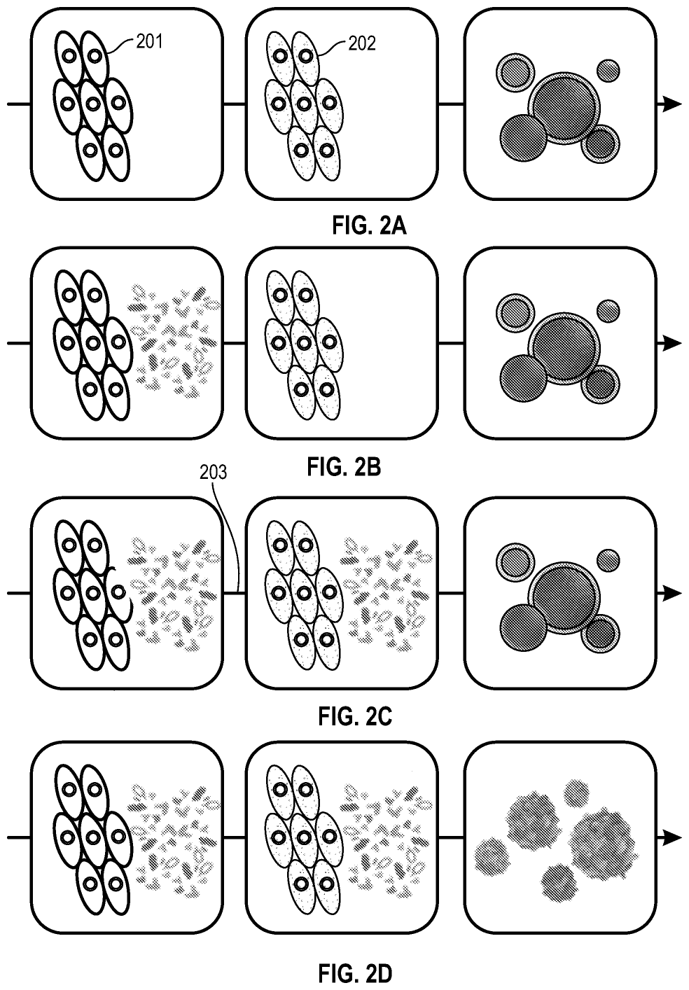

FIGS. 2A-2D illustrate a method for assaying a secretome using a composition improving assay, in accordance with some embodiments. In some cases, the plurality of cells comprises at least a first cell 201 of a first type and a second cell 202 of a second type, such that the first cell exchanges 203 one or more biomolecules of the set of biomolecules with the second cell. In some cases, the first cell and the second cell are co-cultured. In some cases, the first cell is comprised in a feeder culture for the second cell. In some cases, the plurality of cells is disposed in a plurality of separate volumes. In some cases, each volume in the plurality of separate volumes comprises a different supplemented medium or incubating conditions.

In some cases, the method further comprises determining if the one or more biomolecules were generated by the cell and not originally present in the supplemented medium. In some cases, at least a portion of biomolecules in the supplemented medium are depleted. In some cases, the supplemented medium comprises fetal bovine serum. In some cases, fetal bovine serum is depleted.

In some cases, the method comprises monitoring cell activity. In some cases, the method comprises incubating a cell such that the cell generates a biological sample comprising a plurality of biomolecules. In some cases, the method comprises contacting the biological sample with a surface to adsorb the plurality of biomolecules onto the surface. In some cases, the method comprises releasing at least a portion of the plurality of biomolecules on the surface. In some cases, the method comprises detecting the at least the portion of the plurality of biomolecules, thereby identifying the plurality of biomolecules. In some cases, the method comprises analyzing the activity of the cell.

In some cases, the method comprises incubating a cell such that the cell generates a second biological sample comprising a second plurality of biomolecules after a predetermined amount of time. In some cases, the method comprises contacting the second biological sample with a second surface to adsorb the second plurality of biomolecules onto the surface after the predetermined amount of time. In some cases, the method comprises releasing at least a second portion of the second plurality of biomolecules on the second surface after a predetermined amount of time. In some cases, the method comprises detecting the at least the second portion of the second plurality of biomolecules, thereby identifying the second plurality of biomolecules after a predetermined amount of time. In some cases, the cell generates the biological sample by any one of producing, releasing, adsorbing, digesting, or modifying a biomolecule of the plurality of biomolecules. In some cases, the cell adds to a supplemented medium by any one of producing, releasing, adsorbing, digesting, or modifying a biomolecule of the plurality of biomolecules.

In some cases, the method comprises generating a plurality of biological samples at a plurality of different times. In some cases, the plurality of different times may be used to monitor changes in cell activity, biological sample composition, or both. For example, a cell may be incubated under the same or changing conditions for some time period (e.g., minutes, hours, days, weeks, months, or years), and the cell's excreted biomolecules may be sampled periodically (e.g., in seconds, minutes, hours, days, weeks, or months). The periodically sampled biomolecules may be assayed to generate a time series that can elucidate dynamics of the cell's activity through time. Changes in the cell's excreted biomolecules may indicate changes in the activity of the cell.

In some cases, the method comprising changing an incubation condition for the cell based at least in part on the activity of the cell. In some cases, the incubation condition changed may comprise: temperature, pressure, flow rate, supplemented medium composition, or any combination thereof.

In some cases, the method comprises identifying a low abundance biomolecule in a biological sample. In some cases, the method comprises incubating a cell in a predetermined environment. In some cases, the cell produces a biological sample. In some cases, the method comprises adding an extraneous biomolecule to the biological sample. In some cases, the extraneous biomolecule reduces a detectability of the plurality of low abundance biomolecules in the biological sample. In some cases, the extraneous biomolecule may be a biomolecule that is not endogenously expressed or synthesized by the cell. In some cases, the method comprises contacting the biological sample with a surface to adsorb a plurality of low abundance biomolecules in the biological sample onto the surface. In some cases, the method comprises releasing at least a portion of the plurality of low abundance biomolecules on the surface. In some cases, the method comprises detecting the at least the portion of the plurality of low abundance biomolecules. In some cases, the method comprises identifying the plurality of low abundance biomolecules.

In some cases, the detecting a low abundance biomolecule is at least about 7 orders of magnitude more probable when the biological sample is contacted with a surface. In some cases, the detecting a low abundance biomolecule may be at least about 5, 6, 7, 8, 9, 10, 11, 12, 13, 14, or 15 orders of magnitude more probable when the biological sample is contacted with a surface. In some cases, the detecting a low abundance biomolecule may be at most about 5, 6, 7, 8, 9, 10, 11, 12, 13, 14, or 15 orders of magnitude more probable when the biological sample is contacted with a surface. In some cases, a low abundance biomolecule in the set of biomolecules may be at least about 7 orders of magnitude lower in abundance or concentration than a high abundance biomolecule in the set of biomolecules. In some cases, a low abundance biomolecule in the set of biomolecules may be at least about 5, 6, 7, 8, 9, 10, 11, 12, 13,14, or 15 orders of magnitude lower in abundance or concentration than a high abundance biomolecule in the set of biomolecules. In some cases, a low abundance biomolecule in the set of biomolecules may be at most about 5, 6, 7, 8, 9, 10, 11, 12, 13,14, or 15 orders of magnitude lower in abundance or concentration than a high abundance biomolecule in the set of biomolecules.

In some cases, the set of biomolecules, when identified, may lead to insights regarding the biological processes occurring in the cell. In some cases, the method comprises determining one or more protein to protein interactions, biomarkers, molecular signatures, biomolecules absorbed by the cell, biomolecules secreted by the cell, biomolecules dissociating from cell surfaces, biomolecules being cleaved off or shed from the surface, macromolecular complexes budded, biomolecules passively released by the cell, conventionally and unconventionally released proteins, apoptotic release of biomolecules, necrotic released biomolecules, post-translation modifications, cell to cell interactions, cell to cell communications, or any combination thereof based at least in part on the identification. In some cases, molecular signatures may comprise a pattern of proteins and/or proteoforms indicative of a biological state.

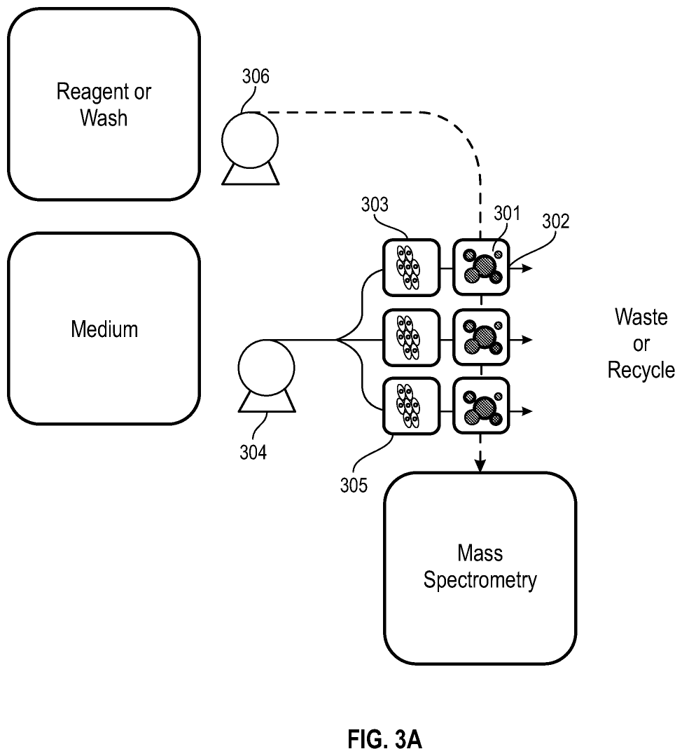

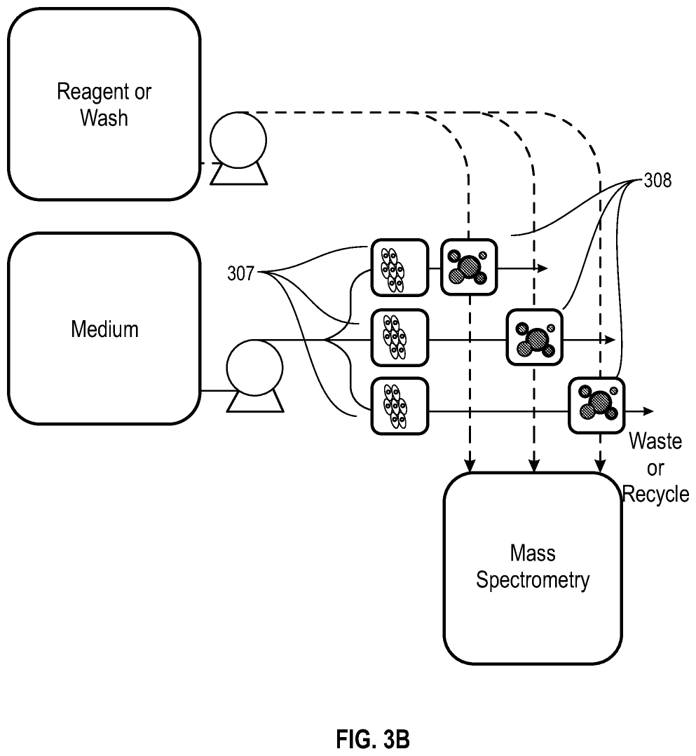

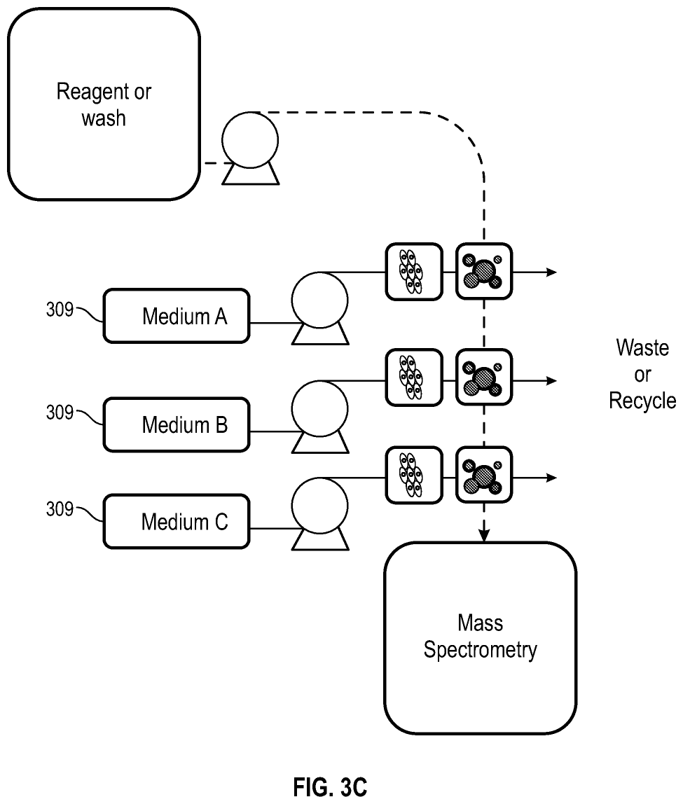

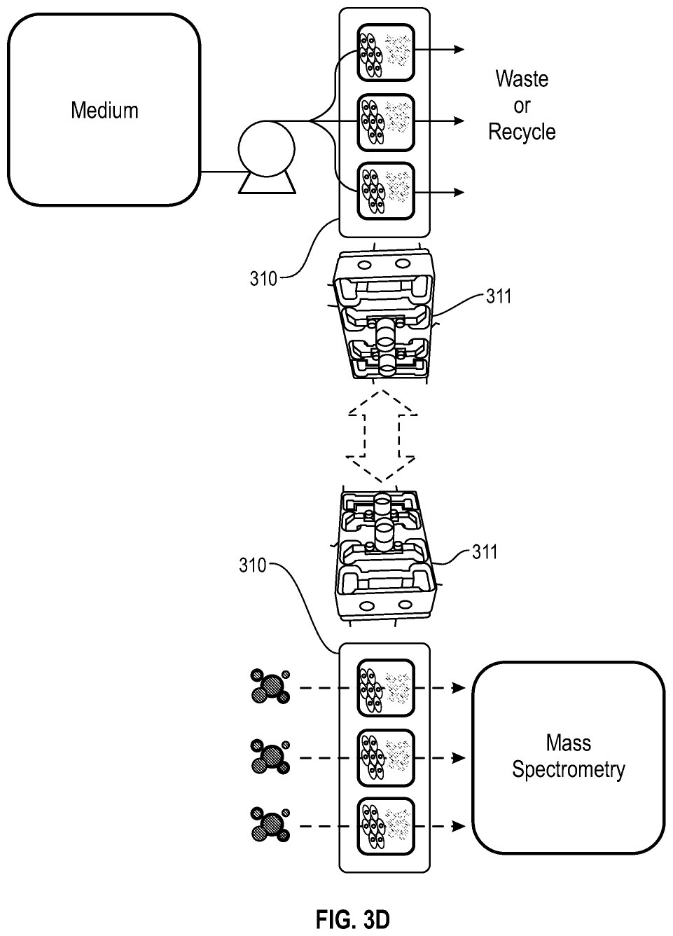

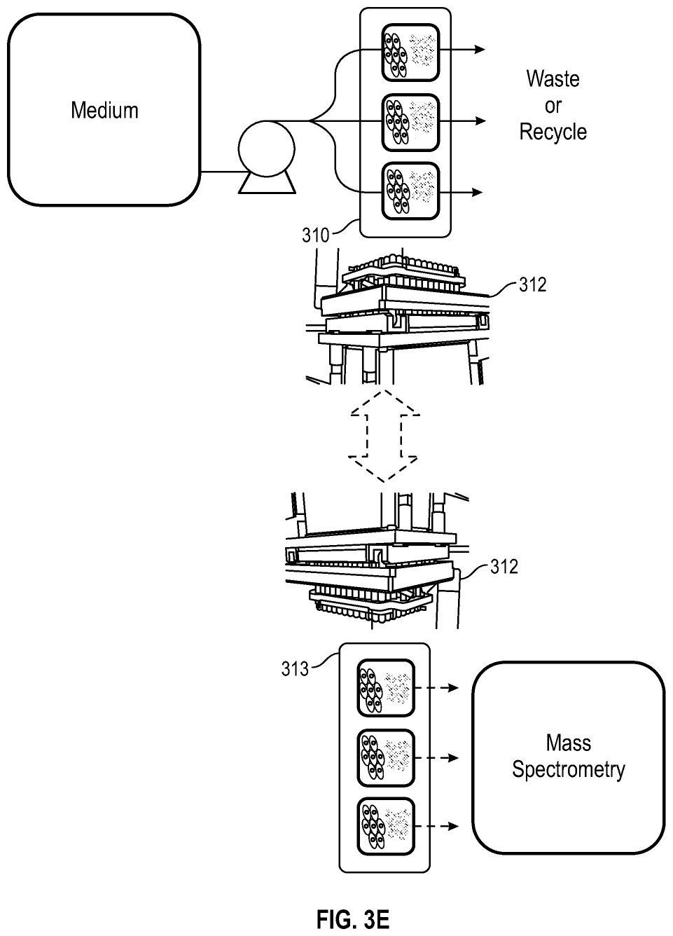

In some aspects, the present disclosure provides an apparatus for assaying a biological sample. FIG. 3A illustrates an apparatus for assaying a biological sample using a composition improving assay, in accordance with some embodiments. In some cases, the apparatus comprises a substrate 301 comprising a surface. In some cases, the substrate 301 may be disposed in a substrate chamber 302. In some cases, the apparatus comprises a cell culture chamber 303 comprising a cell 305. In some cases, the apparatus comprises a loading unit 304. In some cases, the loading unit is operably coupled to the substrate chamber 302, the cell culture chamber 303, or both. In some cases, the loading unit 304 may comprise a pump, a pipetting machine, a sample holder, or any other device configured to transfer at least a portion of a biological sample from one location to another. In some cases, the cell culture chamber 303 and the substrate chamber 302 may be comprised in a sample volume. In some cases, the cell culture chamber 303 and the substrate chamber 302 may comprise distinct volumes. In some cases, the distinct volumes may be fluidically connected. In some cases, the apparatus comprises a second loading unit 306. In some cases, the second loading unit is operably coupled to the substrate chamber 301, the cell culture chamber 302, or both. In some cases, the second loading unit 306 is configured transfer at least a portion of the biological sample from one location from another. In some cases, the second loading unit 306 is configured to transfer the at least the portion of the biological sample to a location than the location that the first loading unit 304 is configured to transfer the at least the portion of the biological sample.

FIG. 3B illustrates an apparatus for assaying a biological sample using a composition improving assay, in accordance with some embodiments. In some cases, the apparatus may comprise a plurality of different cell lines 307. In some cases, the plurality of different cell lines 307 may be incubated under the same conditions. For example, the apparatus may comprise a plurality of partitions (e.g., a 96 well-plate) comprising a plurality of different cell lines. The plurality of different cell lines 307 may be incubated under the same conditions. In some cases, a plurality of biological samples may be obtained from the plurality of different cell lines 307. In some cases, the plurality of biological samples may be assayed to identify biomolecules generated by each cell line in the plurality of cell lines 307. In some cases, the plurality of biological samples may be assayed with a plurality of surface types 308. In some cases, the plurality of surface types may comprise a plurality of different surface chemistries.

FIG. 3C illustrates an apparatus for assaying a biological sample using a composition improving assay, in accordance with some embodiments. In some cases, the apparatus may comprise a plurality of different supplemented media 309. In some cases, the plurality of different supplemented media 309 may be used to incubate one or more cells in a plurality of partitions. In some cases, the one or more cells may comprise one or more cell lines. In some cases, different biomolecules may be excreted by cells exposed to different supplement media. In some cases, a plurality of biological samples may be obtained from the plurality of partitions, each comprising one or more cells incubated with one of the plurality of different supplemented media 309. In some cases, the plurality of partitions may contain the same cell line (e.g., HeLa cells) but different supplemented media 309. For example, the supplemented media 309 may contain different organic compounds, such as drugs. In some cases, the plurality of biological samples may be assayed to identify biomolecules generated by the one or more cells under the influence of the various supplemented media 309. In some cases, the plurality of biological samples may be assayed with one or more surface types. In some cases, the one or more surface types may comprise the same or a plurality of different surface chemistries.

FIG. 3D illustrates an apparatus for assaying a biological sample using a composition improving assay, in accordance with some embodiments. In some cases, the apparatus may comprise a plurality of partitions 310 comprising a plurality of cells. In some cases, the plurality of cells may be incubated with a supplemented medium. In some cases, the plurality of partitions may be transferred using a transfer unit 311 from a first location to a second location. In some cases, one or more particles may be added to the plurality of partitions to assay one or more biomolecules in the plurality of partitions.

FIG. 3E illustrates an apparatus for assaying a biological sample using a composition improving assay, in accordance with some embodiments. In some cases, the apparatus may comprise a first plurality of partitions 310 comprising a plurality of cells. In some cases, the plurality of cells may be incubated with a supplemented medium. In some cases, a plurality of samples from the first plurality of partitions may be transferred to a second plurality of partition 313 using a transfer unit 312. In some cases, the second plurality of partitions 313 may comprise one or more surfaces for assaying one or more biomolecules in the plurality of samples.

In some cases, a loading unit is configured to input a reagent, a wash, or a supplemented medium into a chamber. In some cases, the first loading unit, the second loading unit, or both, are configured to input a reagent, a wash, or a supplemented medium into the cell culture chamber, the substrate chamber, or both.

In some cases, the apparatus comprises a computer readable medium comprising machine-executable code that, upon execution by a processor, implements any method disclosed herein. In some cases, the method comprises providing a controlled environment to the cell in the cell culture chamber for a predetermined duration of time. In some cases, the cell produces a biological sample in the cell culture chamber. In some cases, the method comprises transferring at least a portion of the biological sample from the cell culture chamber to the substrate using the loading unit. In some cases, the method comprises contacting the at least the portion with the surface to adsorb a biomolecule in the at least the portion of the biological sample onto the surface. In some cases, the method comprises transferring at least the portion of the biological sample to an analytical instrument. In some cases, the analytical instrument comprises a mass spectrometer. In some cases, the analytical instrument comprises a sequencer. In some cases, the analytical instrument comprises a chromatography column. In some cases, the method comprises assaying at least a portion of the biomolecule to detect the biomolecule in the biological sample. In some cases, the method comprises assaying at least a portion of the biomolecule to detect the biomolecule in the biological sample using the analytical instrument. In some cases, the apparatus comprising a plurality of cell culture chambers. In some cases, a first cell culture chamber 303 in the plurality of cell culture chambers is provided with a first controlled environment. In some cases, a second cell culture chamber 305 in the plurality of cell culture chambers is provided with a second controlled environment.

In some aspects, the present disclosure provides a method for identifying one or more biomolecules. In some cases, the method comprises incubating a cell under conditions sufficient for the cell to generate an exosome. In some cases, the exosome comprises a plurality of biomolecules. In some cases, the method comprises contacting the exosome with one or more surfaces to capture at least a portion of the exosome. In some cases, the method comprises removing the one or more surfaces and the at least the portion of the exosome from the cell to produce a separated sample. In some cases, the method comprises releasing, in the separated sample, the at least the portion of the exosome from the one or more surfaces. In some cases, the method comprises detecting at least a subset of the plurality of biomolecules in the at least the portion of the exosome, thereby identifying one or more biomolecules.

In some aspects, the present disclosure provides a method for monitoring cell activity. In some cases, the method comprises incubating a cell such that the cell generates a biological sample comprising an exosome. In some cases, the exosome comprises a plurality of biomolecules. In some cases, the method comprises contacting the biological sample with a surface to capture at least a portion of the exosome onto the surface. In some cases, the method comprises releasing the at least the portion of the exosome from the surface. In some cases, the method comprises detecting the at least the portion of the plurality of biomolecules in the at least the portion of the exosome. In some cases, the method comprises identifying the plurality of biomolecules. In some cases, the method comprises repeating any one or combination of steps after a predetermined amount of time. In some cases, the method comprises monitoring an activity of the cell. In some cases, the exosome may be enriched from a biological sample before contacting with one or more surfaces. In some cases, the exosome may be enriched using size-exclusion chromatography. In some cases, the exosome may be enriched using differential ultracentrifugation. In some cases, the exosome may be enriched using ultrafiltration, precipitation, or immunoaffinity methods.

In some aspects, the present disclosure provides a method for identifying a low abundance biomolecule in a biological sample. In some cases, the method comprises incubating a cell in a predetermined environment. In some cases, the cell produces a biological sample comprising an exosome. In some cases, the exosome comprises a plurality of low-abundance biomolecules. In some cases, the method comprises contacting the biological sample with a surface to capture at least a portion of the exosome in the biological sample onto the surface. In some cases, the method comprises releasing the at least the portion of the exosome from the surface. In some cases, the method comprises detecting at least the portion of the plurality of low abundance biomolecules in the at least the portion of the exosome. In some cases, the method comprises identifying the plurality of low abundance biomolecules. In some cases, the plurality of low-abundance biomolecules comprises less than about 1, 1×10⁻¹, 1×10⁻², 1×10⁻³, 1×10⁻⁴, 1×10⁻⁵, 1×10⁻⁶, 1×10⁻⁷, or 1×10⁻⁸ percent by mass of biomolecules in the exosome. In some cases, the plurality of low-abundance biomolecules comprises more than about 1, 1×10⁻¹, 1×10⁻², 1×10⁻³, 1×10⁻⁴, 1×10⁻⁵, 1×10⁻⁶, 1×10⁻⁷, or 1×10⁻⁸ percent by mass of biomolecules in the exosome.

In some aspects, the present disclosure provides an apparatus for assaying a biological sample. In some cases, the apparatus comprises a substrate comprising a surface. In some cases, the apparatus comprises a cell culture chamber comprising a cell. In some cases, the apparatus comprises a loading unit that is operably coupled to the substrate and the cell culture chamber. In some cases, the apparatus comprises a computer readable medium comprising machine-executable code that, upon execution by a processor. In some cases, the computer readable medium implements a method comprising providing a controlled environment to the cell in the cell culture chamber for a predetermined duration of time. In some cases, the cell produces a biological sample comprising an exosome in the cell culture chamber. In some cases, the exosome comprises one or more biomolecules. In some cases, the computer readable medium implements a method comprising transferring at least a portion of the biological sample from the cell culture chamber to the substrate using the loading unit. In some cases, the computer readable medium implements a method comprising contacting the at least the portion with the surface to adsorb the exosome in the at least the portion of the biological sample onto the surface. In some cases, the computer readable medium implements a method comprising assaying the exosome to detect the one or more biomolecules in the biological sample.

In some aspects, the present disclosure provides a method of identifying biomolecules. In some cases, the method comprises processing one or more exosomes to release a plurality of biomolecules in the one or more exosomes to an environment external to the one or more exosomes. In some cases, a subset of biomolecules in the plurality of biomolecules comprises a first distribution of relative abundances in the one or more exosomes. In some cases, the method comprises performing a composition improving assay on the plurality of biomolecules to increase the first distribution to a second distribution of relative abundances for the subset of biomolecules. In some cases, the method comprises assaying the plurality of biomolecules to identify the subset of biomolecules.

In some aspects, the present disclosure provides a method of identifying biomolecules. In some cases, the method comprises contacting a biological sample comprising one or more exosomes with a plurality of particles. In some cases, the method comprises non-specifically binding a subset of the one or more exosomes on the plurality of particles. In some cases, the plurality of particles comprises distinct physicochemical properties. In some cases, the one or more exosomes comprise a plurality of biomolecules. In some cases, the method comprises processing the one or more exosomes to release the plurality of biomolecules to an environment external to the one or more exosomes. In some cases, the method comprises assaying the plurality of biomolecules to identify at least a subset of biomolecules in the plurality of biomolecules.

### Supplemented Media

Supplemented media may comprise various chemical constituents that may influence a cell's secretome composition. In some cases, a supplemented medium may comprise one or more nutrients, one or more drugs, one or more therapeutic compounds, one or more biomolecules, one or more pathogens, or any combination thereof. In some cases, a supplemented medium may provide a supply of nutrients for one or more cells to grow and/or proliferate. In some cases, a supplemented medium may comprise one or more substances that reduce visibility or detectability of at least a portion of biomolecules excreted by a cell in a downstream assay. For example, some nutrients in a supplemented medium may be proteins that are provided in high abundance (compared to biomolecules of interest that are excreted or potentially excreted by one or more cells). The high abundance proteins may obscure detection of low abundance biomolecules that are produced by the one or more cells.

In some embodiments, a nutrient may comprise any substance used by a cell in metabolism. In some embodiments, a nutrient is or comprises a carbohydrate, a lipid, a fiber, a mineral, a protein, a vitamin, microbes, water, a sugar, a fat, an ion, an amino acid, a peptide, an alcohol, a ketone, a carboxylic acid, an amine, an amide, an ether, an ester, a base, an acid, a small molecule supplement, a symbiotic gut bacterium, a hormone, salt of a nutrient, a metabolite of a nutrient, a synthetically altered form of a nutrient, a micronutrient, an isotope of a nutrient, or any combination thereof.

In some embodiments, a drug may be configured to perturb the cell. Any drug or candidate drug may be contemplated with the systems and methods of the present disclosure. In some embodiments, a drug may comprise a chemotherapeutic agent, hormone targeting agent, an agent configured to target a metabolic reaction, or an agent that is configured to influence a regulator of a metabolic reaction. In some embodiments, a drug can comprise an analgesic, an anesthetic, an antibacterial, an antibiotic, an anticonvulsant, an antidementia agent, an antidepressant, an antidote, an antitoxin, an antiemetic, an antifungal, an anti-inflammatory agent, a corticosteroid, a nonsteroidal anti-inflammatory agent, an antimigraine agent, an antimyasthenic agent, an antimycobacterial, an antineoplastic, an antiparasitic, an antiparkinson agent, an antipsychotic, an antiviral, an antiretrovial, an anti hepatitis C agent, an anxiolytic, an anti-anxiety agent, a bipolar agent, a blood glucose regulator, insulin, a blood product, an anticoagulant, a cardiovascular agent, a beta-blocker, an ACE inhibitor, a central nervous system agent, an amphetamine, a dental agent, an oral agent, a dermatological agent, an enzyme replacement agent, a gastrointestinal agent, an H2 blocker, a proton pump inhibitor, a genitourinary agent, a genitourinary agent for genital tract, a genitourinary agent for urinary tract agent, a hormonal agent, an adrenal hormonal agent, a pituitary hormonal agent, a prostaglandin hormonal agent, a sex hormone, estrogen, testosterone, anabolic steroids, a thyroid agent, a hormonal suppressant, an adrenal hormone suppressant, a parathyroid hormone suppressant, a pituitary hormone suppressant, a sex hormone suppressant, a thyroid hormone suppressant, an immunological agent, a vaccine, an anti-rheumatic drug, an inflammatory bowel disease agent, a metabolic bone disease agent, an ophthalmic agent, an otic agent, a respiratory tract agent, an antihistamine, a bronchodilator, a sedative, a hypnotic, a skeletal muscle relaxant, a therapeutic nutrient, a mineral, an electrolyte, a chemotherapy drug, a radiation therapy drug, or any combination thereof.

The supplemented medium may comprise water and/or various cosolvents. In some cases, the supplemented medium may comprise water, methanol, ethanol, propanol, butanol, acetone, polyethylene glycol (PEG), dimethyl sulfoxide (DMSO), beta-cyclodextrin, buffers, or any combination thereof.

In some cases, the supplemented medium may comprise a natural medium. In some cases, the supplemented medium may comprise an artificial medium. In some cases, an artificial medium may comprise organic nutrients, inorganic nutrients, vitamins, salts, O₂, CO₂, serum proteins, carbohydrates, cofactors, or any combination thereof. In some cases, the artificial medium may be configured to improve survival of a cell in a short term, improve survival of a cell in a long term, promote growth or proliferation of a cell, or any combination thereof.

In some cases, the supplemented medium may comprise a serum containing medium. In some cases, the serum containing medium may comprise fetal bovine serum. In some cases, the serum containing medium may comprise carriers or chelators for solubilizing labile or water-insoluble nutrients, hormones and growth factors, protease inhibitors, or any combination thereof. In some cases, the serum containing medium may bind and/or neutralizes toxins.

In some cases, the supplemented medium may comprise a serum-free medium. In some cases, the serum-free medium may comprise Knockout Serum Replacement, Knockout DMEM, and mTESR medium, purified growth factors, lipoproteins, or any combination thereof.

In some cases, the supplemented medium may comprise a chemically defined medium. In some cases, the chemically defined medium may comprise inorganic constituents, organic constituents, protein additives (e.g., growth factors), or any combination thereof.

In some cases, the supplemented medium may comprise a natural buffering system. In some cases, the natural buffering system may comprise CO₂ which may balance with CO3/HCO3 content in the supplemented medium.

In some cases, the supplemented medium may comprise an inorganic salt, an amino acid, carbohydrates, proteins, peptides, fatty acids, lipids, vitamins, trace elements, antibiotics, albumins, growth factors, growth inhibitors hormones, transferrin, fibronectin, protease inhibitors, or any combination thereof.

In some cases, the supplemented medium may comprise HEPES, phenol red, Dulbecco's Modified Eagle's Medium (DMEM), RPMI-1640, Eagle's Minimum Essential Medium (EMEM), any one of Ham's nutrient mixtures, Ham's F-10, Ham's F-12, Coon's modification of Ham's F-12, DMEM/F12, Iscove's Modified Dulbecco's Medium (IMDM), neurobasal medium, McCoy's 5A medium, Dynamis medium, Essential 8 (E8) media, StemFlex culture media, Airway Epithelial Cell basal medium, alpha-modified minimum essential medium (α-MEM), StemMacs iPS-Brew media, TeSR-E8, mTeSR1, mTeSR Plus, GMEM (Glasgow Minimum Essential Medium), Opti-MEM I, SmGM-2, fibroblast growth media/FGM, StemPro-34 serum free growth medium, mTESR1 medium, ECGM-2 media, EGM2-Bullet kit medium, Williams' Medium E, medium M254, CnT07 media, TNM-FH media, mammary epithelial cell growth basal medium (MEBM), complete skeletal muscle media, NeuroCult NS-A basal medium, Ham's F-10 [50], Fibroblast Growth Kit-Low Serum, or any combination thereof.

In some cases, the supplemented medium comprises a pH of at least about 1, 2, 3, 4, 5, 6, 7, 8, 9, 10, 11, 12, 13, or 14. In some cases, the supplemented medium comprises a pH of at most about 1, 2, 3, 4, 5, 6, 7, 8, 9, 10, 11, 12, 13, or 14. In some cases, the supplemented medium comprises an oxygen concentration of at least about 0.001, 0.01, 0.1, 1, 10, 100, 1000, 10000 mg/L. In some cases, the supplemented medium comprises an oxygen concentration of at most about 0.001, 0.01, 0.1, 1, 10, 100, 1000, 10000 mg/L. In some cases, the supplemented medium comprises a carbon dioxide concentration of at least about 0.001, 0.01, 0.1, 1, 10, 100, 1000, 10000 mg/L. In some cases, the supplemented medium comprises a carbon dioxide concentration of at most about 0.001, 0.01, 0.1, 1, 10, 100, 1000, 10000 mg/L. In some cases, the supplemented medium comprises an ammonia concentration of at least about 0.001, 0.01, 0.1, 1, 10, 100, 1000, 10000 mg/L. In some cases, the supplemented medium comprises an ammonia concentration of at most about 0.001, 0.01, 0.1, 1, 10, 100, 1000, 10000 mg/L. In some cases, the supplemented medium comprises an ionic strength of at least about 0.001, 0.01, 0.1, 1, 2, 3, 4, or 5 M. In some cases, the supplemented medium comprises an ionic strength of at most about 0.001, 0.01, 0.1, 1, 2, 3, 4, or 5 M.

### Biological Sample

A biological sample can comprise a single sample or a plurality of samples from a species, an individual organism, or a part of an individual organism. In some embodiments, the biological sample can be obtained from an individual organism. In some embodiments, the biological sample can comprise a plurality of samples obtained from a population of organisms. In some embodiments, the biological sample can comprise a gene. In some embodiments, the biological sample can comprise a tissue. In some embodiments, the biological sample can comprise an organ. In some embodiments, the biological sample can be obtained by performing a biopsy. In some embodiments, the biological sample can be obtained by performing a tissue biopsy. In some embodiments, the biological sample can comprise a tumor biopsy. In some embodiments, the biological sample can comprise a liquid biopsy. In some cases, the biological sample may be processed (e.g., lysed, blended, centrifuged, fractionated, etc.). In some cases, the biological sample may comprise media comprising biomolecules secreted by one or more cells. In some cases, the biological sample may be cell-free or substantially cell-free. In some cases, the biological sample may comprise a plurality of biomolecules. In some cases, a plurality of biomolecules may comprise polyamino acids. In some cases, the polyamino acids comprise peptides, proteins, or a combination thereof. In some cases, the plurality of biomolecules may comprise nucleic acids, carbohydrates, polyamino acids, or any combination thereof. A biological sample may comprise a member of any class of biomolecules, where “classes” may refer to any named category that defines a group of biomolecules having a common characteristic (e.g., proteins, nucleic acids, carbohydrates).

In some embodiments, the biological sample can comprise a cell. In some embodiments, a cell can refer to a basic unit of life comprising at least a cellular membrane and genetic material. In some embodiments, a biological sample can comprise a cell of a single-celled organism. In some embodiments, a biological sample can comprise a cell of a multicellular organism. In some embodiments, a biological sample can comprise a bacterial cell. In some embodiments, a biological sample can comprise a fungal cell. In some embodiments, a biological sample can comprise a virus-infected cell. In some embodiments, a biological sample can comprise a mammalian cell. In some embodiments, a biological sample can comprise a human cell. In some embodiments, a biological sample can comprise a specialized cell in a multicellular organism. In some embodiments, a biological sample can comprise a stem cell. In some embodiments, a biological sample can comprise a healthy cell. In some embodiments, a biological sample can comprise a cancerous cell. In some embodiments, a biological sample can comprise a malignant cell. In some embodiments, a biological sample may comprise nucleic acids and various forms thereof. In some embodiments, a biological sample may comprise proteins and various forms thereof. In some cases, a cell is part of a plurality of cells. In some cases, the plurality of cells are cells of a same type. In some cases, the cell is of a tissue sample, an organoid, an immortalized cell line, or any combination thereof. In some cases, the cell is a stem cell. In some cases, the cell is afflicted with an infection or a mutation. In some cases, the cell is a viable cell comprising a cancer cell, an epithelial cell, a bone cell, a muscle cell, a fat cell, a tissue cell, or nerve cell. In some cases, the cancer cell is a biopsied cell of a patient. In some cases, the cell is a eukaryote or a prokaryote. In some cases, the biological sample can comprise a yeast. In some cases. In some cases, the plurality of cells is comprised in a tissue, an organoid, an organism, or a plurality of organisms. In some cases, the cell is derived from an immortalized cell line. In some cases, the cell is a HeLa cell. In some cases, the cell is a stem cell. In some cases, the cell is comprised in a primary cell culture. In some cases, the cell comprises a genetically modified cell. The genetically modified cell may comprise a nucleic acid comprising an engineered or recombinant nucleic acid.

A subject can comprise any living organism. In some embodiments, a subject can be a cell. In some embodiments, a subject can comprise a bacterium, a mammalian cell, a human cell, a fungal cell, a colony of bacteria, a tissue of a mammal, an organ of a mammal, a mammal, a tissue of a human, an organ of a human, a fungus, or any combination thereof. In some embodiments, a subject can comprise a cancer cell, a healthy cell, or both. In some embodiments, a cell can comprise a genetically modified cell.

In some embodiments, a biological sample may comprise a secretome. In some cases, a secretome can refer to factors that are secreted by a cell, a tissue, or an organism to an extracellular space under some defined time and/or conditions. In some cases, the factors may include soluble factors such as proteins, peptides, lipids, extracellular vesicles, or any combination thereof.

In some embodiments, a biological sample may comprise an exosome. In some cases, a secretome may comprise an exosome. In some cases, the biological sample comprises an exosome that has been enriched. In some cases, the biological sample comprises an exosome that has been preferentially enriched. For example, an exosome may be enriched from plasma or a secretome using ultracentrifugation. In some cases, an exosome may comprise least about 100, 200, 300, 400, 500, 600, 700, 800, 900, 1,000, 2,000, 3,000, 4,000, 5,000, 6,000, 7,000, 8,000, 9,000, 10,000, 20,000, 30,000, 30,000, 40,000, 50,000, 60,000, 70,000, 80,000, 90,000, or 100,000 unique biomolecules. In some cases, an exosome may comprise a smaller dynamic range of biomolecules than a human plasma sample. In some cases, an exosome may comprise a dynamic range of at least about 1, 2,3, 4, 5, 6, 7, 8, 9, 10, 11, 12, 13, 14, 15, 16, 17, 18, 19, or 20. In some cases, an exosome may comprise a dynamic range of at most about 1, 2,3, 4, 5, 6, 7, 8, 9, 10, 11, 12, 13, 14, 15, 16, 17, 18, 19, or 20. In some cases, a biomolecule in an exosome may comprise higher visibility or detectability than the same biomolecule not in the exosome. In some cases, at least about 1, 10, 100, or 1000 biomolecules in an exosome may comprise higher visibility or detectability than the same biomolecules not in the same exosome. In some cases, an exosome may be processed. In some cases, the exosome may be processed at least partially by adding a lyse buffer to the one or more exosomes. In some cases, the exosome may be processed at least partially by providing acoustic energy to the one or more exosomes. In some cases, the exosome may be processed at least partially by freezing and thawing the one or more exosomes. In some cases, the exosome may be processed at least partially by heating the one or more exosomes. In some cases, the exosome may be processed at least partially by shearing the one or more exosomes. In some cases, the exosome may be processed at least partially by grinding the one or more exosomes.

In some cases, a cell may secrete a biomolecule via a transmembrane protein. In some cases, a cell may secrete a biomolecule via a porosome. In some cases, a cell may secrete a biomolecule via a vesicle. In some cases, a cell may secrete a biomolecule via an exosome. In some cases, a cell may secrete a biomolecule via a Type 1 secretion system, Type 2 secretion system, Type 3 secretion system, Type 4 secretion system, Type 5 secretion system, Type 6 secretion system, or any combination thereof. In some cases, a cell may secrete conventional secretions, unconventional secretions, Type 1 unconventional secretions, or Type 2 unconventional secretions.

In some cases, a secreted biomolecule may comprise one or more biomarkers, molecular signatures, proteins, exosomes, liposomes, lipids, hormones, glycoproteins, lipoproteins, glycolipids, glycoproteins, peptides, hormones, cholesterols, carbohydrates, apoptotic matter, necrotic matter, nucleic acids, macromolecular assemblies or any combination thereof. In some cases, a secreted biomolecule may comprise an engineered or recombinant biomolecule (e.g., recombinant protein).

### Automated Secretome Assay

In some cases, an automated fluidic system may be used to transfer biological samples. In some cases, the automated fluidic system comprises a microfluidic system. In some cases, the automated fluidic system may be configured to provide supplemented medium at various times. In some cases, the automated fluidic system may be configured to change a composition of a supplemented medium provided at various times. In some cases, the transferring may be performed using one or more fluidic connections and one or more pumps comprised within the loading unit. In some cases, the transferring is performed using one or more pipettes and one or more pumps comprised within the loading unit.

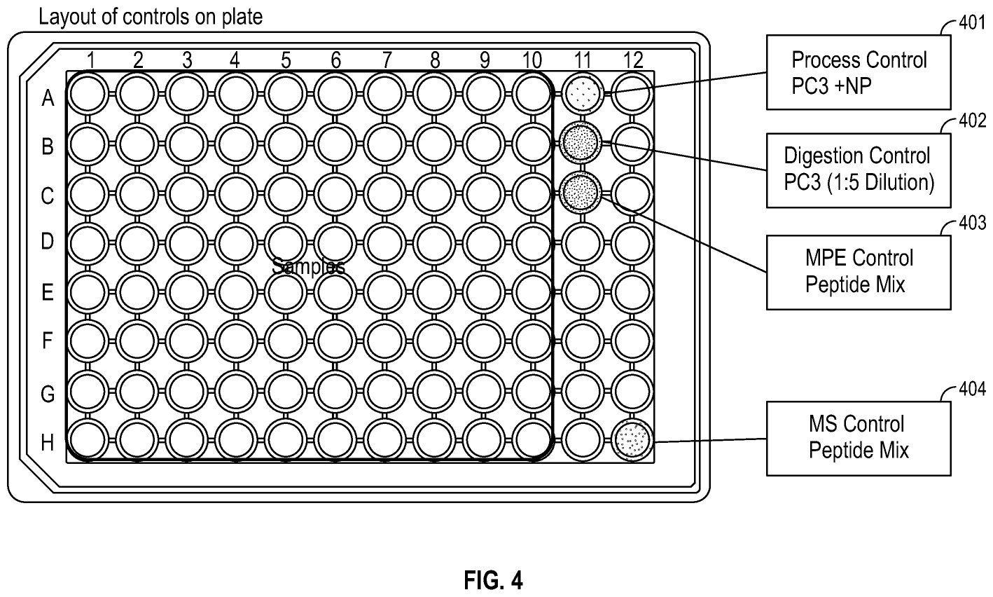

In some cases, the apparatus may comprise a partition containing therein a particle. In some cases, a partition may be chamber. In some cases, a chamber may be partition. FIG. 4 and FIG. 5 show a plurality of partitions, in accordance with some embodiments. In some cases, the apparatus may comprise a single partition (e.g., an Eppendorf tube) for holding a volume of sample or reagents. In some cases, the apparatus may comprise a plurality of partitions (e.g., a 16 well plate, a 96 well plate, a 384 well plate, a plurality of wells in a microwell plate) for holding sample or reagent volumes. In some cases, a partition may comprise a well, a channel (e.g., a microfluidic channel in a microfluidic device), or a compartment. In some cases, a partition may comprise plasticware (e.g., a plastic multi-well plate), a metal structure (e.g., a metal multi-well plate), a carbon material structure (e.g., a carbon composite material multi-well plate), a gel, glassware, or any combination thereof. In some cases, a fluidic channel or chamber may be a microfluidic or nanofluidic channel or chamber. In some cases, a partition may be sealed (e.g., with a operable plastic slip or a pierceable septum) or be sealable (e.g., may comprise a reusable cap or lid). In some cases, a partition may be configured to hold a volume of at least 1 to 10 microliters (μL), at least 5 to 25 μL, at least 20 to 50 μL, at least 40 to 200 μL, at least 100 to 500 μL, at least 200 μL to 1 mL, at least 2 mL, at least 3 mL, or more. In some cases, a partition may be configured to hold a volume of less than about 240 μL, 200 μL, 150 μL, 100 μL, 75 μL, 50 μL, 25 μL, 10 μL, 5 μL, 1 μL, or less. In some cases, a partition may be temperature controlled. In some cases, a partition may be configured to prevent or diminish evaporation. In some cases, a partition may be designed to minimize the influx of ambient light. In some cases, a partition may contain one or more partitions configured to hold control volumes. Non-limiting examples of control volumes which may be used in connection the methods and systems disclosed herein are illustrated in FIG. 4. The control volume(s) may comprise one or more of process control 401, digestion control 402, control peptide mix 403, or mass spectrometry control peptide mix 404, or any combination thereof. In some cases, a plurality of partitions may be grouped by particles, samples, control or any combination thereof. In an example illustrated in FIG. 5, the plurality of partitions comprises 8 rows and 12 columns that can be used with 5 types of particles (e.g., NP1, NP2, NP3, NP4, and NP5). In some cases, each nanoparticle may occupy two columns, and up to 16 biological samples may be deposited. In some cases, each biological sample is labeled as X1, X2, X3, and so forth, until X16. In some cases, there may be two columns for control experiments, wherein each control well in the columns may receive a control particle composition, a control biological sample, or both. In some cases, a control biological sample may comprise a reference composition of biomolecules comprising a predetermined composition. In some cases, a control well may be configured to receive a proteolytic enzyme to quality control the proteolytic enzyme used in an assay. In some cases, each control well may be utilized at a certain step or between steps of an experiment so that an experimental procedure being followed can be troubleshooted. In some cases, particles may be populated in the partitions and then the biological samples may be added in after. In some cases, the biological samples may be populated in the partitions and then the particles may be added in after. In some cases, a subset of the partitions may be grouped by particle or grouped by sample. In some cases, the plurality of partitions may comprise rows for samples and columns for particles. In some cases, the plurality of partitions may be grouped by a specific composition of particles.

In some cases, a partition may comprise a single particle for a single biological sample. In some cases, a partition may comprise a plurality of particles for a single biological sample. In some cases, a partition may comprise a single particle for a plurality of biological samples. In some cases, a partition may comprise a plurality of particles for a plurality of biological samples.

In some cases, the one or more transfer units may be configured to transport a biological sample (e.g., secretome or exosome) to a single partition, or to divide the sample among a plurality of partitions, or to sequentially transfer the sample from one partition to another. For example, a 5 ml sample may be evenly divided between 500 partitions, resulting in separate 10 μl sample volumes. In some cases, a sample may be mixed with reagents within a partition. In some cases, a sample may undergo a dilution within a partition.

In some cases, the apparatus may comprise a magnet configured to apply a magnetic field to the contents of a partition. In some cases, the applied magnetic field may separate magnetic substances from non-magnetic substances within a partition. In some cases, the apparatus may comprise a shaker. In some cases, the substrate may be shaken, vibrated, or sonicated by an instrument.

In some cases, the partition may be operably coupled to the one or more transfer units, for example, as shown in FIG. 8. In some cases, the one or more transfer units may couple temporarily to the partition to transport a portion of a biological sample from the partition 801. In some cases, the one or more transfer units may move in proximity to the partition, make contact with a biological sample in the partition, collect a portion of the biological sample from the partition, and then move away from the partition. In some cases, the one or more transfer units may couple temporarily to the partition to transport the partition 802. In some cases, the one or more transfer units may move in proximity to the partition, couple to the partition, and transfer the partition.

In some cases, the one or more transfer units may be coupled to the partition via fluidic connection. In some cases, the one or more transport units may actuate a pump such that a portion of a biological sample in the partition is transported from the partition to another component in the apparatus.

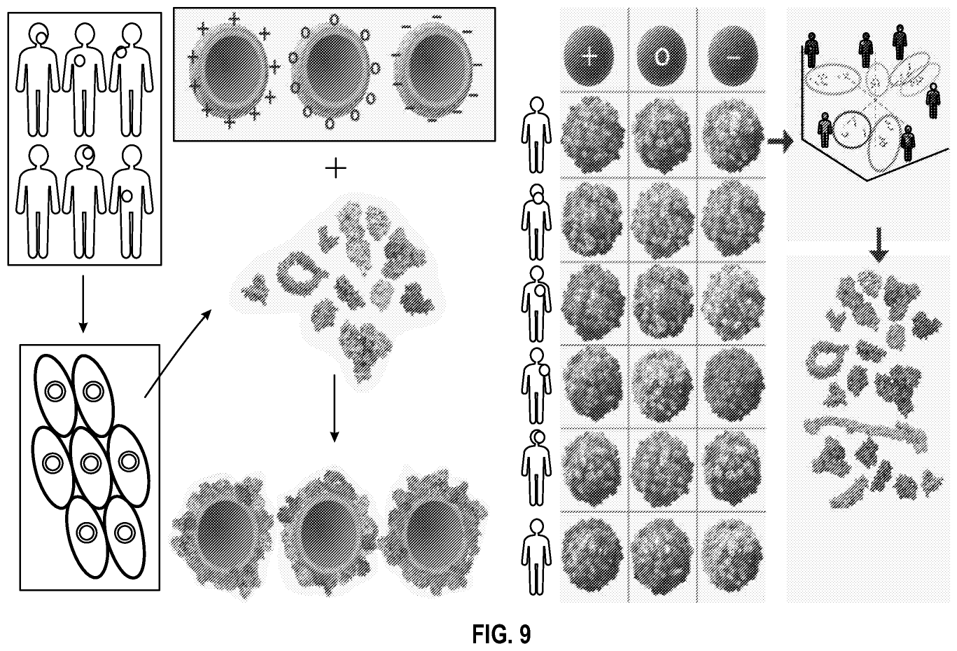

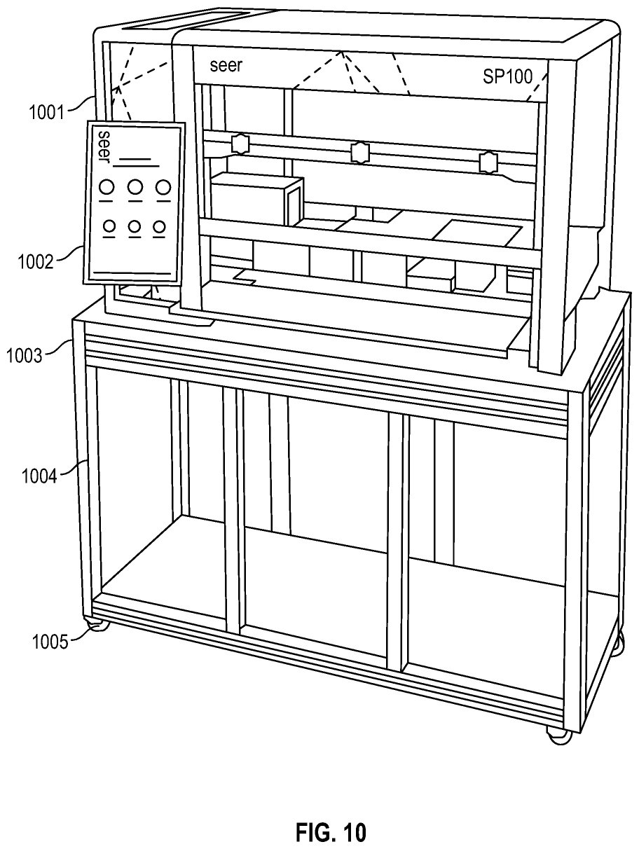

FIG. 10 illustrates an apparatus in accordance with some embodiments. In some cases, the apparatus may comprise a stage 1003. In some cases, components of the apparatus may be coupled to the stage 1003. In some cases, the stage 1003 may comprise one or more supports 1004 coupled thereto. In some cases, the one or more supports 1004 may be configured to move the apparatus. For example, in the apparatus illustrated in FIG. 10, support 1004 are equipped with wheels 1005. In some cases, components of the apparatus may be coupled to the one or more supports 1004. In some cases, the apparatus may comprise a housing 1001. In some cases, components of the apparatus may be disposed inside the housing. In some cases, the apparatus may comprise a display 1002 coupled thereto. In some cases, housing may comprise one or more supports coupled thereto. In some cases, the apparatus may comprise a rail. In some cases, a component of the apparatus may be movably coupled to the rail.

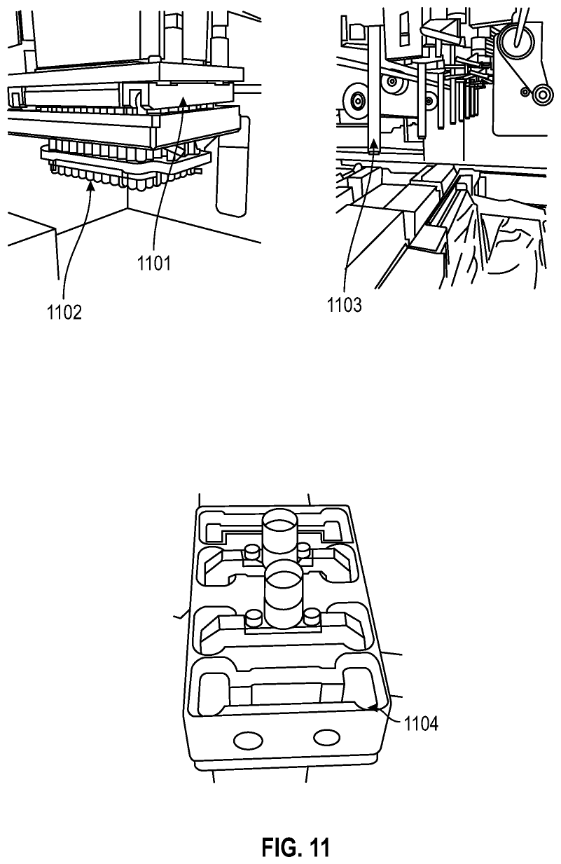

In some cases, the apparatus may comprise one or more transfer units 1101, 1103. In some cases, a transfer unit may be configured to transport liquid samples. FIG. 11 shows transfer units, in accordance with some embodiments. In some cases, a transfer unit may be configured to transport solid samples. In some cases, a transfer unit may comprise a pipette 1102. In some cases, a transfer unit may comprise a plurality of pipettes. In some cases, a transfer unit may comprise a pump. In some cases, a transfer unit may be movable. In some cases, a transfer unit may comprise a rail. In some cases, a transfer unit may comprise a motor configured to move the transfer unit across the rail. In some cases, a transfer unit may comprise a plurality of rails, such that the transfer unit is movable in at least two dimensions. In some cases, a transfer unit may comprise a plurality of rails, such that the transfer unit is movable in at least three dimensions. In some cases, the transfer unit may comprise a robotic arm. In some cases, a transfer unit may comprise a gripper 1104. In some cases, a gripper may be configured to transfer one or more partitions.

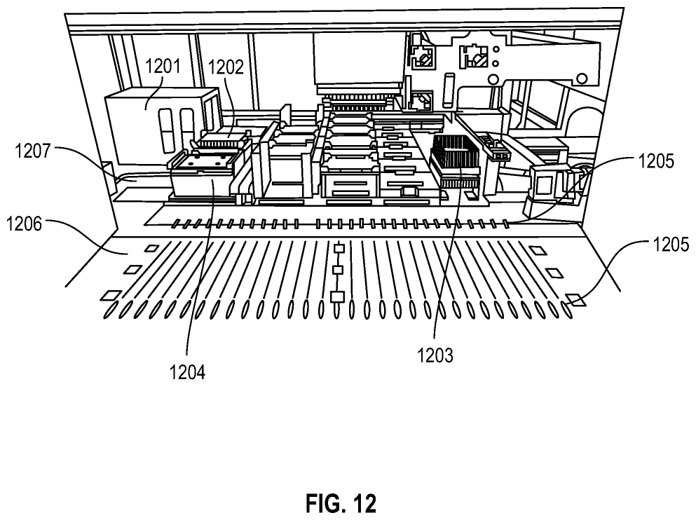

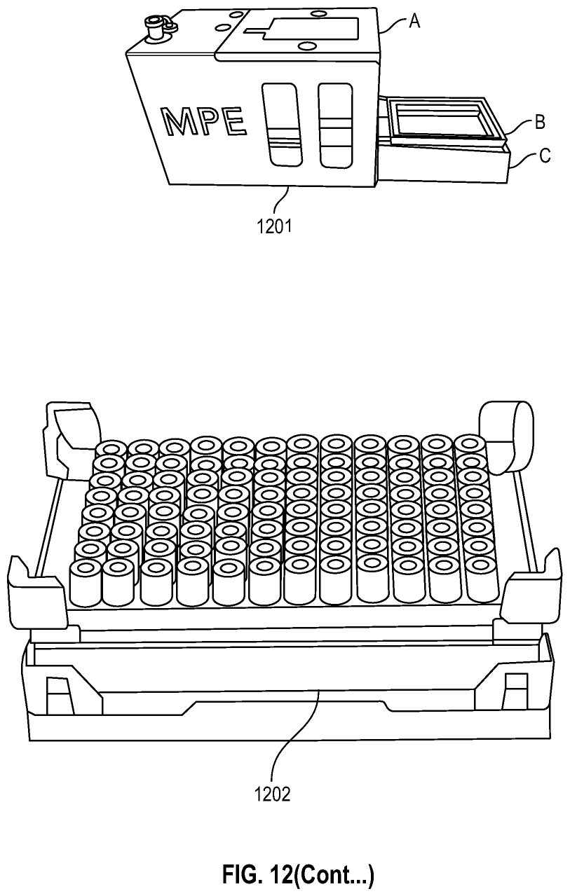

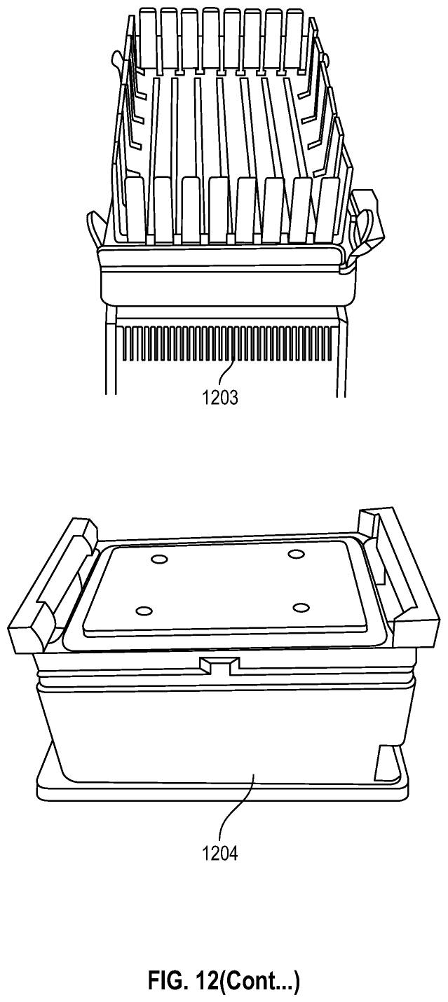

FIG. 12 shows an illustration of apparatus components. In some cases, the apparatus may comprise a filtration system 1201. In some cases, the filtration system may comprise a vacuum. In some cases, the filtration system may comprise a pump. In some cases, the apparatus may comprise a magnetic separation system 1202. In some cases, the magnetic separation system may comprise a magnet. In some cases, the magnetic separation system may be configured to couple with one or more partitions. In some cases, the apparatus may comprise a chiller 1203. In some cases, the apparatus may comprise a heater, shaker, or both 1204. In some cases, the apparatus may comprise rules 1205. In some cases, the apparatus may comprise a working surface 1206. In some cases, the apparatus may comprise a work deck 1207.

In some cases, the sample storage unit may be operably coupled to the one or more transfer units. In some cases, the one or more transfer units may couple temporarily to the sample storage unit to transport a portion of a biological sample from the sample storage unit. In some cases, the one or more transfer units may move in proximity to the sample storage unit, make contact with a biological sample in the sample storage unit, collect a portion of the biological sample from the sample storage unit, and then move away from the sample storage unit, for example as shown in FIG. 6.

In some cases, the one or more transfer units may be coupled to the sample storage unit via fluidic connection, for example as shown in FIG. 7. In some cases, the one or more transport units may actuate a pump such that a portion of a biological sample in the sample storage unit is transported from the sample storage unit to another component in the apparatus.

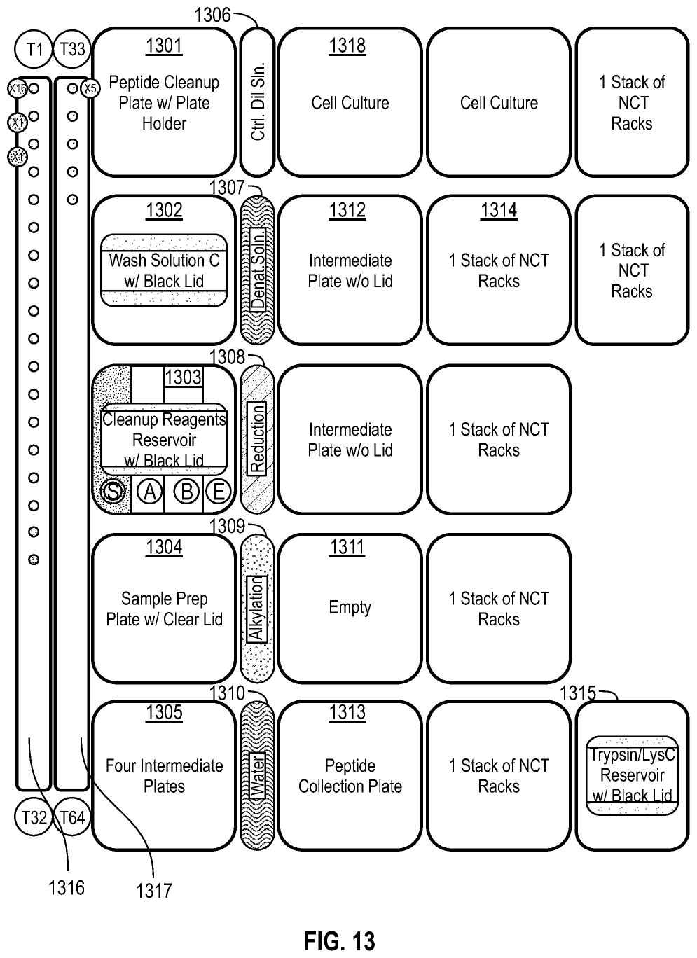

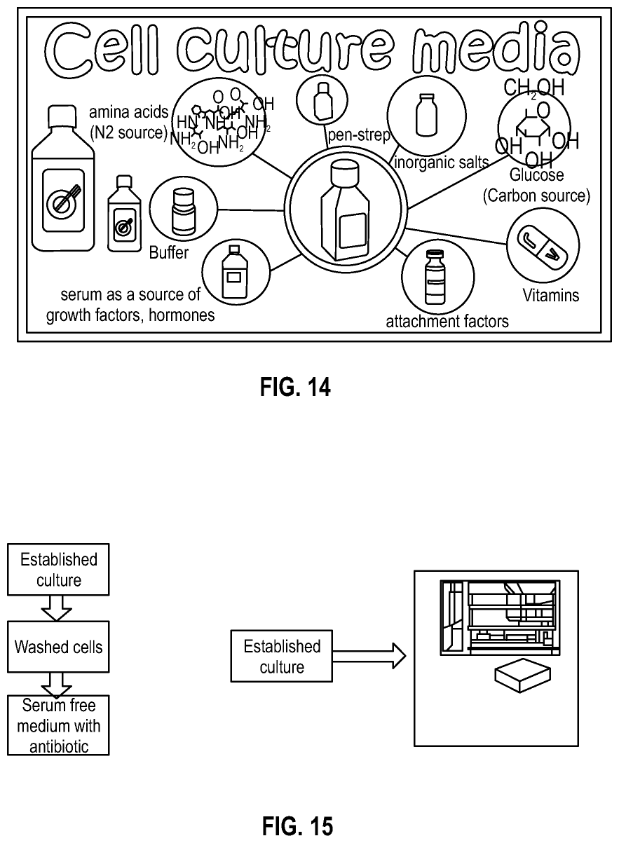

FIG. 13 shows a layout of apparatus components, in accordance with some embodiments. In some cases, the apparatus may comprise a sample storage chamber or well 1316 configured to receive and retain the biological sample. In some cases, the sample storage chamber or well may be configured to receive and retain at least 1, 2, 4, 8, 16, 32, 64, 96, 128, or 256 distinct biological samples. In some cases, the apparatus may comprise a particle storage chamber or well 1317 configured to receive and retain one or more particles. In some cases, the particle storage chamber or well may be configured to receive and retain at least 1, 2, 4, 8, 16, 32, 64, 96, 128, or 256 distinct particles. In some cases, the apparatus may comprise a plurality of plates. In some cases, the apparatus may comprise a cleanup plate 1301, a sample preparation plate 1304, intermediate plate 1305, peptide collection plate 1313, or any combination thereof. In some cases, the apparatus may comprise a plurality of reagent storage chambers or wells. In some cases, the apparatus may comprise a reagent storage chamber or well for a wash solution 1302, cleanup reagents 1303, control dilution solution 1306, denaturation reagent 1307, reduction reagent 1308, alkylation reagent 1309, water 1310, trypsin reagent/lyse reagent 1315, or any combination thereof. In some cases, the apparatus may comprise empty slots 1311 for additional components. In some cases, the apparatus may comprise a rack for pipette tips 1314. In some cases, the apparatus may comprise one or more cell culture chambers 1318.

In some cases, the apparatus may comprise one or sensors. In some cases, the one or more sensors may comprise an optical sensor. In some cases, the optical sensor may comprise a microscope. In some cases, the optical sensor may comprise a camera. In some cases, the optical sensor may be configured to obtain image of a cell. In some cases, the optical sensor may be configured to obtain an image of a cell culture chamber.

### Proteomic Analysis

As used herein, “proteomic analysis”, “protein analysis”, and the like, may refer to any system or method for analyzing proteins in a sample, including the systems and methods disclosed herein. The present disclosure systems and methods for assaying using one or more surface. In some cases, a surface may comprise a surface of a high surface-area material, such as nanoparticles, particles, microparticles, or porous materials. As used herein, a “surface” may refer to a surface for assaying polyamino acids. When a particle composition, physical property, or use thereof is described herein, it shall be understood that a surface of the particle may comprise the same composition, the same physical property, or the same use thereof, in some cases. Similarly, when a surface composition, physical property, or use thereof is described herein, it shall be understood that a particle may comprise the surface to comprise the same composition, the same physical property, or the same use thereof.

Materials for particles and surfaces may include metals, polymers, magnetic materials, and lipids. In some cases, magnetic particles may be iron oxide particles. Examples of metallic materials include any one of or any combination of gold, silver, copper, nickel, cobalt, palladium, platinum, iridium, osmium, rhodium, ruthenium, rhenium, vanadium, chromium, manganese, niobium, molybdenum, tungsten, tantalum, iron, cadmium, or any alloys thereof. In some cases, a particle disclosed herein may be a magnetic particle, such as a superparamagnetic iron oxide nanoparticle (SPION). In some cases, a magnetic particle may be a ferromagnetic particle, a ferrimagnetic particle, a paramagnetic particle, a superparamagnetic particle, or any combination thereof (e.g., a particle may comprise a ferromagnetic material and a ferrimagnetic material).

The present disclosure describes panels of particles or surfaces. In some cases, a panel may comprise more than one distinct surface types. Panels described herein can vary in the number of surface types and the diversity of surface types in a single panel. For example, surfaces in a panel may vary based on size, polydispersity, shape and morphology, surface charge, surface chemistry and functionalization, and base material. In some cases, panels may be incubated with a sample to be analyzed for polyamino acids, polyamino acid concentrations, nucleic acids, nucleic acid concentrations, or any combination thereof. In some cases, polyamino acids in the sample adsorb to distinct surfaces to form one or more adsorption layers of biomolecules. The identity of the biomolecules and concentrations thereof in the one or more adsorption layers may depend on the physical properties of the distinct surfaces and the physical properties of the biomolecules. Thus, each surface type in a panel may have differently adsorbed biomolecules due to adsorbing a different set of biomolecules, different concentrations of a particular biomolecules, or a combination thereof. Each surface type in a panel may have mutually exclusive adsorbed biomolecules or may have overlapping adsorbed biomolecules.

In some cases, panels disclosed herein can be used to identify the number of distinct biomolecules disclosed herein over a wide dynamic range in a given biological sample. For example, a panel may enrich a subset of biomolecules in a sample, which can be identified over a wide dynamic range at which the biomolecules are present in a sample (e.g., a secretome or exosome). In some cases, the enriching may be selective—e.g., biomolecules in the subset may be enriched but biomolecules outside of the subset may not enriched and/or be depleted. In some cases, the subset may comprise proteins having different post-translational modifications. For example, a first particle type in the particle panel may enrich a protein or protein group having a first post-translational modification, a second particle type in the particle panel may enrich the same protein or same protein group having a second post-translational modification, and a third particle type in the particle panel may enrich the same protein or same protein group lacking a post-translational modification. In some cases, the panel including any number of distinct particle types disclosed herein, enriches and identifies a single protein or protein group by binding different domains, sequences, or epitopes of the protein or protein group. For example, a first particle type in the particle panel may enrich a protein or protein group by binding to a first domain of the protein or protein group, and a second particle type in the particle panel may enrich the same protein or same protein group by binding to a second domain of the protein or protein group. In some cases, a panel including any number of distinct particle types disclosed herein, may enrich and identify biomolecules over a dynamic range of at least 5, 6, 7, 8, 9, 10, 15, or 20 magnitudes. In some cases, a panel including any number of distinct particle types disclosed herein, may enrich and identify biomolecules over a dynamic range of at most 5, 6, 7, 8, 9, 10, 15, or 20 magnitudes.

A panel can have more than one surface type. Increasing the number of surface types in a panel can be a method for increasing the number of proteins that can be identified in a given sample.

A particle or surface may comprise a polymer. The polymer may constitute a core material (e.g., the core of a particle may comprise a particle), a layer (e.g., a particle may comprise a layer of a polymer disposed between its core and its shell), a shell material (e.g., the surface of the particle may be coated with a polymer), or any combination thereof. Examples of polymers include any one of or any combination of polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, or polyamines, a polyalkylene glycol (e.g., polyethylene glycol (PEG)), a polyester (e.g., poly(lactide-co-glycolide) (PLGA), polylactic acid, or polycaprolactone), or a copolymer of two or more polymers, such as a copolymer of a polyalkylene glycol (e.g., PEG) and a polyester (e.g., PLGA). The polymer may comprise a cross link. A plurality of polymers in a particle may be phase separated, or may comprise a degree of phase separation.

Examples of lipids that can be used to form the particles or surfaces of the present disclosure include cationic, anionic, and neutrally charged lipids. For example, particles and/or surfaces can be made of any one of or any combination of dioleoylphosphatidylglycerol (DOPG), diacylphosphatidylcholine, diacylphosphatidylethanolamine, ceramide, sphingomyelin, cephalin, cholesterol, cerebrosides and diacylglycerols, dioleoylphosphatidylcholine (DOPC), dimyristoylphosphatidylcholine (DMPC), and dioleoylphosphatidylserine (DOPS), phosphatidylglycerol, cardiolipin, diacylphosphatidylserine, diacylphosphatidic acid, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), lecithin, lysolecithin, phosphatidylethanolamine, lysophosphatidylethanolamine, dioleoylphosphatidylethanolamine (DOPE), dipalmitoyl phosphatidyl ethanolamine (DPPE), dimyristoylphosphoethanolamine (DMPE), distearoyl-phosphatidyl-ethanolamine (DSPE), palmitoyloleoyl-phosphatidylethanolamine (POPE) palmitoyloleoylphosphatidylcholine (POPC), egg phosphatidylcholine (EPC), distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), palmitoyloleyolphosphatidylglycerol (POPG), 16-O-monomethyl PE, 16-O-dimethyl PE, 18-1-trans PE, palmitoyloleoyl-phosphatidylethanolamine (POPE), 1-stearoyl-2-oleoyl-phosphatidyethanolamine (SOPE), phosphatidylserine, phosphatidylinositol, sphingomyelin, cephalin, cardiolipin, phosphatidic acid, cerebrosides, dicetylphosphate, cholesterol, and any combination thereof.

A particle panel may comprise a combination of particles with silica and polymer surfaces. For example, a particle panel may comprise a SPION coated with a thin layer of silica, a SPION coated with poly(dimethyl aminopropyl methacrylamide) (PDMAPMA), and a SPION coated with poly(ethylene glycol) (PEG). A particle panel consistent with the present disclosure could also comprise two or more particles selected from the group consisting of silica coated SPION, an N-(3-Trimethoxysilylpropyl) diethylenetriamine coated SPION, a PDMAPMA coated SPION, a carboxyl-functionalized polyacrylic acid coated SPION, an amino surface functionalized SPION, a polystyrene carboxyl functionalized SPION, a silica particle, and a dextran coated SPION. A particle panel consistent with the present disclosure may also comprise two or more particles selected from the group consisting of a surfactant free carboxylate particle, a carboxyl functionalized polystyrene particle, a silica coated particle, a silica particle, a dextran coated particle, an oleic acid coated particle, a boronated nanopowder coated particle, a PDMAPMA coated particle, a Poly(glycidyl methacrylate-benzylamine) coated particle, and a Poly(N-[3-(Dimethylamino) propyl]methacrylamide-co-[2-(methacryloyloxy)ethyl]dimethyl-(3-sulfopropyl) ammonium hydroxide, P (DMAPMA-co-SBMA) coated particle. A particle panel consistent with the present disclosure may comprise silica-coated particles, N-(3-Trimethoxysilylpropyl) diethylenetriamine coated particles, poly(N-(3-(dimethylamino) propyl) methacrylamide) (PDMAPMA)-coated particles, phosphate-sugar functionalized polystyrene particles, amine functionalized polystyrene particles, polystyrene carboxyl functionalized particles, ubiquitin functionalized polystyrene particles, dextran coated particles, or any combination thereof.

A particle panel consistent with the present disclosure may comprise a silica functionalized particle, an amine functionalized particle, a silicon alkoxide functionalized particle, a carboxylate functionalized particle, and a benzyl or phenyl functionalized particle. A particle panel consistent with the present disclosure may comprise a silica functionalized particle, an amine functionalized particle, a silicon alkoxide functionalized particle, a polystyrene functionalized particle, and a saccharide functionalized particle. A particle panel consistent with the present disclosure may comprise a silica functionalized particle, an N-(3-Trimethoxysilylpropyl) diethylenetriamine functionalized particle, a PDMAPMA functionalized particle, a dextran functionalized particle, and a polystyrene carboxyl functionalized particle. A particle panel consistent with the present disclosure may comprise 5 particles including a silica functionalized particle, an amine functionalized particle, a silicon alkoxide functionalized particle.

Distinct surfaces or distinct particles of the present disclosure may differ by one or more physicochemical property. The one or more physicochemical property is selected from the group consisting of: composition, size, surface charge, hydrophobicity, hydrophilicity, roughness, density surface functionalization, surface topography, surface curvature, porosity, core material, shell material, shape, and any combination thereof. The surface functionalization may comprise a macromolecular functionalization, a small molecule functionalization, or any combination thereof. A small molecule functionalization may comprise an aminopropyl functionalization, amine functionalization, boronic acid functionalization, carboxylic acid functionalization, alkyl group functionalization, N-succinimidyl ester functionalization, monosaccharide functionalization, phosphate sugar functionalization, sulfurylated sugar functionalization, ethylene glycol functionalization, streptavidin functionalization, methyl ether functionalization, trimethoxysilylpropyl functionalization, silica functionalization, triethoxylpropylaminosilane functionalization, thiol functionalization, PCP functionalization, citrate functionalization, lipoic acid functionalization, ethyleneimine functionalization. A particle panel may comprise a plurality of particles with a plurality of small molecule functionalizations selected from the group consisting of silica functionalization, trimethoxysilylpropyl functionalization, dimethylamino propyl functionalization, phosphate sugar functionalization, amine functionalization, and carboxyl functionalization.

A small molecule functionalization may comprise a polar functional group. Non-limiting examples of polar functional groups comprise carboxyl group, a hydroxyl group, a thiol group, a cyano group, a nitro group, an ammonium group, an imidazolium group, a sulfonium group, a pyridinium group, a pyrrolidinium group, a phosphonium group or any combination thereof. In some embodiments, the functional group is an acidic functional group (e.g., sulfonic acid group, carboxyl group, and the like), a basic functional group (e.g., amino group, cyclic secondary amino group (such as pyrrolidyl group and piperidyl group), pyridyl group, imidazole group, guanidine group, etc.), a carbamoyl group, a hydroxyl group, an aldehyde group and the like.

A small molecule functionalization may comprise an ionic or ionizable functional group. Non-limiting examples of ionic or ionizable functional groups comprise an ammonium group, an imidazolium group, a sulfonium group, a pyridinium group, a pyrrolidinium group, a phosphonium group. A small molecule functionalization may comprise a polymerizable functional group. Non-limiting examples of the polymerizable functional group include a vinyl group and a (meth)acrylic group. In some embodiments, the functional group is pyrrolidyl acrylate, acrylic acid, methacrylic acid, acrylamide, 2-(dimethylamino)ethyl methacrylate, hydroxyethyl methacrylate and the like.

A surface functionalization may comprise a charge. For example, a particle can be functionalized to carry a net neutral surface charge, a net positive surface charge, a net negative surface charge, or a zwitterionic surface. Surface charge can be a determinant of the types of biomolecules collected on a particle. Accordingly, optimizing a particle panel may comprise selecting particles with different surface charges, which may not only increase the number of different proteins collected on a particle panel, but also increase the likelihood of identifying a biological state of a sample. A particle panel may comprise a positively charged particle and a negatively charged particle. A particle panel may comprise a positively charged particle and a neutral particle. A particle panel may comprise a positively charged particle and a zwitterionic particle. A particle panel may comprise a neutral particle and a negatively charged particle. A particle panel may comprise a neutral particle and a zwitterionic particle. A particle panel may comprise a negative particle and a zwitterionic particle. A particle panel may comprise a positively charged particle, a negatively charged particle, and a neutral particle. A particle panel may comprise a positively charged particle, a negatively charged particle, and a zwitterionic particle. A particle panel may comprise a positively charged particle, a neutral particle, and a zwitterionic particle. A particle panel may comprise a negatively charged particle, a neutral particle, and a zwitterionic particle.

A particle may comprise a single surface such as a specific small molecule, or a plurality of surface functionalizations, such as a plurality of different small molecules. Surface functionalization can influence the composition of a particle's biomolecule corona. Such surface functionalization can include small molecule functionalization or macromolecular functionalization. A surface functionalization may be coupled to a particle material such as a polymer, metal, metal oxide, inorganic oxide (e.g., silicon dioxide), or another surface functionalization.

A surface functionalization may comprise a binding molecule. The binding molecule may be a small molecule, an oligomer, or a macromolecule. The binding molecule may comprise an binding specificity for a group or class of analytes (e.g., a plurality of saccharides or a class of proteins). A binding molecule may comprise a moderate binding specificity for the group or class of analytes. Conversely, a binding molecule may comprise a dis-affinity for a group or class of analytes, disfavoring binding of these species relative to the same particle lacking the binding molecule. For example, a binding molecule may comprise a negative charge distribution which repels negatively charged nucleic acids, thereby disfavoring their binding.

A binding molecule may comprise a peptide. Peptides are an extensive and diverse set of biomolecules which may comprise a wide range of physical and chemical properties. Depending on its composition, sequence, and chemical modification, a peptide may be hydrophilic, hydrophobic, amphiphilic, lipophilic, lipophobic, positively charged, negatively charged, zwitterionic, neutral, chaotropic, antichaotropic, reactive, redox active, inert, acidic, basic, rigid, flexible, or any combination thereof. Accordingly, a peptide surface functionalization may confer a range of physicochemical properties to a particle. A particle may comprise a single peptide surface functionalization or a plurality of peptide surface functionalizations. A single peptide surface functionalization may comprise a plurality of identical or sequence-sharing peptides bound to a particle in a uniform fashion.

A surface functionalization may comprise a small molecule functionalization, a macromolecular functionalization, or a combination of two or more such functionalizations. In some cases, a macromolecular functionalization may comprise a biomacromolecule, such as a protein or a polynucleotide (e.g., a 100-mer DNA molecule). A macromolecular functionalization may be comprise a protein, polynucleotide, or polysaccharide, or may be comparable in size to any of the aforementioned classes of species. In some cases, A surface functionalization may comprise an ionizable moiety. In some cases, a surface functionalization may comprise pKa of at least about 1, 2, 3, 4, 5, 6, 7, 8, 9, 10, 11, 12, 13, or 14. In some cases, a surface functionalization may comprise pKa of at most about 1, 2, 3, 4, 5, 6, 7, 8, 9, 10, 11, 12, 13, or 14. In some cases, a small molecule functionalization may comprise a small organic molecule such as an alcohol (e.g., octanol), an amine, an alkane, an alkene, an alkyne, a heterocycle (e.g., a piperidinyl group), a heteroaromatic group, a thiol, a carboxylate, a carbonyl, an amide, an ester, a thioester, a carbonate, a thiocarbonate, a carbamate, a thiocarbamate, a urea, a thiourea, a halogen, a sulfate, a phosphate, a monosaccharide, a disaccharide, a lipid, or any combination thereof. For example, a small molecule functionalization may comprise a phosphate sugar, a sugar acid, or a sulfurylated sugar.

In some cases, a macromolecular functionalization may comprise a specific form of attachment to a particle. In some cases, a macromolecule may be tethered to a particle via a linker. In some cases, the linker may hold the macromolecule close to the particle, thereby restricting its motion and reorientation relative to the particle, or may extend the macromolecule away from the particle. In some cases, the linker may be rigid (e.g., a polyolefin linker) or flexible (e.g., a nucleic acid linker). In some cases, a linker may be at least about 0.5, 1, 2, 3, 4, 5, 6, 7, 8, 9, 10, 15, 20, 25, or 30 nm in length. In some cases, a linker may be at most about 0.5, 1, 2, 3, 4, 5, 6, 7, 8, 9, 10, 15, 20, 25, or 30 nm in length. As such, a surface functionalization on a particle may project beyond a primary corona associated with the particle. In some cases, a surface functionalization may also be situated beneath or within a biomolecule corona that forms on the particle surface. In some cases, a macromolecule may be tethered at a specific location, such as at a protein's C-terminus, or may be tethered at a number of possible sites. For example, a peptide may be covalent attached to a particle via any of its surface exposed lysine residues.

In some embodiments, a macromolecule can be modified with a peptide. In some embodiments, the macromolecule comprises a thiol or azide. In some embodiments, a surface comprises the macromolecule modified with a peptide immobilized to a surface. In some embodiments, the macromolecule is covalently coupled to the surface. In some embodiments, the macromolecule is electrostatically coupled to the surface. In some embodiments, the macromolecule is coupled to the surface through a polymerization event. In some embodiments, the polymerization event comprises a reaction with a vinyl group on the surface.

In some embodiments, macromolecules modified with peptides can be immobilized on surfaces for identification, binding, or enrichment of biomolecules (e.g., proteins). In some embodiments, a surface can comprise a macromolecule modified with a peptide, wherein the peptide comprises a binding site, and a protein interacting with the peptide at the binding site. In some embodiments, a biological sample can be contacted with a surface comprising the macromolecule modified with a peptide, wherein the peptides are configured to bind to a protein, which can release the plurality of biomolecules from the surface.

In some cases, a particle may be contacted with a biological sample (e.g., a biofluid) to form a biomolecule corona. In some cases, a biomolecule corona may comprise at least two biomolecules that do not share a common binding motif. The particle and biomolecule corona may be separated from the biological sample, for example by centrifugation, magnetic separation, filtration, or gravitational separation. The particle types and biomolecule corona may be separated from the biological sample using a number of separation techniques. Non-limiting examples of separation techniques include comprises magnetic separation, column-based separation, filtration, spin column-based separation, centrifugation, ultracentrifugation, density or gradient-based centrifugation, gravitational separation, or any combination thereof. A protein corona analysis may be performed on the separated particle and biomolecule corona. A protein corona analysis may comprise identifying one or more proteins in the biomolecule corona, for example by mass spectrometry. In some cases, a single particle type may be contacted with a biological sample. In some cases, a plurality of particle types may be contacted to a biological sample. In some cases, the plurality of particle types may be combined and contacted to the biological sample in a single sample volume. In some cases, the plurality of particle types may be sequentially contacted to a biological sample and separated from the biological sample prior to contacting a subsequent particle type to the biological sample. In some cases, adsorbed biomolecules on the particle may have compressed (e.g., smaller) dynamic range compared to a given original biological sample.

In some cases, the particles of the present disclosure may be used to serially interrogate a sample by incubating a first particle type with the sample to form a biomolecule corona on the first particle type, separating the first particle type, incubating a second particle type with the sample to form a biomolecule corona on the second particle type, separating the second particle type, and repeating the interrogating (by incubation with the sample) and the separating for any number of particle types. In some cases, the biomolecule corona on each particle type used for serial interrogation of a sample may be analyzed by protein corona analysis. The biomolecule content of the supernatant may be analyzed following serial interrogation with one or more particle types.

In some cases, a method of the present disclosure may identify a large number of unique biomolecules (e.g., proteins) in a biological sample (e.g., a biofluid). In some cases, a surface disclosed herein may be incubated with a biological sample to adsorb at least 1000, 2000, 3000, 4000, 5000, 6000, 7000, 8000, 9000, or 10000 unique biomolecules. In some cases, a surface disclosed herein may be incubated with a biological sample to adsorb at most 1000, 2000, 3000, 4000, 5000, 6000, 7000, 8000, 9000, or 10000 unique biomolecules. In some cases, a surface disclosed herein may be incubated with a biological sample to adsorb at least 1000, 2000, 3000, 4000, 5000, 6000, 7000, 8000, 9000, or 10000 unique biomolecule groups. In some cases, a surface disclosed herein may be incubated with a biological sample to adsorb at most 1000, 2000, 3000, 4000, 5000, 6000, 7000, 8000, 9000, or 10000 unique biomolecule groups. In some cases, several different types of surfaces can be used, separately or in combination, to identify large numbers of proteins in a particular biological sample. In other words, surfaces can be multiplexed in order to bind and identify large numbers of biomolecules in a biological sample.

In some cases, a method of the present disclosure may identify a large number of unique proteoforms in a biological sample. In some cases, a method may identify at least about 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 unique proteoforms. In some cases, a method may identify at most about 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 unique proteoforms. In some cases, a surface disclosed herein may be incubated with a biological sample to adsorb at least 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 unique proteoforms. In some cases, a surface disclosed herein may be incubated with a biological sample to adsorb at most 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 unique proteoforms. In some cases, several different types of surfaces can be used, separately or in combination, to identify large numbers of proteins in a particular biological sample. In other words, surfaces can be multiplexed in order to bind and identify large numbers of biomolecules in a biological sample.

Biomolecules collected on particles may be subjected to further analysis. In some cases, a method may comprise collecting a biomolecule corona or a subset of biomolecules from a biomolecule corona. In some cases, the collected biomolecule corona or the collected subset of biomolecules from the biomolecule corona may be subjected to further particle-based analysis (e.g., particle adsorption). In some cases, the collected biomolecule corona or the collected subset of biomolecules from the biomolecule corona may be purified or fractionated (e.g., by a chromatographic method). In some cases, the collected biomolecule corona or the collected subset of biomolecules from the biomolecule corona may be analyzed (e.g., by mass spectrometry).

In some cases, the panels disclosed herein can be used to identify a number of proteins, peptides, protein groups, or protein classes using a protein analysis workflow described herein (e.g., a protein corona analysis workflow). In some cases, protein analysis may comprise contacting a sample to distinct surface types (e.g., a particle panel), forming adsorbed biomolecule layers on the distinct surface types, and identifying the biomolecules in the adsorbed biomolecule layers (e.g., by mass spectrometry). Feature intensities, as disclosed herein, may refer to the intensity of a discrete spike (“feature”) seen on a plot of mass to charge ratio versus intensity from a mass spectrometry run of a sample. In some cases, these features can correspond to variably ionized fragments of peptides and/or proteins. In some cases, using the data analysis methods described herein, feature intensities can be sorted into protein groups. In some cases, protein groups may refer to two or more proteins that are identified by a shared peptide sequence. In some cases, a protein group can refer to one protein that is identified using a unique identifying sequence. For example, if in a sample, a peptide sequence is assayed that is shared between two proteins (Protein 1: XYZZX and Protein 2: XYZYZ), a protein group could be the “XYZ protein group” having two members (protein 1 and protein 2). In some cases, if the peptide sequence is unique to a single protein (Protein 1), a protein group could be the “ZZX” protein group having one member (Protein 1). In some cases, each protein group can be supported by more than one peptide sequence. In some cases, protein detected or identified according to the instant disclosure can refer to a distinct protein detected in the sample (e.g., distinct relative other proteins detected using mass spectrometry). In some cases, analysis of proteins present in distinct coronas corresponding to the distinct surface types in a panel yields a high number of feature intensities. In some cases, this number decreases as feature intensities are processed into distinct peptides, further decreases as distinct peptides are processed into distinct proteins, and further decreases as peptides are grouped into protein groups (two or more proteins that share a distinct peptide sequence).

In some cases, the methods disclosed herein include isolating one or more particle types from a sample or from more than one sample (e.g., a biological sample or a serially interrogated sample). The particle types can be rapidly isolated or separated from the sample using a magnet. Moreover, multiple samples that are spatially isolated can be processed in parallel. In some cases, the methods disclosed herein provide for isolating or separating a particle type from unbound protein in a sample. In some cases, a particle type may be separated by a variety of means, including but not limited to magnetic separation, centrifugation, filtration, or gravitational separation. In some cases, particle panels may be incubated with a plurality of spatially isolated samples, wherein each spatially isolated sample is in a well in a well plate (e.g., a 96-well plate). In some cases, the particle in each of the wells of the well plate can be separated from unbound protein present in the spatially isolated samples by placing the entire plate on a magnet. In some cases, this simultaneously pulls down the superparamagnetic particles in the particle panel. In some cases, the supernatant in each sample can be removed to remove the unbound protein. In some cases, these steps (incubate, pull down) can be repeated to effectively wash the particles, thus removing residual background unbound protein that may be present in a sample.

In some cases, the systems and methods disclosed herein may also elucidate protein classes or interactions of the protein classes. In some cases, a protein class may comprise a set of proteins that share a common function (e.g., amine oxidases or proteins involved in angiogenesis); proteins that share common physiological, cellular, or subcellular localization (e.g., peroxisomal proteins or membrane proteins); proteins that share a common cofactor (e.g., heme or flavin proteins); proteins that correspond to a particular biological state (e.g., hypoxia related proteins); proteins containing a particular structural motif (e.g., a cupin fold); proteins that are functionally related (e.g., part of a same metabolic pathway); or proteins bearing a post-translational modification (e.g., ubiquitinated or citrullinated proteins). In some cases, a protein class may contain at least 2 proteins, 5 proteins, 10 proteins, 20 proteins, 40 proteins, 60 proteins, 80 proteins, 100 proteins, 150 proteins, 200 proteins, or more.

In some cases, the proteomic data of the biological sample can be identified, measured, and quantified using a number of different analytical techniques. For example, proteomic data can be generated using SDS-PAGE or any gel-based separation technique. In some cases, peptides and proteins can also be identified, measured, and quantified using an immunoassay, such as ELISA. In some cases, proteomic data can be identified, measured, and quantified using mass spectrometry, high performance liquid chromatography, LC-MS/MS, Edman Degradation, immunoaffinity techniques, and other protein separation techniques.

In some cases, an assay may comprise protein collection of particles, protein digestion, and mass spectrometric analysis (e.g., MS, LC-MS, LC-MS/MS). In some cases, the digestion may comprise chemical digestion, such as by cyanogen bromide or 2-Nitro-5-thiocyanatobenzoic acid (NTCB). In some cases, the digestion may comprise enzymatic digestion, such as by trypsin or pepsin. In some cases, the digestion may comprise enzymatic digestion by a plurality of proteases. In some cases, the digestion may comprise a protease selected from among the group consisting of trypsin, chymotrypsin, Glu C, Lys C, elastase, subtilisin, proteinase K, thrombin, factor X, Arg C, papaine, Asp N, thermolysine, pepsin, aspartyl protease, cathepsin D, zinc mealloprotease, glycoprotein endopeptidase, proline, aminopeptidase, prenyl protease, caspase, kex2 endoprotease, or any combination thereof. In some cases, the digestion may cleave peptides at random positions. In some cases, the digestion may cleave peptides at a specific position (e.g., at methionines) or sequence (e.g., glutamate-histidine-glutamate). In some cases, the digestion may enable similar proteins to be distinguished. For example, an assay may resolve 8 distinct proteins as a single protein group with a first digestion method, and as 8 separate proteins with distinct signals with a second digestion method. In some cases, the digestion may generate an average peptide fragment length of 8 to 15 amino acids. In some cases, the digestion may generate an average peptide fragment length of 12 to 18 amino acids. In some cases, the digestion may generate an average peptide fragment length of 15 to 25 amino acids. In some cases, the digestion may generate an average peptide fragment length of 20 to 30 amino acids. In some cases, the digestion may generate an average peptide fragment length of 30 to 50 amino acids.

In some cases, an assay may rapidly generate biological samples for analysis. In some cases, the biological samples may comprise proteolytic peptides. In some cases, beginning with an input biological sample (e.g., a buccal or nasal smear, plasma, secretome, or tissue), a method of the present disclosure may generate the biological samples in less than about 1, 2, 3,4, 5, 6, 7, 8, 12, 16, 20, 24, or 48 hours. In some cases, beginning with an input biological sample (e.g., a buccal or nasal smear, plasma, secretome, or tissue), a method of the present disclosure may generate the biological samples in less than about 1, 2, 3,4, 5, 6, 7, 8, 12, 16, 20, 24, or 48 hours.

In some cases, an assay may rapidly generate and analyze proteomic data. In some cases, beginning with an input biological sample (e.g., a buccal or nasal smear, plasma, or tissue), a method of the present disclosure may generate and obtain proteomic data in less than about 1, 2, 3,4, 5, 6, 7, 8, 12, 16, 20, 24, or 48 hours. In some cases, beginning with an input biological sample (e.g., a buccal or nasal smear, plasma, or tissue), a method of the present disclosure may generate and analyze proteomic data in less than about 1, 2, 3,4, 5, 6, 7, 8, 12, 16, 20, 24, or 48 hours. In some cases, the analyzing may comprise identifying a protein group. In some cases, the analyzing may comprise identifying a protein class. In some cases, the analyzing may comprise quantifying an abundance of a biomolecule, a peptide, a protein, protein group, or a protein class. In some cases, the analyzing may comprise identifying a ratio of abundances of two biomolecules, peptides, proteins, protein groups, or protein classes. In some cases, the analyzing may comprise identifying a biological state.

An example of a particle type of the present disclosure may be a carboxylate (Citrate) superparamagnetic iron oxide nanoparticle (SPION), a phenol-formaldehyde coated SPION, a silica-coated SPION, a polystyrene coated SPION, a carboxylated poly(styrene-co-methacrylic acid) coated SPION, a N-(3-Trimethoxysilylpropyl) diethylenetriamine coated SPION, a poly(N-(3-(dimethylamino) propyl) methacrylamide) (PDMAPMA)-coated SPION, a 1,2,4,5-Benzenetetracarboxylic acid coated SPION, a poly(Vinylbenzyltrimethylammonium chloride) (PVBTMAC) coated SPION, a carboxylate, PAA coated SPION, a poly(oligo (ethylene glycol) methyl ether methacrylate) (POEGMA)-coated SPION, a carboxylate microparticle, a polystyrene carboxyl functionalized particle, a carboxylic acid coated particle, a silica particle, a carboxylic acid particle of about 150 nm in diameter, an amino surface microparticle of about 0.4-0.6 μm in diameter, a silica amino functionalized microparticle of about 0.1-0.39 μm in diameter, a Jeffamine surface particle of about 0.1-0.39 μm in diameter, a polystyrene microparticle of about 2.0-2.9 μm in diameter, a silica particle, a carboxylated particle with an original coating of about 50 nm in diameter, a particle coated with a dextran based coating of about 0.13 μm in diameter, or a silica silanol coated particle with low acidity. In some cases, a particle may lack functionalized specific binding moieties for specific binding on its surface. In some cases, a particle may lack functionalized proteins for specific binding on its surface. In some cases, a surface functionalized particle does not comprise an antibody or a T cell receptor, a chimeric antigen receptor, a receptor protein, or a variant or fragment thereof. In some cases, the ratio between surface area and mass can be a determinant of a particle's properties. A particle of the present disclosure may be a nanoparticle. A nanoparticle of the present disclosure may be from about 10 nm to about 1000 nm in diameter. For example, the nanoparticles disclosed herein can be at least 10 nm, at least 100 nm, at least 200 nm, at least 300 nm, at least 400 nm, at least 500 nm, at least 600 nm, at least 700 nm, at least 800 nm, at least 900 nm, from 10 nm to 50 nm, from 50 nm to 100 nm, from 100 nm to 150 nm, from 150 nm to 200 nm, from 200 nm to 250 nm, from 250 nm to 300 nm, from 300 nm to 350 nm, from 350 nm to 400 nm, from 400 nm to 450 nm, from 450 nm to 500 nm, from 500 nm to 550 nm, from 550 nm to 600 nm, from 600 nm to 650 nm, from 650 nm to 700 nm, from 700 nm to 750 nm, from 750 nm to 800 nm, from 800 nm to 850 nm, from 850 nm to 900 nm, from 100 nm to 300 nm, from 150 nm to 350 nm, from 200 nm to 400 nm, from 250 nm to 450 nm, from 300 nm to 500 nm, from 350 nm to 550 nm, from 400 nm to 600 nm, from 450 nm to 650 nm, from 500 nm to 700 nm, from 550 nm to 750 nm, from 600 nm to 800 nm, from 650 nm to 850 nm, from 700 nm to 900 nm, or from 10 nm to 900 nm in diameter. A nanoparticle may be less than 1000 nm in diameter. A particle of the present disclosure may be a microparticle. A microparticle may be a particle that is from about 1 μm to about 1000 μm in diameter. For example, the microparticles disclosed here can be at least 1 μm, at least 10 μm, at least 100 μm, at least 200 μm, at least 300 μm, at least 400 μm, at least 500 μm, at least 600 μm, at least 700 μm, at least 800 μm, at least 900 μm, from 10 μm to 50 μm, from 50 μm to 100 μm, from 100 μm to 150 μm, from 150 μm to 200 μm, from 200 μm to 250 μm, from 250 μm to 300 μm, from 300 μm to 350 μm, from 350 μm to 400 μm, from 400 μm to 450 μm, from 450 μm to 500 μm, from 500 μm to 550 μm, from 550 μm to 600 μm, from 600 μm to 650 μm, from 650 μm to 700 μm, from 700 μm to 750 μm, from 750 μm to 800 μm, from 800 μm to 850 μm, from 850 μm to 900 μm, from 100 μm to 300 μm, from 150 μm to 350 μm, from 200 μm to 400 μm, from 250 μm to 450 μm, from 300 μm to 500 μm, from 350 μm to 550 μm, from 400 μm to 600 μm, from 450 μm to 650 μm, from 500 μm to 700 μm, from 550 μm to 750 μm, from 600 μm to 800 μm, from 650 μm to 850 μm, from 700 μm to 900 μm, or from 10 μm to 900 μm in diameter. A microparticle may be less than 1000 μm in diameter. The particles disclosed herein can have surface area to mass ratios of 3 to 30 cm²/mg, 5 to 50 cm²/mg, 10 to 60 cm²/mg, 15 to 70 cm²/mg, 20 to 80 cm²/mg, 30 to 100 cm²/mg, 35 to 120 cm²/mg, 40 to 130 cm²/mg, 45 to 150 cm²/mg, 50 to 160 cm²/mg, 60 to 180 cm²/mg, 70 to 200 cm²/mg, 80 to 220 cm²/mg, 90 to 240 cm²/mg, 100 to 270 cm²/mg, 120 to 300 cm²/mg, 200 to 500 cm²/mg, 10 to 300 cm²/mg, 1 to 3000 cm²/mg, 20 to 150 cm²/mg, 25 to 120 cm²/mg, or from 40 to 85 cm²/mg. Small particles (e.g., with diameters of 50 nm or less) can have significantly higher surface area to mass ratios, stemming in part from the higher order dependence on diameter by mass than by surface area. In some cases (e.g., for small particles), the particles can have surface area to mass ratios of 200 to 1000 cm²/mg, 500 to 2000 cm²/mg, 1000 to 4000 cm²/mg, 2000 to 8000 cm²/mg, or 4000 to 10000 cm²/mg. In some cases (e.g., for large particles), the particles can have surface area to mass ratios of 1 to 3 cm²/mg, 0.5 to 2 cm²/mg, 0.25 to 1.5 cm²/mg, or 0.1 to 1 cm²/mg. A particle may comprise a wide array of physical properties. A physical property of a particle may include composition, size, surface charge, hydrophobicity, hydrophilicity, amphipathicity, surface functionality, surface topography, surface curvature, porosity, core material, shell material, shape, zeta potential, and any combination thereof. A particle may have a core-shell structure. In some cases, a core material may comprise metals, polymers, magnetic materials, paramagnetic materials, oxides, and/or lipids. In some cases, a shell material may comprise metals, polymers, magnetic materials, oxides, and/or lipids.

### Proteomic Information

In some cases, proteomic information or data can refer to information about substances comprising a peptide and/or a protein component. In some cases, proteomic information may comprise primary structure information, secondary structure information, tertiary structure information, or quaternary information about the peptide or a protein. In some cases, proteomic information may comprise information about protein-ligand interactions, wherein a ligand may comprise any one of various biological molecules and substances that may be found in living organisms, such as, nucleotides, nucleic acids, amino acids, peptides, proteins, monosaccharides, polysaccharides, lipids, phospholipids, hormones, or any combination thereof.

In some cases, proteomic information may comprise information about a single cell, a tissue, an organ, a system of tissues and/or organs (such as cardiovascular, respiratory, digestive, or nervous systems), or an entire multicellular organism. In some cases, proteomic information may comprise information about an individual (e.g., an individual human being or an individual bacterium), or a population of individuals (e.g., human beings with diagnosed with cancer or a colony of bacteria). Proteomic information may comprise information from various forms of life, including forms of life from the Archaea, the Bacteria, the Eukarya, the Protozoa, the Chromista, the Plantae, the Fungi, or from the Animalia. In some cases, proteomic information may comprise information from viruses.

In some cases, proteomic information may comprise information relating exons and/or introns. In some cases, proteomic information may comprise information regarding variations in the primary structure, variations in the secondary structure, variations in the tertiary structure, or variations in the quaternary structure of peptides and/or proteins. In some cases, proteomic information may comprise information regarding variations in the expression of exons, including alternative splicing variations, structural variations, or both. In some cases, proteomic information may comprise conformation information, post-translational modification information, chemical modification information (e.g., phosphorylation), cofactor (e.g., salts or other regulatory chemicals) association information, or substrate association information of peptides and/or proteins.

In some cases, proteomic information may comprise information related to various proteoforms in a sample. In some cases, a proteomic information may comprise information related to peptide variants, protein variants, or both. In some cases, a proteomic information may comprise information related to splicing variants, allelic variants, post-translation modification variants, or any combination thereof. In some cases, peptide variants or protein variants may comprise a post-translation modification. In some cases, the post-translational modification comprises acylation, alkylation, prenylation, flavination, amination, deamination, carboxylation, decarboxylation, nitrosylation, halogenation, sulfurylation, glutathionylation, oxidation, oxygenation, reduction, ubiquitination, SUMOylation, neddylation, myristoylation, palmitoylation, isoprenylation, farnesylation, geranylgeranylation, glypiation, glycosylphosphatidylinositol anchor formation, lipoylation, heme functionalization, phosphorylation, phosphopantetheinylation, retinylidene Schiff base formation, diphthamide formation, ethanolamine phosphoglycerol functionalization, hypusine formation, beta-Lysine addition, acetylation, formylation, methylation, amidation, amide bond formation, butyrylation, gamma-carboxylation, glycosylation, polysialylation, malonylation, hydroxylation, iodination, nucleotide addition, phosphate ester formation, phosphoramidate formation, adenylation, uridylylation, propionylation, pyroglutamate formation, gluthathionylation, sulfenylation, sulfinylation, sulfonylation, succinylation, sulfation, glycation, carbonylation, isopeptide bond formation, biotinylation, carbamylation, oxidation, pegylation, citrullination, deamidation, eliminylation, disulfide bond formation, proteolytic cleavage, isoaspartate formation, racemization, protein splicing, chaperon-assisted folding, or any combination thereof.

### Composition Improving Assay

In some cases, a method of the present disclosure may comprise using a composition improving assay. In some cases, an untargeted assay may be a composition improving assay. In some cases, a composition improving assay may improve access to a subset of biomolecules in a biological sample. In some cases, a composition improving assay may improve detection to a subset of biomolecules in a biological sample. In some cases, a composition improving assay may improve identification to a subset of biomolecules in a biological sample. In some cases, the subset of biomolecules may be low-abundance biomolecules. In some cases, the subset of biomolecules may be rare biomolecules. In some cases, a rare biomolecule may be a biomolecule that is infrequently expressed by a cell. In some cases, a dynamic range of a biological sample may be compressed using a composition improving assay. In some cases, a dynamic range may be compressed by at least about 1, 2, 3, 4, 5, 6, 7, 8, 9, 10, 11, 12, 13, 14, or 15 orders of magnitude

In some cases, the composition improving assay may comprise providing one or more of surface regions comprising one or more surface types. In some cases, the composition improving assay may comprise contacting the biological sample with the one or more surface regions to yield a set of adsorbed biomolecules on the one or more surface regions. In some cases, the set of adsorbed biomolecules may comprise a reduced or compressed dynamic range compared to the same biomolecules in the biological sample that are not adsorbed. In some cases, the concentration of low abundance biomolecules in the biological sample may be increased on the one or more surface regions. In some cases, the concentration of high abundance biomolecules in the biological sample may be reduced on the one or more surface regions. In some cases, the composition improving assay may comprise desorbing, from the one or more surface regions, at least a portion of the set of adsorbed biomolecules to yield the set of polyamino acids. In some cases, the composition improving assay may comprise contacting the biological sample with the one or more surface regions to capture a set of biomolecules on the one or more surface regions. In some cases, the composition improving assay may comprise releasing, from the one or more surface regions, at least a portion of the set of biomolecules to yield the set of polyamino acids. In some cases, the one or more surface regions are disposed on a single continuous surface. In some cases, the one or more surface regions are disposed on one or more discrete surfaces. In some cases, the one or more discrete surfaces are surfaces of one or more particles. In some cases, the one or more particles may comprise a nanoparticle. In some cases, the one or more particles may comprise a microparticle. In some cases, the one or more particles may comprise a porous particle. In some cases, the one or more particles may comprise a bifunctional, trifunctional, or N-functional particle.

In some cases, the composition improving assay may comprise providing a plurality of surface regions comprising a plurality of surface types. In some cases, the composition improving assay may comprise contacting the biological sample with the plurality of surface regions to yield a set of adsorbed biomolecules on the plurality of surface regions. In some cases, the composition improving assay may comprise desorbing, from the plurality of surface regions, at least a portion of the set of adsorbed biomolecules to yield the set of polyamino acids. In some cases, the composition improving assay may comprise contacting the biological sample with the plurality of surface regions to capture a set of biomolecules on the plurality of surface regions. In some cases, the composition improving assay may comprise releasing, from the plurality of surface regions, at least a portion of the set of biomolecules to yield the set of polyamino acids. In some cases, the plurality of surface regions are disposed on a single continuous surface. In some cases, the plurality of surface regions are disposed on a plurality of discrete surfaces. In some cases, the plurality of discrete surfaces are surfaces of a plurality of particles. In some cases, the plurality of particles may comprise a nanoparticle. In some cases, the plurality of particles may comprise a microparticle. In some cases, the plurality of particles may comprise a porous particle. In some cases, the plurality of particles may comprise a bifunctional, trifunctional, or N-functional particle.

### Non-Specific Binding

A surface may bind biomolecules through variably selective adsorption (e.g., adsorption of biomolecules or biomolecule groups upon contacting the particle to a biological sample comprising the biomolecules or biomolecule groups, which adsorption is variably selective depending upon factors including e.g., physicochemical properties of the particle) or non-specific binding. Non-specific binding can refer to a class of binding interactions that exclude specific binding. Examples of specific binding may comprise protein-ligand binding interactions, antigen-antibody binding interactions, nucleic acid hybridizations, or a binding interaction between a template molecule and a target molecule wherein the template molecule provides a sequence or a 3D structure that favors the binding of a target molecule that comprise a complementary sequence or a complementary 3D structure, and disfavors the binding of a non-target molecule(s) that does not comprise the complementary sequence or the complementary 3D structure.

Non-specific binding may comprise one or a combination of a wide variety of chemical and physical interactions and effects. Non-specific binding may comprise electromagnetic forces, such as electrostatics interactions, London dispersion, Van der Waals interactions, or dipole-dipole interactions (e.g., between both permanent dipoles and induced dipoles). Non-specific binding may be mediated through covalent bonds, such as disulfide bridges. Non-specific binding may be mediated through hydrogen bonds. Non-specific binding may comprise solvophobic effects (e.g., hydrophobic effect), wherein one object is repelled by a solvent environment and is forced to the boundaries of the solvent, such as the surface of another object. Non-specific binding may comprise entropic effects, such as in depletion forces, or raising of the thermal energy above a critical solution temperature (e.g., a lower critical solution temperature). Non-specific binding may comprise kinetic effects, wherein one binding molecule may have faster binding kinetics than another binding molecule.

Non-specific binding may comprise a plurality of non-specific binding affinities for a plurality of targets (e.g., at least 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10,000, 20,000, 30,000, 40,000, 50,000 different targets adsorbed to a single particle, or at most 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10,000, 20,000, 30,000, 40,000, 50,000 different targets adsorbed to a single particle). The plurality of targets may have similar non-specific binding affinities that are within about one, two, or three magnitudes (e.g., as measured by non-specific binding free energy, equilibrium constants, competitive adsorption, etc.). This may be contrasted with specific binding, which may comprise a higher binding affinity for a given target molecule than non-target molecules.

Biomolecules may adsorb onto a surface through non-specific binding on a surface at various densities. In some cases, biomolecules or proteins may adsorb at a density of at least about 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 200, 300, 400, 500, 600, 700, 800, 900, or 1000 fg/mm². In some cases, biomolecules or proteins may adsorb at a density of at least about 1, 2, 3, 4, 5, 6, 7, 8, 9, 10, 20, 30, 40, 50, 60, 70, 80, 90, 100, 200, 300, 400, 500, 600, 700, 800, 900, or 1000 pg/mm². In some cases, biomolecules or proteins may adsorb at a density of at least about 1, 2, 3, 4, 5, 6, 7, 8, 9, 10, 20, 30, 40, 50, 60, 70, 80, 90, 100, 200, 300, 400, 500, 600, 700, 800, 900, or 1000 ng/mm². In some cases, biomolecules or proteins may adsorb at a density of at least about 1, 2, 3, 4, 5, 6, 7, 8, 9, 10, 20, 30, 40, 50, 60, 70, 80, 90, 100, 200, 300, 400, 500, 600, 700, 800, 900, or 1000 μg/mm². In some cases, biomolecules or proteins may adsorb at a density of at least about 1, 2, 3, 4, 5, 6, 7, 8, 9, 10, 20, 30, 40, 50, 60, 70, 80, 90, 100, 200, 300, 400, 500, 600, 700, 800, 900, or 1000 mg/mm². In some cases, biomolecules or proteins may adsorb at a density of at most about 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 200, 300, 400, 500, 600, 700, 800, 900, or 1000 fg/mm². In some cases, biomolecules or proteins may adsorb at a density of at most about 1, 2, 3, 4, 5, 6, 7, 8, 9, 10, 20, 30, 40, 50, 60, 70, 80, 90, 100, 200, 300, 400, 500, 600, 700, 800, 900, or 1000 pg/mm². In some cases, biomolecules or proteins may adsorb at a density of at most about 1, 2, 3, 4, 5, 6, 7, 8, 9, 10, 20, 30, 40, 50, 60, 70, 80, 90, 100, 200, 300, 400, 500, 600, 700, 800, 900, or 1000 ng/mm². In some cases, biomolecules or proteins may adsorb at a density of at most about 1, 2, 3, 4, 5, 6, 7, 8, 9, 10, 20, 30, 40, 50, 60, 70, 80, 90, 100, 200, 300, 400, 500, 600, 700, 800, 900, or 1000 μg/mm². In some cases, biomolecules or proteins may adsorb at a density of at most about 1, 2, 3, 4, 5, 6, 7, 8, 9, 10, 20, 30, 40, 50, 60, 70, 80, 90, 100, 200, 300, 400, 500, 600, 700, 800, 900, or 1000 mg/mm².

Adsorbed biomolecules may comprise various types of proteins. In some cases, adsorbed proteins may comprise at least about 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 types of proteins. In some cases, adsorbed proteins may comprise at most about 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 types of proteins.

In some cases, proteins in a biological sample may comprise at least about 1, 2, 3, 4, 5, 6, 7, 8, 9, 10, 15, 20, 25, or 30 orders of magnitudes in concentration. In some cases, proteins in a biological sample may comprise at most about 1, 2, 3, 4, 5, 6, 7, 8, 9, 10, 15, 20, 25, or 30 orders of magnitudes in concentration.

### Computer Systems

The present disclosure provides computer systems that are programmed to implement methods of the disclosure. FIG. 25 shows a computer system 2501 that is programmed or otherwise configured to, for example, culture a cell, transport biological samples, perform an assay for biomolecules, run analytical instruments, capture images of a cell culture, or any combination thereof.

The computer system 2501 may regulate various aspects of analysis, calculation, and generation of the present disclosure, such as, for example, for culturing a cell, transporting biological samples, performing an assay for biomolecules, running analytical instruments, capturing images of a cell culture, or any combination thereof. The computer system 2501 may be an electronic device of a user or a computer system that is remotely located with respect to the electronic device. The electronic device may be a mobile electronic device.

The computer system 2501 includes a central processing unit (CPU, also “processor” and “computer processor” herein) 2505, which may be a single core or multi core processor, or a plurality of processors for parallel processing. The computer system 2501 also includes memory or memory location 2510 (e.g., random-access memory, read-only memory, flash memory), electronic storage unit 2515 (e.g., hard disk), communication interface 2520 (e.g., network adapter) for communicating with one or more other systems, and peripheral devices 2525, such as cache, other memory, data storage and/or electronic display adapters. The memory 2510, storage unit 2515, interface 2520 and peripheral devices 2525 are in communication with the CPU 2505 through a communication bus (solid lines), such as a motherboard. The storage unit 2515 may be a data storage unit (or data repository) for storing data. The computer system 2501 may be operatively coupled to a computer network (“network”) 2530 with the aid of the communication interface 2520. The network 2530 may be the Internet, an internet and/or extranet, or an intranet and/or extranet that is in communication with the Internet.

The network 2530 in some cases is a telecommunication and/or data network. The network 2530 may include one or more computer servers, which may enable distributed computing, such as cloud computing. For example, one or more computer servers may enable cloud computing over the network 2530 (“the cloud”) to perform various aspects of analysis, calculation, and generation of the present disclosure, such as, for example, culturing a cell, transporting biological samples, performing an assay for biomolecules, running analytical instruments, capturing images of a cell culture, or any combination thereof. Such cloud computing may be provided by cloud computing platforms such as, for example, Amazon Web Services (AWS), Microsoft Azure, Google Cloud Platform, and IBM cloud. The network 2530, in some cases with the aid of the computer system 2501, may implement a peer-to-peer network, which may enable devices coupled to the computer system 2501 to behave as a client or a server.

The CPU 2505 may comprise one or more computer processors and/or one or more graphics processing units (GPUS). The CPU 2505 may execute a sequence of machine-readable instructions, which may be embodied in a program or software. The instructions may be stored in a memory location, such as the memory 2510. The instructions may be directed to the CPU 2505, which may subsequently program or otherwise configure the CPU 2505 to implement methods of the present disclosure. Examples of operations performed by the CPU 2505 may include fetch, decode, execute, and writeback.

The CPU 2505 may be part of a circuit, such as an integrated circuit. One or more other components of the system 2501 may be included in the circuit. In some cases, the circuit is an application specific integrated circuit (ASIC).

The storage unit 2515 may store files, such as drivers, libraries and saved programs. The storage unit 2515 may store user data, e.g., user preferences and user programs. The computer system 2501 in some cases may include one or more additional data storage units that are external to the computer system 2501, such as located on a remote server that is in communication with the computer system 2501 through an intranet or the Internet.

The computer system 2501 may communicate with one or more remote computer systems through the network 2530. For instance, the computer system 2501 may communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user may access the computer system 2501 via the network 2530.

The computer system 2501 may communicate with an apparatus as described herein. The apparatus may comprise one or more sensors, illustrated in FIG. 25 as an optical sensor 2560.

The apparatus and/or optical sensor 2560 may communicate with the computer system 2501 through network 2530.

Methods as described herein may be implemented by way of machine (e.g., computer processor) executable code stored on an electronic storage location of the computer system 2501, such as, for example, on the memory 2510 or electronic storage unit 2515. The machine executable or machine readable code may be provided in the form of software. During use, the code may be executed by the processor 2505. In some cases, the code may be retrieved from the storage unit 2515 and stored on the memory 2510 for ready access by the processor 2505. In some situations, the electronic storage unit 2515 may be precluded, and machine-executable instructions are stored on memory 2510.

The code may be pre-compiled and configured for use with a machine having a processer adapted to execute the code, or may be compiled during runtime. The code may be supplied in a programming language that may be selected to enable the code to execute in a pre-compiled or as-compiled fashion.

Aspects of the systems and methods provided herein, such as the computer system 2501, may be embodied in programming. Various aspects of the technology may be thought of as “products” or “articles of manufacture” typically in the form of machine (or processor) executable code and/or associated data that is carried on or embodied in a type of machine readable medium. Machine-executable code may be stored on an electronic storage unit, such as memory (e.g., read-only memory, random-access memory, flash memory) or a hard disk. “Storage” type media may include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 2501 may include or be in communication with an electronic display 2535 that comprises a user interface (UI) 2540 for, for example, culturing a cell, transporting biological samples, performing an assay for biomolecules, running analytical instruments, capturing images of a cell culture, or any combination thereof. Examples of UIs include, without limitation, a graphical user interface (GUI) and web-based user interface.

The computer system 2501 may further include additional peripheral devices

Methods and systems of the present disclosure may be implemented by way of one or more algorithms. An algorithm may be implemented by way of software upon execution by the central processing unit 2505. The algorithm can, for example, culture a cell, transport biological samples, perform an assay for biomolecules, run analytical instruments, capture images of a cell culture, of any combination thereof.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the present disclosure may be employed in practicing the present disclosure. It is intended that the following claims define the scope of the present disclosure and that methods and structures within the scope of these claims and their equivalents be covered thereby.

### EXAMPLES

The following examples are provided to further illustrate some embodiments of the present disclosure, but are not intended to limit the scope of the disclosure; it will be understood by their exemplary nature that other procedures, methodologies, or techniques known to those skilled in the art may alternatively be used.

### Example 1: Comparison of Mass Spectrometry Analysis Performed on Improved Secretome Compositions Versus Direct Digest

This example describes experiments conducted to study analytical improvements of performing mass spectrometry on secretome composition that was improved using particles versus direct digest secretome composition.

Hela S3 cells were cultured with F-12K medium supplemented with 10% fetal bovine serum and 1% PS solution. Cells were maintained at 37° C. with 95% air and 5% CO₂. Cell medium was removed from cell culture well by aspiration. The harvested medium was spun down before being analyzed on the Proteograph™ system.

Mass spectrometry was performed using Orbitrap-2 (with water column) on (a) secretome composition improved using particles (Proteograph™), (b) direct digest secretome, (c) and a control sample of HeLa digest. For (a), particles used were NP-1, NP-2, NP-3, NP-4, and NP-5.

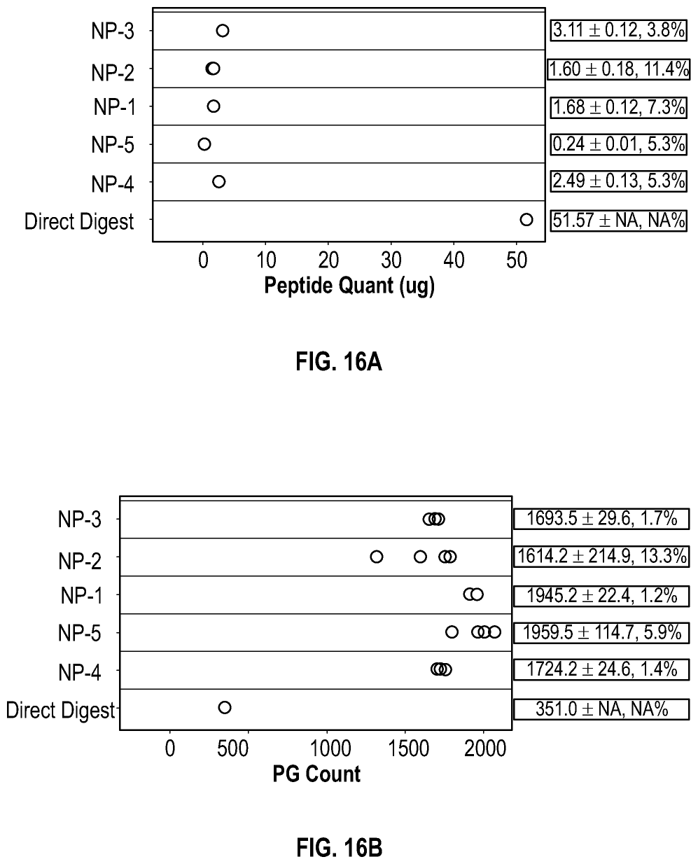

FIGS. 16A-16B show the mass of peptides and the number of protein groups identified from the experiments, respectively. The mass of peptides obtained from the direct digest (about 51.7 μg) far exceeded the mass obtained from the secretome composition using particles (about 3.11, 1.60, 1.68, 0.24, and 2.49 μg for NP-3, NP-2, NP-1, NP-5, and NP-4, respectively). However, the number of protein groups identified using the improved secretome composition (about 1694, 1614, 1945, 1959, and 1724 groups for NP-3, NP-2, NP-1, NP-5, and NP-4, respectively) was far greater than the number obtained from the direct digest (about 351 groups). These results are consistent with the particles enriching for less abundant proteins in the secretome composition.

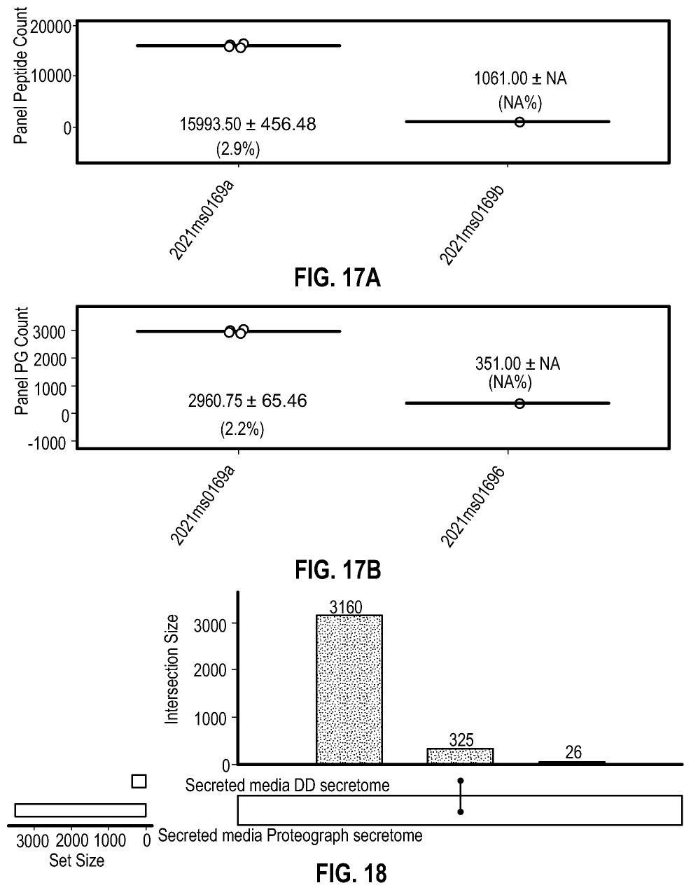

FIGS. 17A-17B show the number of peptides and the number of protein groups identified, respectively, from the mass spectrometry experiments. The number of peptides and the number of protein groups identified using an improved secretome composition was far greater (about 15,994 peptides and 2961 protein groups) than the number identified from direct digest (about 1061 peptides and 351 protein groups). The results show that using the Proteograph™ to improve the composition of the secretome before mass spectrometry can offer significantly higher protein and/or peptide coverage than performing mass spectrometry on direct digest.

FIG. 18 shows the number of protein groups that were exclusively identified in an improved secretome composition (3160) or a direct digest (26), as well as the number of protein groups that were identified in both experiments (325). The results show that using the Proteograph™ to improve the composition of the secretome before mass spectrometry can detect almost all proteins that are detected in direct digest.

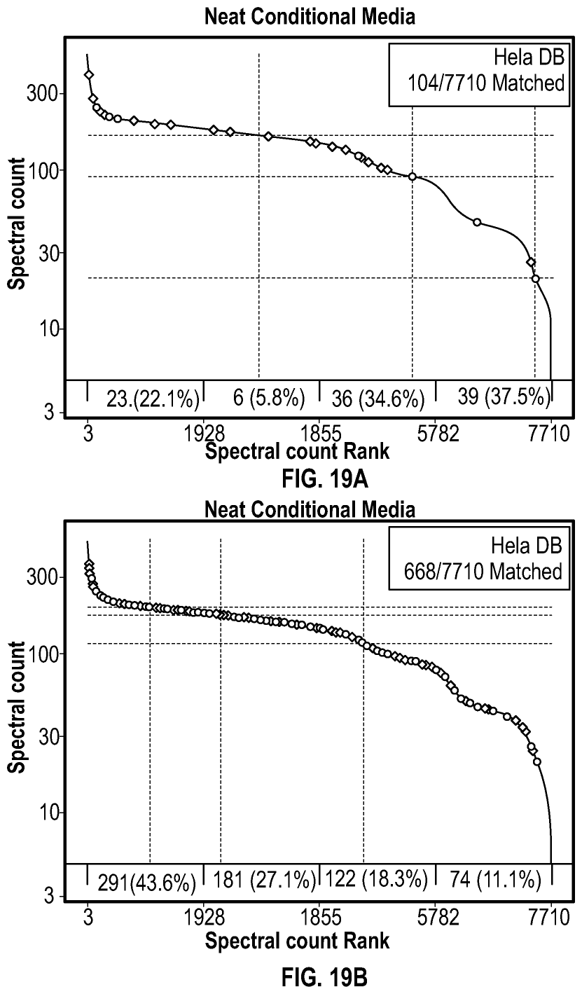

FIGS. 19A-19B show spectral count (representing protein abundance level; higher spectral counts indicate higher protein abundance) versus count rank of protein groups identified in the direct digest (neat conditional media) and improved secrotome (Protegraph Assay), respectively. Each spot in the plot represent a protein being detected at certain abundance level based on Hela DB database. Close to 10× more proteins were observed with Proteograph™ compared to neat medium alone, and proteins were observed across the entire concentration range of the HeLa proteome database, spanning at least 7 orders of magnitude. The identified protein groups are mapped onto the line in FIG. 19A and FIG. 19B. These figures further illustrate that mass spectrometry performed with the improved secretome composition leads to broad and deep coverage of the proteins in the secretome. It is noted that low-abundance cytokines and other low-abundance proteins were identified in the improved secretome composition.

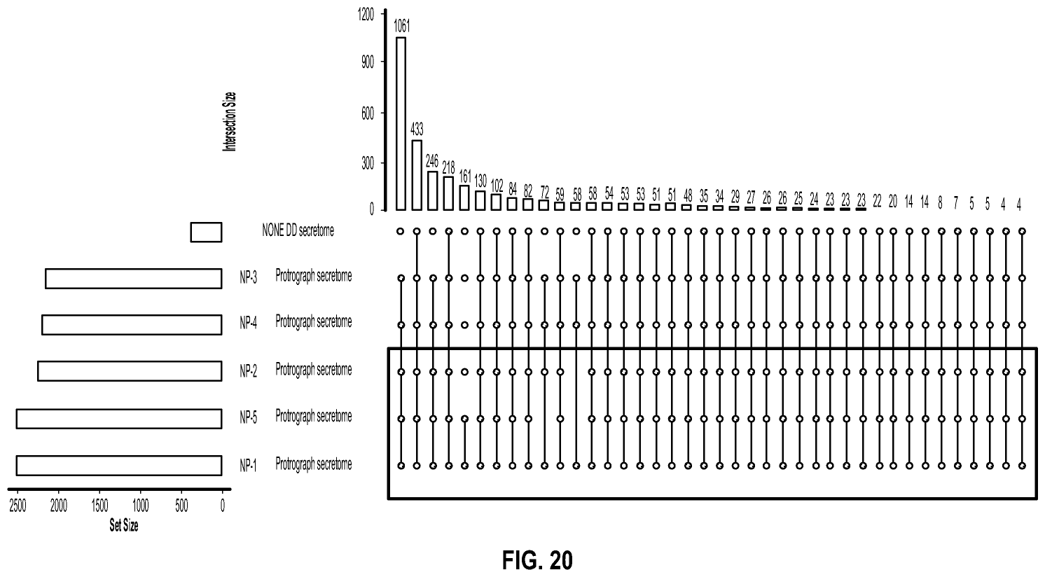

FIG. 20 shows the number of protein groups that were exclusively identified or mutually identified for the particles tested and the direct digest. The results show that, in some cases, a fewer number of particles (e.g., 2 or 3) can cover a large majority of the proteins and/or protein groups in the secretome. For example, the combination of NP-2, NP-5, and NP-1 (box in FIG. 20) covers 92% of protein groups (or 2737 groups) that were identified in the combination of all five particles tested.

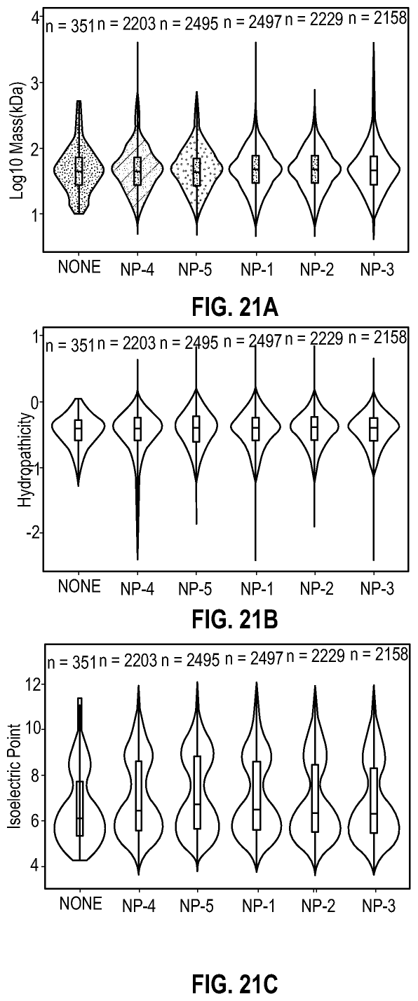

FIGS. 21A-21C show violin plots of mass distribution (FIG. 21A), hydropathicity distribution (FIG. 21B), and isoelectric point distribution (FIG. 21C) of the protein groups identified in the direct digest and the improved secretome composition, respectively. The results show that the distribution of protein properties for the proteins directed in the direct digest or the improved composition are similar, which suggests that improving the secretome composition with the Proteograph™ does not introduce significant bias in the proteins that are detected.

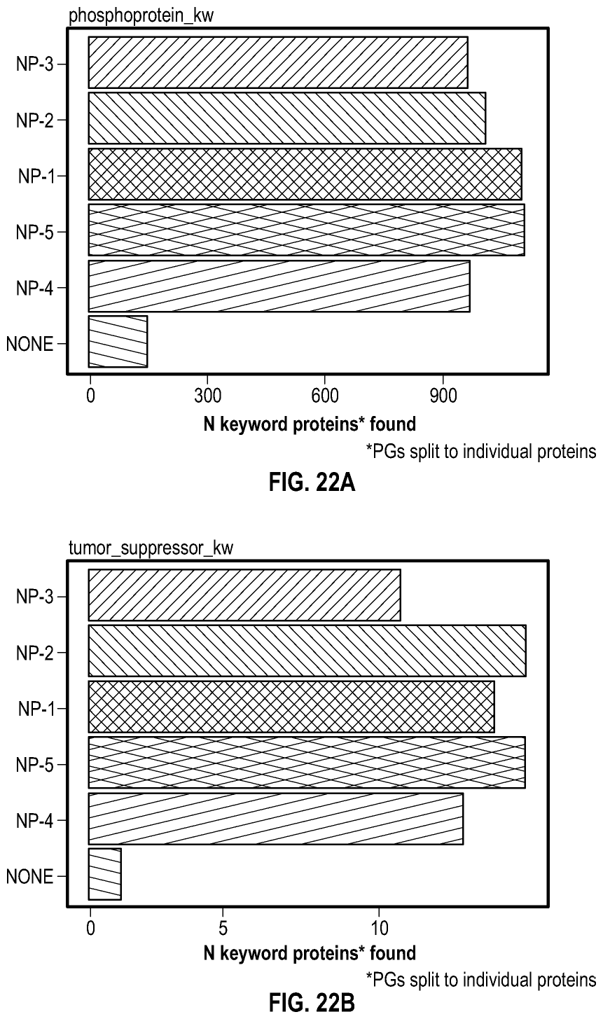

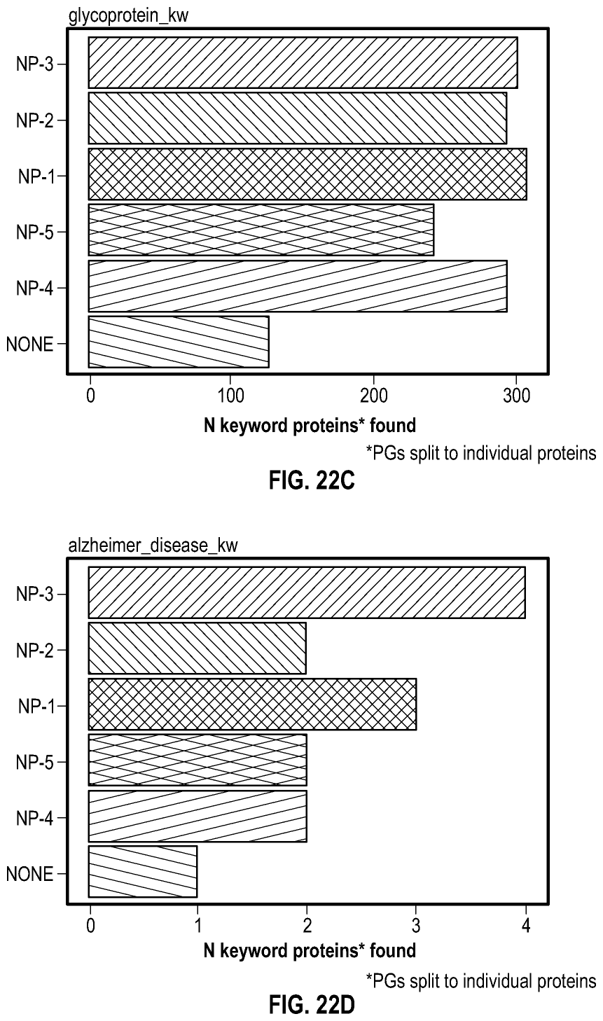

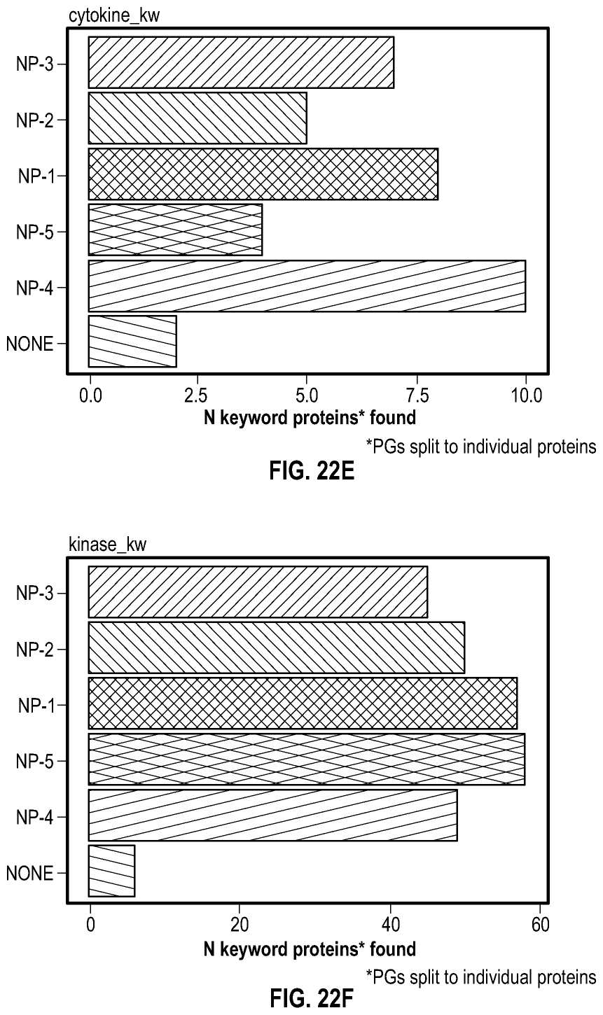

FIGS. 22A-22F show the number of proteins identified in the improved secretome composition (NP-1-NP-5) and the direct digest (“None”) for various protein keywords. Protein keywords considered were Phosphoproteins (FIG. 22A), Tumor Suppressors (FIG. 22B), Glycoproteins (FIG. 22C), Alzheimer's Disease (FIG. 22D), Cytokines (FIG. 22E), and Kinases (FIG. 22F). As illustrated in FIGS. 22A-22F, for each particle tested in the improved secretome composition, at least a few times more proteins were identified with the respective particle than from the direct digest, suggesting that improving the secretome composition as described herein can lead to the identification more relevant protein biomarkers associated with human disease than direct digest.

### Example 2: Evaluation of Engineered Multi-Nanoparticle-Based Proteomics Analysis for Unbiased, Deep, and Rapid Analysis of Fetal Bovine Serum Derived Cell Culture Media

In this study, the Proteograph™ workflow was first compared with conventional proteomic workflows for plasma proteomics analysis to demonstrate the advantage of Proteograph™ with its proprietary nanoparticles. Plasma samples were processed with one of four workflows: Proteograph™ workflow, high-pH fractionation, plasma depletion, and direct digestion of neat plasma. The study workflows are illustrated in FIG. 23A.

For secreted media, 250 μL of media containing 10% FBS was harvested from HeLa cells. The conditioned media were processed directly with the Proteograph™ automation instrument, and tryptic peptides were analyzed using a Thermo Fisher Scientific Orbitrap Fusion Lumos Tribrid Mass Spectrometer in a data-dependent acquisition (DDA) mode LC-MS analysis with a 2 h LC gradient per NP per sample. LC-MS/MS raw data files were processed using the Proteograph™ Analysis Suite with MaxQuant, applying 1% FDR cutoff at the protein and peptide levels.

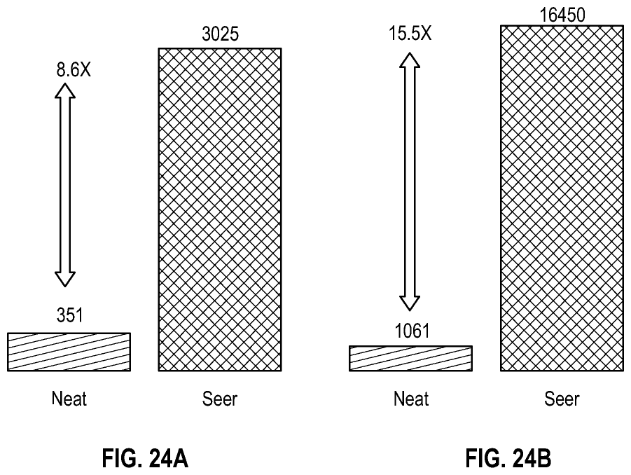

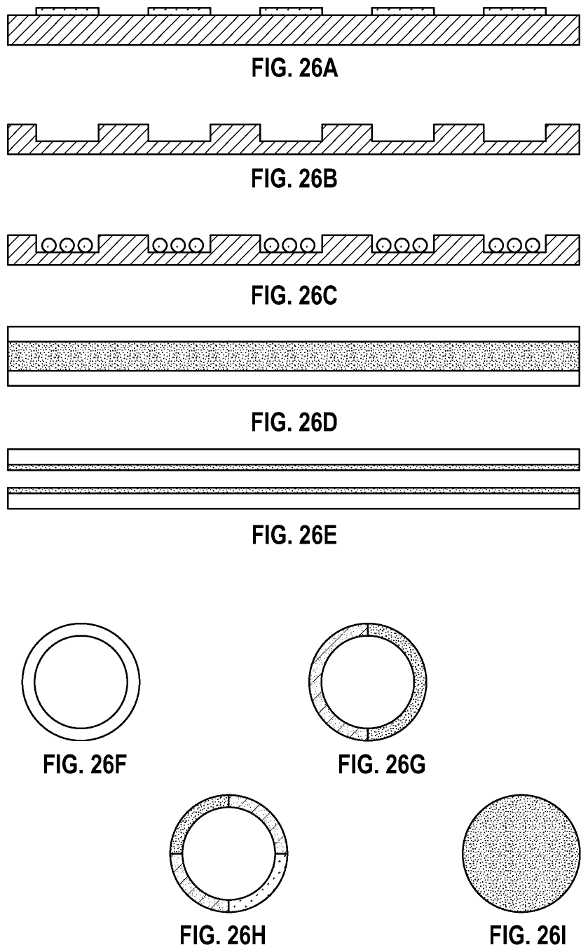

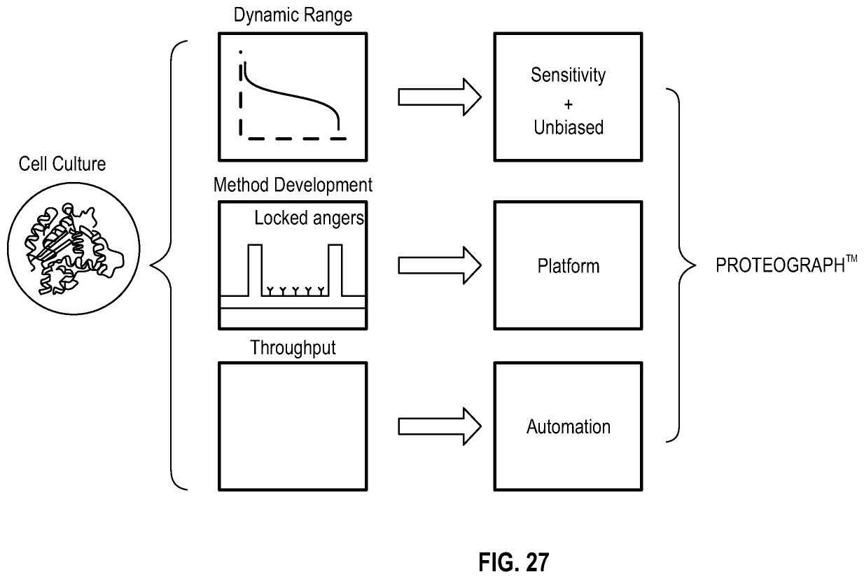

Other label-free proteomics workflows were compared to Proteograph™ Product Suite with a 120-minute DDA analysis for each five nanoparticles in the Proteograph™ Assay and total analysis time of 10 h per sample. The Proteograph™ data detected ˜3000 cell-derived protein groups (FIG. 23B and FIG. 24A) and over 16000 peptides (FIG. 24B) in the HeLa cell culture supernatant containing FBS (1% FDR at protein and peptide level) across 7 orders of magnitude dynamic range with MaxQuant. The Protegraph™ data allowed the identification of at least twice as many protein groups compared to high-pH fractionation and plasma depletion workflow (FIG. 23B) with improved replicate coefficients of variation as compared to high-pH fractionation (FIG. 23C).

The Proteograph™ platform offered about 10× or more improvement in depth of proteome coverage compared to results derived from direct digestion of same conditioned media material, enabling identification of lower abundance cytokines in the culture media, some of which were not robustly detected by some simple approaches or complicated workflows that involve depletion and fractionation. Identification of low abundant cytokines and other low abundance proteins was performed with Proteograph™ Product Suite. Table 1 lists examples of low abundance cytokines detected by Proteograph™

TABLE 1
Identification of Example Proteins in the Lower Abundance

| prime_protein_id | gene_names |
|---|---|
| Q99988 | GDF15 MIC1 PDF PLAB |
| Q14116 | IL18 IGIF IL1F4 |
| P43490 | NAMPT PBEF PBEF1 |
| P28799 | GRN |
| P22004 | BMP6 VGR |
| P19875 | CXCL2 GRO2 GROB |
| P13497 | BMP1 PCOLC |
| P14174 | MIF GLIF MMIF |
| O14793 | MSTN GDF8 |
| P12643 | BMP2 BMP2A |

The study shows that the performance of the Proteograph™ platform combined with label-free mass spectrometry analysis can enable deeper profiling of secreted proteins in cell culture media in a rapid fashion, and that the platform can enable deep and unbiased large-scale conditioned media studies to detect novel insights.

### Example 3: Evaluation of Engineered Multi-Nanoparticle-Based Proteomics Analysis for Unbiased, Deep, and Rapid Analysis of Host Cell Proteins in Drug Products

In this study, the Proteograph™ workflow was first compared with conventional proteomic workflows for plasma proteomics analysis to demonstrate the advantage of the Proteograph™ with its proprietary nanoparticles.

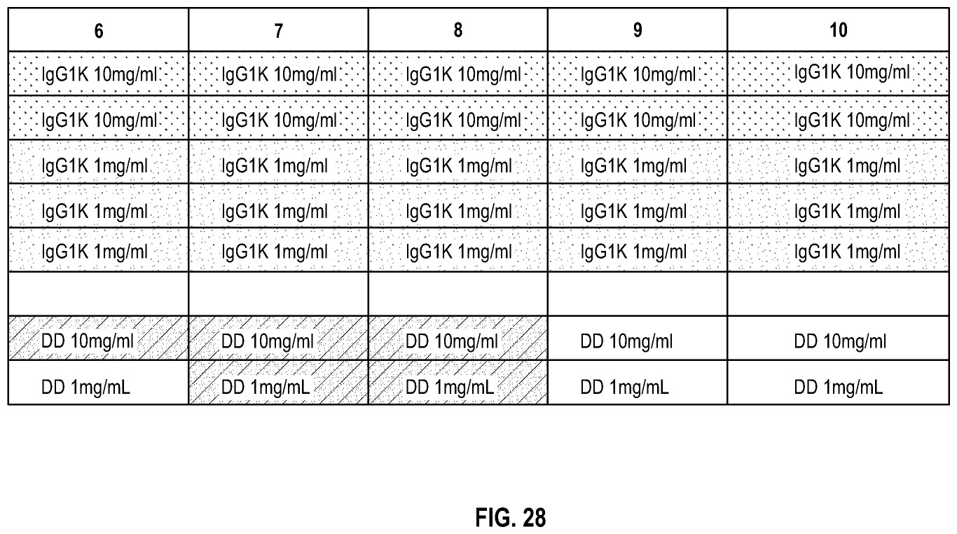

Samples from National Institute of Standards and Technology (NIST 8671, Humanized IgGIK mAb) were processed with the Proteograph™ workflow. One batch of samples was processed with the stock solution with the IgGIK mAb concentration at 10 mg/mL. Another batch of samples was processed after diluting the stock solution in PBS to achieve IgGIK mAb concentration at 1 mg/mL. 250 μL of the samples were used per Proteograph™ assay. For controls, a subset within each batch was processed without the Proteograph™ assay (“direct digest”, or DD). FIG. 28 shows an example plate layout for a single run of the Proteograph™ assay.

The LC-MS was configured with a Bruker timsTOF Pro2 using data independent acquisition (DIA). Total MS analysis time per injection was 33 minutes. In the experiments conducted with the Proteograph™, proteins were injected at 40, 100, or 200 ng of protein per μL depending on the experiment. For the experiments with the direct digest, proteins were injected at 200 ng of protein per μL.

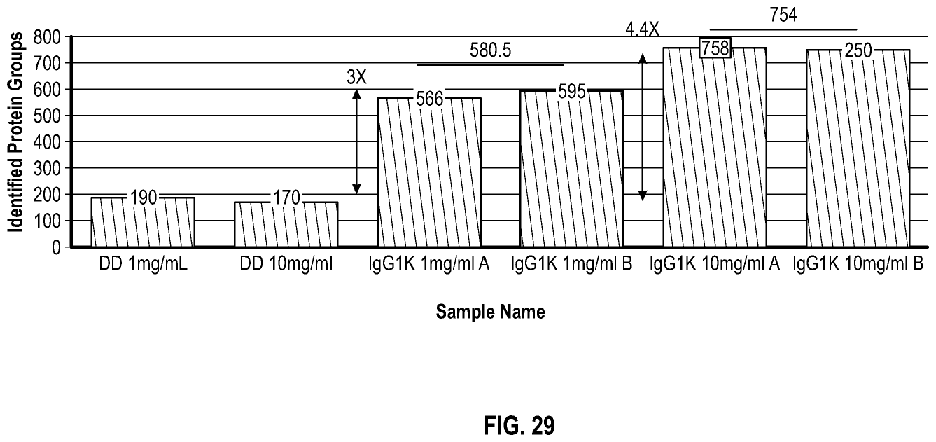

Data analysis was performed using the Proteograph Analysis Suite (PAS™ 1.5), using DIA-NN library-free search with a mouse reference proteome (UP000000589_10090). FIG. 29 shows the number of protein groups identified, in accordance with some embodiments. The number of protein groups identified with Proteograph™ was about 3 to about 5 times greater than number of protein groups identified with direct digest.

### LIST OF EMBODIMENTS

The following list of embodiments of the invention are to be considered as disclosing various features of the invention, which features can be considered to be specific to the particular embodiment under which they are discussed, or which are combinable with the various other features as listed in other embodiments. Thus, simply because a feature is discussed under one particular embodiment does not necessarily limit the use of that feature to that embodiment.

Embodiment 1. A method for selecting a candidate therapeutic for a disease, comprising: (a) incubating a cell in a predetermined environment comprising a therapeutic, such that the cell produces a biological sample comprising a plurality of biomolecules, wherein the biological sample is influenced at least in part by the therapeutic; (b) contacting the biological sample with a surface to adsorb the plurality of biomolecules onto the surface; (c) releasing at least a portion of the plurality of biomolecules on the surface; (d) detecting the at least the portion of the plurality of biomolecules, thereby identifying the plurality of biomolecules; and (e) selecting the therapeutic as a candidate therapeutic based at least partially on the plurality of biomolecules that are identified.

Embodiment 2. The method of embodiment 1, wherein the cell is afflicted with an infection or a mutation. Embodiment 3. The method of embodiment 1, wherein the cell is a cancer cell. Embodiment 4. The method of embodiment 3, wherein the cancer cell is a biopsied cell of a patient. Embodiment 5. The method of embodiment 1, wherein a particle comprises the surface. Embodiment 6. The method of embodiment 5, wherein the particle is a nanoparticle. Embodiment 7. The method of embodiment 1, wherein the contacting in (b) further comprises contacting the biological sample with a second surface to adsorb a second plurality of biomolecules onto the second surface. Embodiment 8. The method of embodiment 1, wherein the detecting comprises mass spectrometry. Embodiment 9. The method of embodiment 1, wherein the predetermined environment comprises a predetermined solvent environment, a predetermined temperature, or both. Embodiment 10. The method of embodiment 1, wherein the predetermined environment comprises a serum. Embodiment 11. The method of embodiment 1, wherein the biological sample comprises a secretome composition or an exosome. Embodiment 12. The method of embodiment 1, wherein (a) further comprising incubating a plurality of cells. Embodiment 13. The method of embodiment 12, wherein (a) further comprises incubating each cell in the plurality of cells in a plurality of predetermined environments. Embodiment 14. The method of embodiment 12, wherein each predetermined environment in the plurality of predetermined environments comprises a different therapeutic. Embodiment 15. The method of embodiment 12, wherein each cell in the plurality of cells comprises a different cell line. Embodiment 16. The method of embodiment 12, wherein each predetermined environment in the plurality of predetermined environments comprises a different predetermined solvent environment, a different predetermined temperature, or both. Embodiment 17. The method of embodiment 12, wherein each cell in the plurality of cells is incubated for a different time period.

Embodiment 18. A method for monitoring cell activity, comprising: (a) incubating a cell in a predetermined environment, such that the cell produces a biological sample comprising a plurality of biomolecules; (b) contacting the biological sample with a surface to adsorb the plurality of biomolecules onto the surface; (c) releasing at least a portion of the plurality of biomolecules on the surface; (d) detecting the at least the portion of the plurality of biomolecules, thereby identifying the plurality of biomolecules; and (e) repeating steps (a) through (d) after a predetermined amount of time, thereby monitoring the activity of the cell.

Embodiment 19. A method for identifying a biomolecule in a biological sample, comprising: (a) incubating a cell in a predetermined environment, such that the cell produces a biological sample; (b) contacting the biological sample with a surface to adsorb a plurality of low abundance biomolecules in the biological sample onto the surface; (c) releasing at least a portion of the plurality of low abundance biomolecules on the surface; and (d) detecting the at least the portion of the plurality of low abundance biomolecules, thereby identifying the plurality of low abundance biomolecules.

Embodiment 20. The method of embodiment 19, further comprising, before (c), adding a biomolecule to the biological sample, wherein the biomolecule reduces a detectability of the plurality of low abundance biomolecules in the biological sample.

Embodiment 21. An apparatus for assaying a biological sample, comprising: a substrate comprising a surface; a cell culture chamber comprising a cell; a loading unit that is operably coupled to the substrate and the cell culture chamber; and a computer readable medium comprising machine-executable code that, upon execution by a processor, implements a method comprising: providing a controlled environment to the cell in the cell culture chamber for a predetermined duration of time, such that the cell produces a biological sample in the cell culture chamber; transferring a portion of the biological sample from the cell culture chamber to the substrate using the loading unit, thereby contacting the portion with the surface to adsorb a biomolecule in the portion of the biological sample onto the surface; and assaying at least a portion of the biomolecule to detect the biomolecule in the biological sample.

Embodiment 22. A method for identifying one or more biomolecules, comprising: (a) providing a supplemented medium; (b) generating a set of biomolecules by incubating a cell in the supplemented medium under conditions sufficient for the cell to generate the set of biomolecules; (c) contacting at least a portion of the supplemented medium with one or more surfaces to adsorb the set of biomolecules; (d) removing the one or more surfaces and the set of biomolecules from the at least the portion of the supplemented medium to produce a separated sample; (e) releasing, in the separated sample, the set of biomolecules from the one or more surfaces; and (f) detecting at least a subset of the set of biomolecules, thereby identifying one or more biomolecules.

Embodiment 23. The method of embodiment 22, wherein, subsequent to the contacting of (b), the set of biomolecules comprises a reduced dynamic range when adsorbed on the one or more surfaces compared to an original dynamic range of the set of biomolecules in the supplemented medium. Embodiment 24. The method of embodiment 22 or 23, wherein, in (a), the incubating the cell in the supplemented medium is performed for less than about 5 seconds, 5 minutes, 5 hours, or 5 days. Embodiment 25. The method of any one of embodiments 22-24, wherein the cell is afflicted with an infection or a mutation. Embodiment 26. The method of any one of embodiments 22-25, wherein the cell is a viable cell comprising a cancer cell, an epithelial cell, a bone cell, a muscle cell, a fat cell, a tissue cell, a senescent cell, a pluripotent cell, a stem cell, or a nerve cell. Embodiment 27. The method of any one of embodiments 22-26, wherein the cell is a cancer cell that is a biopsied cell of a patient. Embodiment 28. The method of any one of embodiments 22-27, wherein a particle comprises the surface. Embodiment 29. The method of any one of embodiments 28, wherein the particle is a nanoparticle. Embodiment 30. The method of any one of embodiments 22-29, wherein the contacting in (b) further comprises contacting the biological sample with a second surface to adsorb a second plurality of biomolecules onto the second surface. Embodiment 31. The method of any one of embodiments 22-30, wherein the detecting comprises mass spectrometry. Embodiment 32. The method of any one of embodiments 22-31, wherein the conditions sufficient for the cell to generate the set of biomolecules with the supplemented medium comprises a predetermined temperature, a predetermined pressure, a predetermined flow regime, a predetermined solvent environment, or a combination thereof. Embodiment 33. The method of embodiment 32, wherein the conditions are kept constant. Embodiment 34. The method of any one of embodiments 22-33, wherein the supplemented medium comprises as a fraction or in whole a serum, a plasma, cerebral spinal fluid (CSF), synovial fluid (SF), urine, tears, crevicular fluid, semen, whole blood, milk, nipple aspirate, needle aspirate, ductal lavage, vaginal fluid, nasal fluid, ear fluid, gastric fluid, pancreatic fluid, trabecular fluid, lung lavage, prostatic fluid, sputum, fecal matter, bronchial lavage, fluid from swabbings, bronchial aspirants, sweat, saliva, or any combination thereof. Embodiment 35. The method of any one of embodiments 22-34, wherein the supplemented medium comprises a supernatant of a cell culture, a secretome of co-cultures, an exosome, a tissue or cell lysate, or any combination thereof. Embodiment 36. The method of any one of embodiments 22-35, wherein the supplemented medium comprises a synthetic supplemented medium. Embodiment 37. The method of any one of embodiments 22-36, wherein the set of biomolecules comprise one or more biomarkers, molecular signatures, secreted proteins, absorbed proteins, a secretome, an exosome, or any combination thereof. Embodiment 38. The method of any one of embodiments 1-37, further comprising repeating (a)-(e) for a second supplemented medium comprising a second set of biomolecules, wherein the second supplemented medium is generated by incubating the cell in the supplemented medium for a different length of time. Embodiment 39. The method of any one of embodiments 22-38, wherein the providing is performed by an automated fluidic system. Embodiment 40. The method of embodiment 39, wherein the automated fluidic system comprises a microfluidic system. Embodiment 41. The method of embodiment 39 or 40, wherein the automated fluidic system provides the supplemented medium at different times. Embodiment 42. The method of any one of embodiments 22-41, wherein the generating comprises an active secretion of at least a portion of the set of biomolecules. Embodiment 43. The method of any one of embodiments 22-42, wherein the generating comprises a passive release of at least a portion of the set of biomolecules. Embodiment 44. The method of any one of embodiments 22-43, wherein the generating comprises release of an exosome or liposome by the cell. Embodiment 45. The method of any one of embodiments 22-44, wherein the generating comprises apoptosis of the cell. Embodiment 46. The method of any one of embodiments 22-45, wherein the generating comprises necroptosis of the cell. Embodiment 47. The method of any one of embodiments 22-46, wherein a biomolecule of the set of biomolecules is a complex. Embodiment 48. The method of any one of embodiments 22-47, wherein a biomolecule of the set of biomolecules is a protein. Embodiment 49. The method of any one of embodiments 22-48, wherein a biomolecule of the set of biomolecules is a polypeptide. Embodiment 50. The method of any one of embodiments 22-49, wherein a biomolecule of the set of biomolecules is a nucleic acid. Embodiment 51. The method of any one of embodiments 22-50, wherein the cell is part of a plurality of cells. Embodiment 52. The method of embodiment 51, wherein the plurality of cells are cells of a same type. Embodiment 53. The method of any one of embodiments 22-52, wherein the cell is of a tissue sample, an organoid, an immortalized cell line, or any combination thereof. Embodiment 54. The method of any one of embodiments 22-53, wherein the cell is a stem cell. Embodiment 55. The method of any one of embodiments 22-54, wherein the conditions sufficient for the cell to exchange the set of biomolecules with the supplemented medium comprises one or more of a presence or absence of an organic compound, a presence or absence of an inorganic compound, a presence or absence of an autocrine signaling molecule, a presence or absence of a paracrine signaling molecule, a presence or absence of an antigen, a presence or absence of one or more co-cultured cells, a presence or absence of radiation, a presence or absence of one or more toxins, a presence or absence of protein aggregates, a presence or absence of one or more proteins, a presence or absence of active viral particles, a presence or absence of inactivated viral particles, a presence or absence of applied heat or cooling, a presence or absence of applied mechanical stress, a presence or absence of electrical stimulation, a presence or absence of transposons, a presence or absence of exosomes, a presence or absence of liposomes, a presence or absence of coated nucleic acids, a presence or absence of shock, or any combination thereof. Embodiment 56. The method of embodiment 55, wherein the conditions are varied over time. Embodiment 57. The method of any one of embodiments 22-56, wherein the contacting in (b) is performed such that the portion of the supplemented medium contacts one or more surface regions of the one or more surfaces, wherein the one or more surfaces regions do not comprise a specific targeting moiety. Embodiment 58. The method of any one of embodiments 1-57, further comprising determining one or more protein to protein interactions, biomarkers, molecular signatures, biomolecules absorbed by the cell, biomolecules secreted by the cell, biomolecules dissociating from cell surfaces, biomolecules being cleaved off or shed from the surface, macromolecular complexes budded, released of cleaved of the surface, biomolecules passively released by the cell, conventionally and unconventionally released proteins, apoptotic release of biomolecules, necrotic released biomolecules, post-translation modifications, cell to cell interactions, cell to cell communications, or any combination thereof based at least in part on the identification, wherein molecular signatures are any patterns of proteins/proteoforms indicative of a biological state. Embodiment 59. The method of embodiment 58, wherein the one or more biomolecules secreted by the cell are conventional secretions, unconventional secretions, type 1 unconventional secretions, or type 2 unconventional secretions. Embodiment 60. The method of any one of embodiments 22-59, wherein the set of biomolecules comprises a biomolecule assembly. Embodiment 61. The method of embodiment 60, wherein the biomolecule assembly comprises quaternary protein, a vesicle, or an exosome. Embodiment 62. The method of any one of embodiments 22-61, wherein the cell is a eukaryote or a prokaryote. Embodiment 63. The method of any one of embodiments 22-62, further comprising a plurality of cells comprising the cell. Embodiment 64. The method of embodiment 63, wherein the plurality of cells is comprised in a tissue, an organoid, an organism, or a plurality of organisms. Embodiment 65. The method of embodiment 63, wherein the plurality of cells comprises at least a first cell of a first type and a second cell of a second type, such that the first cell exchanges one or more biomolecules of the set of biomolecules with the second cell. Embodiment 66. The method of embodiment 65, wherein the first cell and the second cell are co-cultured. Embodiment 67. The method of embodiment 65 or 66, wherein the first cell is comprised in a feeder culture for the second cell. Embodiment 68. The method of any one of embodiments 22-67, wherein the cell is derived from an immortalized cell line. Embodiment 69. The method of embodiment 68, wherein the cell is a HeLa cell. Embodiment 70. The method of any one of embodiments 22-69, wherein the cell is comprised in a primary cell culture. Embodiment 71. The method of any one of embodiments 63-70, wherein the plurality of cells are disposed in a plurality of separate volumes, each volume comprising a different supplemented medium or incubating conditions. Embodiment 72. The method of any one of embodiments 22-71, wherein the releasing comprises use of a protease. Embodiment 73. The method of any one of embodiments 22-72, wherein the one or more surfaces comprise at least two surfaces comprising distinct physicochemical properties such that the at least two surfaces adsorb a different pattern of biomolecule abundance from the set of biomolecules. Embodiment 74. The method of any one of embodiments 1-73, further comprising determining the one or more biomolecules were generated by the cell and not originally present in the supplemented medium. Embodiment 75. The method of any one of embodiments 22-74, wherein the supplemented medium comprises fetal bovine serum.

Embodiment 76. A method for monitoring cell activity, comprising: (a) incubating a cell such that the cell generates a biological sample comprising a plurality of biomolecules; (b) contacting the biological sample with a surface to adsorb the plurality of biomolecules onto the surface; (c) separating the surface from the biological sample; (d) releasing at least a portion of the plurality of biomolecules on the surface; (e) detecting the at least the portion of the plurality of biomolecules, thereby identifying the plurality of biomolecules; and (f) repeating (a) through (d) after a predetermined amount of time, thereby monitoring an activity of the cell.

Embodiment 77. The method of embodiment 76, wherein the cell generates the biological sample by any one of producing, releasing, adsorbing, digesting, or modifying a biomolecule of the plurality of biomolecules. Embodiment 78. The method of embodiment 76 or 77, further comprising changing an incubation condition for the cell based at least in part on the activity of the cell. Embodiment 79. The method of any one of embodiments 76-78, wherein the incubating comprises exposing the cell to a supplemented medium, wherein the supplemented medium obscures detection of the at least the portion of the plurality of biomolecules. Embodiment 80. The method of any one of embodiments 76-79, further comprising analyzing the activity of the cell. Embodiment 81. The method of any one of embodiments 76-80, wherein the at least the portion of the plurality of biomolecules comprise a dynamic range of at least about 6, 7, 8, 9, 10, 11, 12, 13, 14, 15, 16, 17, 18, 19, or 20. Embodiment 82. The method of any one of embodiments 76-81, wherein the at least the portion of the plurality of biomolecules comprise at least about 500, 600, 700, 800, 900, 1000, 2000, 3000, 4000, 5000, 6000, 7000, 8000, 9000, 10000, 20000, 30000, 40000, or 50000 biomolecules. Embodiment 83. The method of any one of embodiments 76-82, wherein (b) comprises contacting the biological sample with a plurality of surfaces. Embodiment 84. The method of any one of embodiments 76-83, wherein the releasing in (d) is performed using a protease that enzymatically cleaves the surface. Embodiment 85. The method of any one of embodiments 76-84, wherein the surface is disposed on a magnetic substrate, and wherein the separating in (c) is performed using a magnetic field to separate the magnetic substrate from the biological sample.

Embodiment 86. A method for identifying a low abundance biomolecule in a biological sample, comprising: (a) incubating a cell in a predetermined environment, such that the cell produces a biological sample; (b) contacting the biological sample with a surface to adsorb a plurality of low abundance biomolecules in the biological sample onto the surface; (c) separating the surface from the biological sample; (d) releasing at least a portion of the plurality of low abundance biomolecules on the surface; and (e) detecting the at least the portion of the plurality of low abundance biomolecules, thereby identifying the plurality of low abundance biomolecules.

Embodiment 87. The method of embodiment 86, further comprising, before (c), adding a biomolecule to the biological sample, wherein the biomolecule reduces a detectability of the plurality of low abundance biomolecules in the biological sample. Embodiment 88. The method of embodiment 86 or 87, wherein the detecting a low abundance biomolecule is about 7 orders of magnitude higher in signal than a signal from direct digestion of the cell in media. Embodiment 89. The method of any one of embodiments 86-88, wherein the incubating comprises exposing the cell to a supplemented medium, wherein the supplemented medium obscures detection of the at least the portion of the plurality of low abundance biomolecules. Embodiment 90. The method of any one of embodiments 86-89, wherein the plurality of low abundance biomolecules comprises at least about 500, 600, 700, 800, 900, 1000, 2000, 3000, 4000, 5000, 6000, 7000, 8000, 9000, 10000, 20000, 30000, 40000, or 50000 biomolecules. Embodiment 91. The method of any one of embodiments 86-90, wherein (b) comprises contacting the biological sample with a plurality of surfaces. Embodiment 92. The method of any one of embodiments 86-91, wherein the releasing in (d) is performed using a protease that enzymatically cleaves the surface. Embodiment 93. The method of any one of embodiments 86-92, wherein the surface is disposed on a magnetic substrate, and wherein the separating in (c) is performed using a magnetic field to separate the magnetic substrate from the biological sample.

Embodiment 94. An apparatus for assaying a biological sample, comprising: a substrate comprising a surface; a cell culture chamber comprising a cell; a loading unit that is operably coupled to the substrate and the cell culture chamber; and a computer readable medium comprising machine-executable code that, upon execution by a processor, implements a method comprising: i. providing a controlled environment to the cell in the cell culture chamber for a predetermined duration of time, such that the cell produces a biological sample in the cell culture chamber; ii. transferring at least a portion of the biological sample from the cell culture chamber to the substrate using the loading unit, thereby contacting the at least the portion with the surface to adsorb a biomolecule in the at least the portion of the biological sample onto the surface; and iii. assaying at least a portion of the biomolecule to detect the biomolecule in the biological sample.

Embodiment 95. The apparatus of embodiment 94, comprising a plurality of cell culture chambers. Embodiment 96. The apparatus of embodiment 95, wherein a first cell culture chamber in the plurality of cell culture chambers is provided with a first controlled environment and a second cell culture chamber in the plurality of cell culture chambers is provided with a second controlled environment. Embodiment 97. The apparatus of any one of embodiments 94-96, wherein the transferring is performed using one or more fluidic connections and one or more pumps comprised within the loading unit. Embodiment 98. The apparatus of any one of embodiments 94-97, wherein the transferring is performed using one or more pipettes and one or more pumps comprised within the loading unit.

Embodiment 99. A method for identifying one or more biomolecules, comprising: (a) incubating a cell under conditions sufficient for the cell to generate an exosome, wherein the exosome comprises a plurality of biomolecules; (b) contacting the exosome with one or more surfaces to capture at least a portion of the exosome; (c) removing the one or more surfaces and the at least the portion of the exosome from the cell to produce a separated sample; (d) releasing, in the separated sample, the at least the portion of the exosome from the one or more surfaces; and (e) detecting at least a subset of the plurality of biomolecules in the at least the portion of the exosome, thereby identifying one or more biomolecules.

Embodiment 100. A method for monitoring cell activity, comprising: (a) incubating a cell such that the cell generates a biological sample comprising an exosome, wherein the exosome comprises a plurality of biomolecules; (b) contacting the biological sample with a surface to capture at least a portion of the exosome onto the surface; (c) releasing the at least the portion of the exosome from the surface; (d) detecting the at least the portion of the plurality of biomolecules in the at least the portion of the exosome, thereby identifying the plurality of biomolecules; and (e) repeating (a) through (d) after a predetermined amount of time, thereby monitoring an activity of the cell.

Embodiment 101. A method for identifying a low abundance biomolecule in a biological sample, comprising: (a) incubating a cell in a predetermined environment, such that the cell produces a biological sample comprising an exosome, wherein the exosome comprises a plurality of low-abundance biomolecules; (b) contacting the biological sample with a surface to capture at least a portion of the exosome in the biological sample onto the surface; (c) releasing the at least the portion of the exosome from the surface; and (d) detecting at least the portion of the plurality of low abundance biomolecules in the at least the portion of the exosome, thereby identifying the plurality of low abundance biomolecules.

Embodiment 102. An apparatus for assaying a biological sample, comprising: a substrate comprising a surface; a cell culture chamber comprising a cell; a loading unit that is operably coupled to the substrate and the cell culture chamber; and a computer readable medium comprising machine-executable code that, upon execution by a processor, implements a method comprising: i. providing a controlled environment to the cell in the cell culture chamber for a predetermined duration of time, such that the cell produces a biological sample comprising an exosome in the cell culture chamber, wherein the exosome comprises one or more biomolecules; ii. transferring at least a portion of the biological sample from the cell culture chamber to the substrate using the loading unit, thereby contacting the at least the portion with the surface to adsorb the exosome in the at least the portion of the biological sample onto the surface; and iii. assaying the exosome to detect the one or more biomolecules in the biological sample.

Embodiment 103. A method of identifying biomolecules, comprising: (a) processing one or more exosomes to release a plurality of biomolecules in the one or more exosomes to an environment external to the one or more exosomes, wherein a subset of biomolecules in the plurality of biomolecules comprises a first distribution of relative abundances in the one or more exosomes; (b) performing a composition improving assay on the plurality of biomolecules to increase the first distribution to a second distribution of relative abundances for the subset of biomolecules; (c) assaying the plurality of biomolecules to identify the subset of biomolecules.

Embodiment 104. The method of embodiment 103, wherein the processing is performed at least partially by adding a lyse buffer to the one or more exosomes. Embodiment 105. The method of embodiment 103 or 104, wherein the processing is performed at least partially by providing acoustic energy to the one or more exosomes. Embodiment 106. The method of any one of embodiments 103-105, wherein the processing is performed at least partially by freezing and thawing the one or more exosomes. Embodiment 107. The method of any one of embodiments 103-106, wherein the processing is performed at least partially by heating the one or more exosomes. Embodiment 108. The method of any one of embodiments 103-107, wherein the processing is performed at least partially by shearing the one or more exosomes. Embodiment 109. The method of any one of embodiments 103-108, wherein the processing is performed at least partially by grinding the one or more exosomes. Embodiment 110. The method of any one of embodiments 103-109, wherein the subset of biomolecules comprises at least about 100, 200, 300, 400, 500, 600, 700, 800, 900, 1,000, 2,000, 3,000, 4,000, 5,000, 6,000, 7,000, 8,000, 9,000, 10,000, 20,000, 30,000, 30,000, 40,000, 50,000, 60,000, 70,000, 80,000, 90,000, or 100,000 unique biomolecules. Embodiment 111. The method of any one of embodiments 103-110, wherein the composition improving assay comprises contacting the plurality of biomolecules with one or more surfaces to adsorb the subset of biomolecules on the one or more surfaces, such that the subset of biomolecules comprises the second distribution when the subset of biomolecules is adsorbed on the one or more surfaces. Embodiment 112. The method of any one of embodiments 103-111, wherein the contacting increases a visibility of the subset of biomolecules in the assaying step of (c) when the subset of biomolecules are adsorbed on the one or more surfaces, compared to the visibility of the subset of biomolecules when the subset of biomolecules are in the one or more exosomes, wherein the subset of biomolecules are low-abundance biomolecules comprising less than about 1 percent by mass of the plurality of biomolecules in the one or more exosomes.

Embodiment 113. A method of identifying biomolecules, comprising: (a) contacting a biological sample comprising one or more exosomes with a plurality of particles to non-specifically bind a subset of the one or more exosomes on the plurality of particles, wherein the plurality of particles comprises distinct physicochemical properties, and wherein the one or more exosomes comprise a plurality of biomolecules; (b) processing the one or more exosomes to release the plurality of biomolecules to an environment external to the one or more exosomes;

and (c) assaying the plurality of biomolecules to identify at least a subset of biomolecules in the plurality of biomolecules.

Embodiment 114. The method of embodiment 113, wherein the processing is performed at least partially by adding a lyse buffer to the one or more exosomes. Embodiment 115. The method of embodiment 113 or 114, wherein the processing is performed at least partially by providing acoustic energy to the one or more exosomes. Embodiment 116. The method of any one of embodiments 113-115, wherein the processing is performed at least partially by freezing and thawing the one or more exosomes. Embodiment 117. The method of any one of embodiments 113-116, wherein the processing is performed at least partially by heating the one or more exosomes. Embodiment 118. The method of any one of embodiments 113-117, wherein the processing is performed at least partially by shearing the one or more exosomes. Embodiment 119. The method of any one of embodiments 113-118, wherein the processing is performed at least partially by grinding the one or more exosomes. Embodiment 120. The method of any one of embodiments 113-119, wherein the subset of biomolecules comprises at least about 100, 200, 300, 400, 500, 600, 700, 800, 900, 1,000, 2,000, 3,000, 4,000, 5,000, 6,000, 7,000, 8,000, 9,000, 10,000, 20,000, 30,000, 30,000, 40,000, 50,000, 60,000, 70,000, 80,000, 90,000, or 100,000 unique biomolecules. Embodiment 121. The method of any one of embodiments 113-120, wherein the plurality of particles increases a visibility of the subset of biomolecules in the assaying step of (c) when the subset of biomolecules are adsorbed on the plurality of particles, compared to the visibility of the subset of biomolecules when the subset of biomolecules are in the one or more exosomes, wherein the subset of biomolecules are low-abundance biomolecules comprising less than about 1 percent by mass of the plurality of biomolecules in the one or more exosomes.

Embodiment 122. A method for characterizing a biological preparation, comprising: (a) contacting the biological preparation with one or more surfaces to adsorb a plurality of biomolecules in the biological preparation onto the one or more surfaces, wherein the plurality of biomolecules comprises a product biomolecule and an impurity; and (b) assaying the plurality of biomolecules to determine a deviation between a composition of the plurality of biomolecules and a reference composition, wherein the deviation is indicative of a purity or an activity of the biological preparation.

Embodiment 123. The method of embodiment 122, wherein the biological preparation comprises a cell culture. Embodiment 124. The method of embodiment 123, wherein the plurality of biomolecules comprises a secretome or an exosome from the cell culture.

Embodiment 125. The method of embodiment 123 or 124, wherein the plurality of biomolecules comprises a portion of one or more cells of the cell culture. Embodiment 126. The method of any one of embodiments 123-125, wherein the cell culture comprises a host organism that produces the product biomolecule. Embodiment 127. The method of any one of embodiments 123-126, wherein the cell culture comprises a host organism that produces the impurity. Embodiment 128. The method of any one of embodiments 123-127, wherein the cell culture comprises a contaminating organism that produces the impurity. Embodiment 129. The method of embodiment 127, wherein the impurity comprises a host-cell protein. Embodiment 130. The method of embodiment 129, wherein the host-cell protein comprises a protease, a lipase, or an isomerase. Embodiment 131. The method of embodiment 129 or 130, wherein the host-cell protein comprises a passively released protein or an actively released protein. Embodiment 132. The method of any one of embodiments 129-131, wherein the host-cell protein comprises a part of the host organism. Embodiment 133. The method of embodiment 132, wherein the part comprises a cell membrane, an organelle, or both Embodiment 134. The method of any one of embodiments 123-133, wherein the cell culture comprises a plurality of host organism species or strains. Embodiment 135. The method of any one of embodiments 123-134, wherein the cell culture comprises a plurality of contaminating species or strains. Embodiment 136. The method of any one of embodiments 123-135, wherein biological preparation comprises a plurality of impurities. Embodiment 137. The method of any one of embodiments 123-136, wherein biological preparation comprises a plurality of product biomolecules. Embodiment 138. The method of any one of embodiments 126-137, wherein the host organism comprises a prokaryotic host organism or a eukaryotic host organism. Embodiment 139. The method of embodiment 138, wherein the prokaryotic host organism comprises Escherichia coli, Streptomyces sp., acetic acid bacteria, lactic acid bacteria, a thermophilic Bacillus sp., Clostridium thermocellus, Agrobacterium tumefaciens, Thermus aquaticus, Bacillus coagulans, Pseudomonas stutzeri, Acetobacter sp., Micrococcus sp., Haemophilus influenzae, or Leuconostoc mesenteroides. Embodiment 140. The method of embodiment 138, wherein the eukaryotic host organism comprises a yeast, a fungus, a HeLa cell, a stem cell, a cancer cell, a genetically modified cell, or an algae. Embodiment 141. The method of embodiment 140, wherein the genetically modified cell comprises an exogenous nucleic acid sequence that encodes the product biomolecule. Embodiment 142. The method of any one of embodiments 122-141, wherein the impurity is a proteoform of a biomolecule in the plurality of biomolecules. Embodiment 143. The method of any one of embodiments 122-142, wherein the impurity is a proteoform of the product biomolecule. Embodiment 144. The method of embodiment 143, wherein the proteoform comprises a splicing variant, an allelic variant, or a post-translational modification variant. Embodiment 145. The method of embodiment 144, wherein the post-translational modification variant comprises a post-translational modification comprising: acylation, alkylation, prenylation, flavination, amination, deamination, carboxylation, decarboxylation, nitrosylation, halogenation, sulfurylation, glutathionylation, oxidation, oxygenation, reduction, ubiquitination, SUMOylation, neddylation, myristoylation, palmitoylation, isoprenylation, farnesylation, geranylgeranylation, glypiation, glycosylphosphatidylinositol anchor formation, lipoylation, heme functionalization, phosphorylation, phosphopantetheinylation, retinylidene Schiff base formation, diphthamide formation, ethanolamine phosphoglycerol functionalization, hypusine formation, beta-Lysine addition, acetylation, formylation, methylation, amidation, amide bond formation, butyrylation, gamma-carboxylation, glycosylation, polysialylation, malonylation, hydroxylation, iodination, nucleotide addition, phosphate ester formation, phosphoramidate formation, adenylation, uridylylation, propionylation, pyroglutamate formation, gluthathionylation, sulfenylation, sulfinylation, sulfonylation, succinylation, sulfation, glycation, carbonylation, isopeptide bond formation, biotinylation, carbamylation, oxidation, pegylation, citrullination, deamidation, eliminylation, disulfide bond formation, proteolytic cleavage, isoaspartate formation, racemization, protein splicing, chaperon-assisted folding, or any combination thereof. Embodiment 146. The method of any one of embodiments 122-145, wherein a difference between a first log (water-octanol partition coefficient) the impurity and a second log (water-octanol partition coefficient) the product biomolecule is less than about 2, 1.5, 1, 0.9, 0.8, 0.7, 0.6, 0.5, 0.4, 0.3, 0.2, 0.1, 0.09, 0.08, 0.07, 0.06, 0.05, 0.04, 0.03, 0.02, or 0.01. Embodiment 147. The method of any one of embodiments 122-146, wherein a difference between a first log (water-octanol partition coefficient) the impurity and a second log (water-octanol partition coefficient) the product biomolecule is greater than about 2, 1.5, 1, 0.9, 0.8, 0.7, 0.6, 0.5, 0.4, 0.3, 0.2, 0.1, 0.09, 0.08, 0.07, 0.06, 0.05, 0.04, 0.03, 0.02, or 0.01. Embodiment 148. The method of any one of embodiments 122-147, wherein the impurity and the product biomolecule are hydrophobic. Embodiment 149. The method of any one of embodiments 122-148, wherein the impurity and the product biomolecule are hydrophilic. Embodiment 150. The method of any one of embodiments 122-149, wherein a difference between a first pKa of the impurity and a second pKa of the product biomolecule is at most 3, 2, 1, 0.9, 0.8, 0.7, 0.6, 0.5, 0.4, 0.3, 0.2, 0.1, 0.09, 0.08, 0.07, 0.06, 0.05, 0.04, 0.03, 0.02, or 0.01. Embodiment 151. The method of any one of embodiments 122-150, wherein a difference between a first pKa of the impurity and a second pKa of the product biomolecule is at least 3, 2, 1, 0.9, 0.8, 0.7, 0.6, 0.5, 0.4, 0.3, 0.2, 0.1, 0.09, 0.08, 0.07, 0.06, 0.05, 0.04, 0.03, 0.02, or 0.01. Embodiment 152. The method of any one of embodiments 122-151, wherein the impurity and the product biomolecule are stereoisomers of one another. Embodiment 153. The method of any one of embodiments 122-152, wherein the impurity and the product biomolecule comprise enantiomers of one another. Embodiment 154. The method of any one of embodiments 122-153, wherein a difference between a first mass-to-charge ratio of the impurity and a second mass-to-charge ratio of the product biomolecule is at most 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, or 100 kiloDaltons/e−. Embodiment 155. The method of any one of embodiments 122-154, wherein a difference between a first mass-to-charge ratio of the impurity and a second mass-to-charge ratio of the product biomolecule is at least 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, or 100 kiloDaltons/e−. Embodiment 156. The method of any one of embodiments 122-155, wherein the impurity comprises a first histidine tag and the product biomolecule comprises a second histidine tag. Embodiment 157. The method of embodiment 156, wherein the first histidine tag and the second histidine tag comprises the same number of amino acids. Embodiment 158. The method of any one of embodiments 122-157, wherein a difference between a first molecular weight of the impurity and a second molecular weight of the product biomolecule is at most 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, or 100 kiloDaltons. Embodiment 159. The method of any one of embodiments 122-158, wherein a difference between a first molecular weight of the impurity and a second molecular weight of the product biomolecule is at least 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, or 100 kiloDaltons. Embodiment 160. The method of any one of embodiments 122-159, further comprising purifying an impure biological preparation before (a) to generate the biological preparation, wherein the impure biological preparation and the biological preparation comprises the impurity. Embodiment 161. The method of embodiment 160, wherein the purifying increases the purity or the activity of the biological preparation compared to the impure biological preparation. Embodiment 162. The method of any one of embodiments 122-161, wherein the impurity comprises a lower abundance than the product biomolecule. Embodiment 163. The method of embodiment 162, wherein the impurity is at least 1, 2, 3, 4, 5, 6, 7, 8, 9, 10, 11, 12, 13, 14 or 15 times in magnitude less abundant than the product biomolecule by count, mass, or mass spectrometry signal intensity. Embodiment 164. The method of embodiment 162 or 163, wherein the impurity is at most 1, 2, 3, 4, 5, 6, 7, 8, 9, 10, 11, 12, 13, 14 or 15 times in magnitude less abundant than the product biomolecule by count, mass, or mass spectrometry signal intensity. Embodiment 165. The method of any one of embodiments 122-164, wherein the plurality of biomolecules comprises a reduced dynamic range on the one or more surfaces compared to a dynamic range of the plurality of biomolecule in the biological preparation. Embodiment 166. The method of embodiment 165, wherein the reduced dynamic range is at least 1, 2, 3, 4, 5, 6, 7, 8, 9, 10, 11, 12, 13, 14 or 15 times in magnitude less than the dynamic range. Embodiment 167. The method of embodiment 165 or 166, wherein the reduced dynamic range is at most 1, 2, 3, 4, 5, 6, 7, 8, 9, 10, 11, 12, 13, 14 or 15 times in magnitude less than the dynamic range. Embodiment 168. The method of any one of embodiments 122-167, wherein the biological preparation comprises a drug or a metabolite thereof. Embodiment 169. The method of embodiment 168, wherein the impurity comprises the drug. Embodiment 170. The method of embodiment 168, wherein the impurity comprises the metabolite thereof. Embodiment 171. The method of embodiment 168, wherein the product biomolecule comprises the drug. Embodiment 172. The method of embodiment 168, wherein the host cell protein comprises the drug. Embodiment 173. The method of any one of embodiments 168-172, wherein the drug comprises an antibody, a biotherapeutic, or a chemotherapeutic biomolecule. Embodiment 174. The method of any one of embodiments 168-173, wherein the drug comprises a methamphetamine, isotretinoin, an antibiotic, an anti-platelet medication, dutasteride, a blood thinner, insulin, hepatitis B immune globulin, a growth hormone, tamsulosin, finasteride, acitretin, etretinate, or any combination thereof. Embodiment 175. The method of any one of embodiments 168-174, wherein the impurity is capable of reducing the activity of the product biomolecule, wherein the impurity is produced by the host organism or a contaminating organism that is extraneous to the host organism. Embodiment 176. The method of any one of embodiments 122-167, wherein the biological preparation comprises a vaccine. Embodiment 177. The method of embodiment 176, wherein the product biomolecule comprises an antigen of a pathogenic bacteria, a virus, or a derivative thereof. Embodiment 178. The method of embodiment 176 or 177, wherein the product biomolecule comprises an immunoprotein or a derivative thereof. Embodiment 179. The method of any one of embodiments 122-167, wherein the product biomolecule comprises an enzyme. Embodiment 180. The method of embodiment 179, wherein the enzyme is configured to degrade a synthetic polymer, degrade an oil, or catalyze ethanol production. Embodiment 181. The method of any one of embodiments 122-167, wherein the biological preparation comprises blood, plasma, platelets, clotting factors, or any combination thereof. Embodiment 182. The method of any one of embodiments 122-167, wherein the impurity comprises a biomolecule produced by a pathogen. Embodiment 183. The method of embodiment 182, wherein the pathogen comprises a Hepatitis B virus, a Hepatitis C virus, a COVID-19, or a HIV. Embodiment 184. The method of any one of embodiments 122-167, wherein the biological preparation comprises a human consumable product or a livestock consumable product. Embodiment 185. The method of embodiment 184, wherein the human consumable product or the livestock consumable product comprises poultry, beef, pork, vegetables, fungi, a fermented product, or a meat substitute. Embodiment 186. The method of embodiment 184 or 185, wherein the impurity comprises a bacterial biomolecule. Embodiment 187. The method of any one of embodiments 184-186, further comprising estimating a shelf life of the biological preparation based on the deviation. Embodiment 188. The method of any one of embodiments 184-187, further comprising determining a fitness of the biological preparation for human or livestock consumption based on the deviation. Embodiment 189. The method of any one of embodiments 184-188, wherein the human consumable product comprises a fermented product. Embodiment 190. The method of embodiment 189, wherein the fermented product comprises ethanol, acetic acid, or lactic acid. Embodiment 191. The method of embodiment 189 or 190, wherein the fermented product comprises beer or wine. Embodiment 192. The method of embodiment 189 or 190, wherein the fermented product comprises vinegar. Embodiment 193. The method of embodiment 189 or 190, wherein the fermented product comprises yogurt. Embodiment 194. The method of any one of embodiments 168-193, wherein the impurity is capable of increasing the risk of complications when the biological preparation is administered to a subject. Embodiment 195. The method of any one of embodiments 168-193, wherein the impurity, when administered to a human subject or ingested by the human subject, is expected to reduce the activity/stability of the product biomolecule on the human subject. Embodiment 196. The method of any one of embodiments 168-193, wherein the impurity, when administered to a human subject or ingested by the human subject, is capable of harming the human subject Embodiment 197. The method of any one of embodiments 168-193, wherein the biological preparation, when comprising the host organism or the contaminating organism and administered to a human subject or ingested by the human subject, is capable of harming the human subject. Embodiment 198. The method of any one of embodiments 196 or 197, wherein the harming comprises: an immune response, a blood coagulation, an allergic reaction, a food poisoning, or any combination thereof. Embodiment 199. The method of any one of embodiments 122-198, wherein the deviation comprises a difference between a level of activity of the biological preparation and a reference level of activity for the reference composition. Embodiment 200. The method of embodiment 199, wherein the level of activity is assayed by contacting the plurality of biomolecules on the one or more surfaces with an activity assay. Embodiment 201. The method of embodiment 200, wherein the activity assay comprises an in vitro cell culture. Embodiment 202. The method of embodiment 200, wherein the activity assay comprises a substrate. Embodiment 203. The method of embodiment 200, wherein the activity assay comprises an immunoaffinity assay. Embodiment 204. The method of embodiment 200, wherein the activity assay comprises an avidity assay. Embodiment 205. The method of any one of embodiments 122-198, wherein the deviation comprises a difference between a level of safety of the biological preparation and a reference level of safety for the reference composition. Embodiment 206. The method of embodiment 205, wherein the level of safety is assayed by contacting the plurality of biomolecules on the one or more surfaces with a safety assay.

Embodiment 207. The method of embodiment 205 or 206, wherein the level of safety comprises a level of immunogenicity and the reference level of safety comprises a reference level of immunogenicity. Embodiment 208. The method of embodiment 207, wherein the level of immunogenicity is assayed by contacting the plurality of biomolecules on the one or more surfaces with one or more human blood samples or derivatives thereof. Embodiment 209. The method of embodiment 208, wherein the one or more human blood samples or derivatives thereof comprises one or more white blood cells. Embodiment 210. The method of any one of embodiments 122-209, wherein the deviation comprises a detectable level of the impurity. Embodiment 211. The method of any one of embodiments 122-210, wherein the deviation comprises a level of the product biomolecule that is below a reference level of the product biomolecule. Embodiment 212. The method of any one of embodiments 122-211, wherein the deviation comprises a difference between a level of the impurity and a reference level of the impurity. Embodiment 213. The method of any one of embodiments 122-212, further comprising purifying the biological preparation based on the deviation. Embodiment 214. The method of embodiment 213, wherein the purifying is performed when the difference is greater than 1, 2, 3, 4, 5, or 6 times a standard deviation of measurement for the level of the impurity. Embodiment 215. The method of embodiment 213 or 214, wherein the purifying is performed when the difference is greater than 1, 2, 3, 4, 5, or 6 times a standard error of measurement for the level of the impurity, wherein the standard error is based on at least N number of assays. Embodiment 216. The method of any one of embodiments 122-215, wherein the reference composition comprises at least about 1, 2, 3, 4, 5, 6, 7, 8, 9, 10, 20, 30, 40, 50, 60, 70, 80, 90, 91, 92, 93, 94, 95, 96, 97, 98, 99, 99.1, 99.2, 99.3, 99.4, 99.5, 99.6, 99.7, 99.8, 99.9, 99.9, 99.91, 99.92, 99.93, 99.94, 99.95, 99.96, 99.97, 99.98, 99.99, 99.99, 99.991, 99.992, 99.992, 99.993, 99.994, 99.995, 99.996, 99.997, 99.998, 99.999, 99.999, 99.9991, 99.9992, 99.9993, 99.9994, 99.9995, 99.9996, 99.9997, 99.9998, or 99.9999 percent purity of the product biomolecule. Embodiment 217. The method of embodiment any one of embodiments 122-216, wherein the reference composition comprises at most about 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 91, 92, 93, 94, 95, 96, 97, 98, 99, 99.1, 99.2, 99.3, 99.4, 99.5, 99.6, 99.7, 99.8, 99.9, 99.9 percent purity of the impurity. Embodiment 218. The method of embodiment 217, wherein the reference composition comprises at most about 1, 2, 3, 4, 5, 6, 7, 8, 9, 10, 20, 30, 40, 50, 60, 70, 80, 90, 100, 200, 300, 400, 500, 600, 700, 800, or 900 ppm of the impurity. Embodiment 219. The method of embodiment 218, wherein the reference composition comprises at most about 1, 2, 3, 4, 5, 6, 7, 8, 9, 10, 20, 30, 40, 50, 60, 70, 80, 90, 100, 200, 300, 400, 500, 600, 700, 800, or 900 ppb of the impurity. Embodiment 220. The method of any one of embodiments 216-219, wherein the percent purity is based on a signal intensity of the product determined from the assaying. Embodiment 221. The method of any one of embodiments 122-220, wherein the deviation is determined using a machine learning algorithm. Embodiment 222. The method of embodiment 221, wherein the machine learning algorithm is configured to receive one or more features that represent composition of the plurality of biomolecules and output the deviation based on the composition. Embodiment 223. The method of embodiment 221 or 222, wherein the machine learning algorithm is trained to learn the reference level of the impurity based on a first plurality of samples comprising the purity or the activity above a predetermined threshold, a second plurality of samples comprising the purity or the activity below the predetermined threshold, or both. Embodiment 224. The method of any one of embodiments 221-223, wherein the method further comprises, using the machine learning algorithm, classifying the biological preparation as comprising the purity or the activity above the predetermined threshold or below the predetermined threshold based on the composition of the plurality of biomolecule. Embodiment 225. The method of any one of embodiments 122-224, further comprising monitoring the biological preparation by assaying the plurality of biomolecules a plurality of times to determine a plurality of deviation at the plurality of times. Embodiment 226. The method of embodiment 225, further comprising determining a drift in the genetic makeup of the host organism based on the plurality of deviations.

Embodiment 227. The method of embodiment 225, further comprising determining a drift in the population of the cell culture based on the plurality of deviations. Embodiment 228. The method of embodiment 225, further comprising determining a contamination in a workflow for producing the biological preparation based on the plurality of deviations.

Embodiment 229. A method for detecting an impurity in a biological preparation, comprising: (a) contacting the biological preparation with the one or more surfaces to adsorb a plurality of biomolecules in the biological preparation onto the one or more surfaces; and (b) assaying the plurality of biomolecules to detect a biomolecule fingerprint of the biological preparation, wherein the biomolecule fingerprint comprises a signature of the impurity in the plurality of biomolecules.

Embodiment 230. An apparatus for characterizing biological preparation, comprising: a first chamber configured to hold the biological preparation, wherein the biological preparation comprises a plurality of biomolecules, wherein the plurality of biomolecules comprises a product biomolecule and an impurity; a second chamber comprising one or more surfaces; a loader operably coupled to the first chamber and the second chamber, wherein the loader is configured to transfer the biological preparation between the first chamber and the second chamber; and a computer readable medium for measuring a purity or an activity of the biological preparation comprising machine-executable code that, upon execution by a processor, implements a method comprising: contacting, using the loader, the biological preparation from the first chamber with the one or more surfaces in the second chamber to adsorb the plurality of biomolecules in the biological preparation onto the one or more surfaces.

Embodiment 231. The apparatus of embodiment 230, wherein the method further comprises assaying the plurality of biomolecules to determine a deviation between a composition of the plurality of biomolecules and a reference composition, wherein the deviation indicates the purity or the activity of the biological preparation. Embodiment 232. The apparatus of embodiment 230 or 231, further comprising a first separator operably coupled to the second chamber, wherein the first separator is configured to separate the plurality of biomolecules adsorbed on the one or more surfaces from the biological preparation. Embodiment 233. The apparatus of embodiment 232, wherein the first separator comprises a magnet. Embodiment 234. The apparatus of any one of embodiments 230-233, further comprising a second separator operably coupled to the second chamber, wherein the second separator is configured to separate the plurality of biomolecules adsorbed on the one or more surfaces from the one or more surfaces. Embodiment 235. The apparatus of any one of embodiments 230-234, further comprising one or more purifiers for purifying the biological preparation. Embodiment 236. The apparatus of embodiment 235, wherein the method further comprises purifying the biological preparation using the one or more purifiers based on the product quality. Embodiment 237. The apparatus of embodiment 235 or 236, wherein the one or more purifiers comprise a plurality of purifiers. Embodiment 238. The apparatus of embodiment 237, wherein the plurality of purifiers is provided in a sequence such that a purified biological preparation from one purifier in the plurality of purifiers feeds into another purifier in the plurality of purifiers. Embodiment 239. The apparatus of embodiment 238, wherein process conditions of the plurality of purifiers are based on the product quality. Embodiment 240. The apparatus of any one of embodiments 235-239, wherein the method further comprises using the one or more purifiers to purify the biological preparation based on the deviation to improve the purity or the activity of the biological preparation. Embodiment 241. The apparatus of any one of embodiments 230-240, further comprising a third chamber operably coupled to the loader, wherein the chamber comprises an in vitro cell culture or human blood samples or derivatives thereof. Embodiment 242. The apparatus of any one of embodiments 230-241, wherein the method further comprises certifying or rejecting the biological preparation based on the deviation. Embodiment 243. The apparatus of any one of embodiments 230-242, further comprising a mass spectrometer for performing the assaying to determine the deviation. Embodiment 244. The apparatus of any one of embodiments 230-243, wherein the first chamber comprises an incubator. Embodiment 245. The apparatus of embodiment 244, wherein the first chamber comprises a heater or a cooler operably connected to the incubator. Embodiment 246. The apparatus of embodiment 245, wherein the method comprises controlling the temperature of the first chamber using the heater or the cooler. Embodiment 247. The apparatus of any one of embodiments 244-246, wherein the first chamber is pressurized Embodiment 248. The apparatus of any one of embodiments 244-247, wherein the apparatus comprises a plurality of chamber comprising the first chamber, wherein each chamber in the plurality of chambers is operably connected to the loader and the second chamber, and wherein each chamber in the plurality of chambers comprises a portion of the biological preparation. Embodiment 249. The apparatus of any one of embodiments 244-248, wherein each chamber in the plurality of chambers comprises a different species or strain of a host organism configured to produce at least a portion of the biological preparation. Embodiment 250. The apparatus of any one of embodiments 230-249, further comprising one or more particles comprising the one or more surfaces and one or more supports. Embodiment 251. The apparatus of embodiment 250, wherein the one or more supports comprise a paramagnetic material. Embodiment 252. The apparatus of embodiment 251, wherein the paramagnetic material comprises a superparamagnetic material. Embodiment 253. The apparatus of any one of embodiments 230-252, wherein the one or more surfaces comprises a plurality of surface types. Embodiment 254. The apparatus of embodiment 253, wherein the one or more particles comprise a plurality of particles comprising the plurality of surface types. Embodiment 255. The apparatus of any one of embodiments 250-254, wherein the one or more particles comprise one or more microparticles. Embodiment 256. The apparatus of any one of embodiments 250-255, wherein the one or more particles comprise one or more nanoparticles. Embodiment 257. The apparatus of any one of embodiments 250-256, wherein the one or more particles comprise one or more porous particles. Embodiment 258. The apparatus of any one of embodiments 230-257, wherein the one or more surfaces are configured to reduce a dynamic range of the plurality of biomolecules in the biological preparation when the plurality of biomolecules is adsorbed on the one or more surfaces. Embodiment 259. The apparatus of any one of embodiments 230-258, wherein the impurity comprises a lower abundance than the product biomolecule. Embodiment 260. The apparatus of embodiment 259, wherein the impurity is at least 1, 2, 3, 4, 5, 6, 7, 8, 9, 10, 11, 12, 13, 14 or 15 times in magnitude less abundant than the product biomolecule by count, mass, or mass spectrometry signal intensity. Embodiment 261. The apparatus of embodiment 259 or 260, wherein the impurity is at most 1, 2, 3, 4, 5, 6, 7, 8, 9, 10, 11, 12, 13, 14 or 15 times in magnitude less abundant than the product biomolecule by count, mass, or mass spectrometry signal intensity. Embodiment 262. The apparatus of any one of embodiments 258-261, wherein the dynamic range of the plurality of biomolecules is reduced by at least 1, 2, 3, 4, 5, 6, 7, 8, 9, 10, 11, 12, 13, 14 or 15 times in magnitude. Embodiment 263. The apparatus of any one of embodiments 258-262, wherein the reduced dynamic range is at most 1, 2, 3, 4, 5, 6, 7, 8, 9, 10, 11, 12, 13, 14 or 15 times in magnitude less than the dynamic range.

Embodiment 264. An apparatus for detecting an impurity in a biological preparation, comprising: a plurality of chambers in operable connection with one another, wherein the plurality of chambers comprises one or more surfaces; one or more fluid transfer devices operably coupled to the plurality of chambers; and a computer readable medium for detecting the impurity in the biological preparation comprising machine-executable code that, upon execution by a processor, implements a method comprising: contacting, using the one or more fluid transfer devices, the biological preparation with the one or more surfaces to adsorb a plurality of biomolecules in the biological preparation onto the one or more surfaces.

Embodiment 265. The apparatus of embodiment 264, wherein the method further comprises assaying the plurality of biomolecules to detect a biomolecule fingerprint of the biological preparation, wherein the biomolecule fingerprint comprises a signature of the contaminating biomolecule in the plurality of biomolecules. Embodiment 266. The apparatus of embodiment 264 or 265, further comprising a first separator operably coupled to the plurality of chambers, wherein the first separator is configured to separate the plurality of biomolecules adsorbed on the one or more surfaces from the biological preparation. Embodiment 267. The apparatus of embodiment 266, wherein the first separator comprises a magnet. Embodiment 268. The apparatus of any one of embodiments 264-267, further comprising a second separator operably coupled to the second chamber, wherein the second separator is configured to separate the plurality of biomolecules adsorbed on the one or more surfaces from the one or more surfaces. Embodiment 269. The apparatus of any one of embodiments 264-268, further comprising one or more purifiers for purifying the biological preparation. Embodiment 270. The apparatus of embodiment 269, wherein the method further comprises purifying the biological preparation using the one or more purifiers based on the biomolecule fingerprint. Embodiment 271. The apparatus of embodiment 269 or 270, wherein the one or more purifiers comprise a plurality of purifiers. Embodiment 272. The apparatus of embodiment 271, wherein the plurality of purifiers is provided in a sequence such that a purified biological preparation from one purifier in the plurality of purifiers feeds into another purifier in the plurality of purifiers. Embodiment 273. The apparatus of embodiment 272, wherein process conditions of the plurality of purifiers are based on the biomolecule fingerprint. Embodiment 274. The apparatus of any one of embodiments 269-273, wherein the method further comprises using the one or more purifiers to purify the biological preparation based on the biomolecule fingerprint to reduce the signature of the contaminating biomolecule. Embodiment 275. The apparatus of any one of embodiments 264-274, further comprising a third chamber operably coupled to the loader, wherein the chamber comprises an in vitro cell culture or human blood samples or derivatives thereof. Embodiment 276. The apparatus of any one of embodiments 264-275, wherein the method further comprises certifying or rejecting the biological preparation based on the biomolecule fingerprint. Embodiment 277. The apparatus of any one of embodiments 264-276, further comprising a mass spectrometer for performing the assaying to determine the biomolecule fingerprint. Embodiment 278. The apparatus of any one of embodiments 264-277, wherein the plurality of chambers comprises an incubator. Embodiment 279. The apparatus of embodiment 278, wherein the plurality of chambers comprises a heater or a cooler operably connected to the incubator. Embodiment 280. The apparatus of embodiment 279, wherein the method comprises controlling the temperature the plurality of chambers using the heater or the cooler. Embodiment 281. The apparatus of any one of embodiments 278-280, wherein the plurality of chambers is pressurized Embodiment 282. The apparatus of any one of embodiments 264-281, wherein each chamber in the plurality of chambers comprises a different species or strain of a host organism configured to produce at least a portion of the biological preparation. Embodiment 283. The apparatus of any one of embodiments 264-282, further comprising one or more particles comprising the one or more surfaces and one or more supports. Embodiment 284. The apparatus of embodiment 283, wherein the one or more supports comprise a paramagnetic material. Embodiment 285. The apparatus of embodiment 284, wherein the paramagnetic material comprises a superparamagnetic material. Embodiment 286. The apparatus of any one of embodiments 264-285, wherein the one or more surfaces comprises a plurality of surface types. Embodiment 287. The apparatus of embodiment 286, wherein the one or more particles comprise a plurality of particles comprising the plurality of surface types. Embodiment 288. The apparatus of any one of embodiments 283-287, wherein the one or more particles comprise one or more microparticles. Embodiment 289. The apparatus of any one of embodiments 283-288, wherein the one or more particles comprise one or more nanoparticles. Embodiment 290. The apparatus of any one of embodiments 283-289, wherein the one or more particles comprise one or more porous particles. Embodiment 291. The apparatus of any one of embodiments 283-290, wherein the one or more surfaces are configured to reduce a dynamic range of the plurality of biomolecules in the biological preparation when the plurality of biomolecules is adsorbed on the one or more surfaces. Embodiment 292. The apparatus of any one of embodiments 264-291, wherein the contaminating biomolecule is at least 1, 2, 3, 4, 5, 6, 7, 8, 9, 10, 11, 12, 13, 14 or 15 times in magnitude less abundant than another biomolecule in the plurality of biomolecules by count, mass, or mass spectrometry signal intensity. Embodiment 293. The apparatus of any one of embodiments 264-292, wherein the contaminating biomolecule is at most 1, 2, 3, 4, 5, 6, 7, 8, 9, 10, 11, 12, 13, 14 or 15 times in magnitude less abundant than another biomolecule in the plurality of biomolecules by count, mass, or mass spectrometry signal intensity. Embodiment 294. The apparatus of any one of embodiments 264-293, wherein the dynamic range of the plurality of biomolecules is reduced by at least 1, 2, 3, 4, 5, 6, 7, 8, 9, 10, 11, 12, 13, 14 or 15 times in magnitude.

Embodiment 295. A computer-implemented method for generating a quality metric for a biological preparation, comprising: (a) receiving a plurality of mass spectrometry datasets for a plurality of polyamino acids in the biological preparation, wherein the plurality of polyamino acids comprises at least one product biomolecule and a plurality of impurities; (b) generating a plurality of polyamino acid identifications and a plurality of polyamino acid abundances for the plurality of polyamino acids based on the plurality of mass spectrometry datasets; and (c) processing the plurality of polyamino acid identifications to output the quality metric for the biological preparation.

Embodiment 296. The computer-implemented method of embodiment 295, wherein the plurality of polyamino acid abundances is indicative of relative abundances between the plurality of polyamino acids Embodiment 297. The computer-implemented method of embodiment 295 or 296, wherein the plurality of polyamino acid identifications comprise at least 1000, 2000, 3000, 4000, 5000, 6000, 7000, 8000, 9000, 10000, 15000, 20000, 25000, 30000, 35000, 40000, 45000, or 50000 polyamino acid identifications. Embodiment 298. The computer-implemented method of any one of embodiments 295-297, wherein the plurality of polyamino acids comprises a dynamic range of at least about 6, 7, 8, 9, 10, 11, 12, 13, 14, or 15 in the biological preparation. Embodiment 299. The computer-implemented method of any one of embodiments 295-298, wherein the plurality of polyamino acid abundances comprises a dynamic range less than about 6, 7, 8, 9, 10, 11, 12, 13, 14, or 15. Embodiment 300. The computer-implemented method of any one of embodiments 295-299, wherein the plurality of impurities comprises a proteoform of a polyamino acid in the plurality of polyamino acids. Embodiment 301. The computer-implemented method of embodiment 300, wherein the proteoform comprises a splicing variant, an allelic variant, or a post-translational modification variant. Embodiment 302. The computer-implemented method of embodiment 301, wherein the post-translational modification variant comprises a post-translational modification comprising: acylation, alkylation, prenylation, flavination, amination, deamination, carboxylation, decarboxylation, nitrosylation, halogenation, sulfurylation, glutathionylation, oxidation, oxygenation, reduction, ubiquitination, SUMOylation, neddylation, myristoylation, palmitoylation, isoprenylation, farnesylation, geranylgeranylation, glypiation, glycosylphosphatidylinositol anchor formation, lipoylation, heme functionalization, phosphorylation, phosphopantetheinylation, retinylidene Schiff base formation, diphthamide formation, ethanolamine phosphoglycerol functionalization, hypusine formation, beta-Lysine addition, acetylation, formylation, methylation, amidation, amide bond formation, butyrylation, gamma-carboxylation, glycosylation, polysialylation, malonylation, hydroxylation, iodination, nucleotide addition, phosphate ester formation, phosphoramidate formation, adenylation, uridylylation, propionylation, pyroglutamate formation, gluthathionylation, sulfenylation, sulfinylation, sulfonylation, succinylation, sulfation, glycation, carbonylation, isopeptide bond formation, biotinylation, carbamylation, oxidation, pegylation, citrullination, deamidation, eliminylation, disulfide bond formation, proteolytic cleavage, isoaspartate formation, racemization, protein splicing, chaperon-assisted folding, or any combination thereof. Embodiment 303. The computer-implemented method of any one of embodiments 295-302, wherein the processing comprises using a machine learning algorithm to score the biological preparation based on the plurality of polyamino acid identifications and the plurality of polyamino acid abundances to generate the quality metric. Embodiment 304. The computer-implemented method of embodiment 303, wherein the machine learning algorithm is trained using a first set of samples above a predetermined quality metric threshold, a second set of samples below the determined quality metric threshold, or both. Embodiment 305. The computer-implemented method of any one of embodiments 295-304, wherein the processing is based on a difference between the plurality of polyamino acid identifications and the plurality of polyamino acid abundances and a reference plurality of polyamino acid identifications and a reference plurality of polyamino acid abundances. Embodiment 306. The computer-implemented method of any one of embodiments 295-305, wherein the quality metric comprises a purity metric, an activity metric, a safety metric, or any combination thereof.

Embodiment 307. A computer-implemented system comprising: a digital processing device comprising: at least one processor, an operating system configured to perform executable instructions, a memory, and a computer program including instructions executable by the digital processing device to: (a) receive a plurality of mass spectrometry datasets obtained from a plurality of biological preparations; (b) processing the plurality of mass spectrometry datasets in real-time to generate a plurality of quality metrics for the plurality of biological preparations; and (c) providing process control instructions to a manufacturing process for producing the plurality of biological preparations.

Embodiment 308. The computer-implemented system of embodiment 307, wherein the process control instructions comprise certifying or rejecting one or more biological preparations in the plurality of biological preparations. Embodiment 309. The computer-implemented system of embodiment 307 or 308, wherein the process control instructions comprise providing change one or more process conditions of the manufacturing process. Embodiment 310. The computer-implemented system of any one of embodiments 307-309, wherein the processing is performed using one or more cloud-computing nodes. Embodiment 311. The computer-implemented system of any one of embodiments 307-310, wherein the processing is performed in less than 1, 2, 3, 4, 5, 6, 7, 8, 9, 10, 15, 30, 60, 120, 180, 240, 300, or 360 minutes. Embodiment 312. The computer-implemented system of any one of embodiments 307-311, wherein the plurality of biological preparations comprises a plurality of impurities.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the present disclosure may be employed in practicing the present disclosure. It is intended that the following claims define the scope of the present disclosure and that methods and structures within the scope of these claims and their equivalents be covered thereby.

What is claimed is:

1. A method for identifying one or more biomolecules, comprising:
(a) providing a supplemented medium;
(b) generating a set of biomolecules by incubating a cell in the supplemented medium under conditions sufficient for the cell to generate the set of biomolecules;
(c) contacting at least a portion of the supplemented medium with one or more surfaces to adsorb the set of biomolecules;
(d) removing the one or more surfaces and the set of biomolecules from the at least the portion of the supplemented medium to produce a separated sample;
(e) releasing the set of biomolecules from the one or more surfaces; and
(f) detecting at least a subset of the set of biomolecules, thereby identifying one or more biomolecules.

2. The method of claim 1, wherein, subsequent to the contacting of (c), the set of biomolecules comprises a reduced dynamic range when adsorbed on the one or more surfaces compared to an original dynamic range of the set of biomolecules in the supplemented medium.

3. The method of claim 1, wherein, in (a), the incubating the cell in the supplemented medium is performed for less than about 5 seconds, 5 minutes, 5 hours, or 5 days.

4. The method of claim 1, wherein the cell is afflicted with an infection or a mutation.

5. The method of claim 1, wherein the cell is a viable cell comprising a cancer cell, an epithelial cell, a bone cell, a muscle cell, a fat cell, a tissue cell, a senescent cell, a pluripotent cell, a stem cell, or a nerve cell.

6. The method of claim 1, wherein a particle comprises the surface.

7. The method of claim 6, wherein the particle is a nanoparticle.

8. The method of claim 1, wherein the contacting in (b) further comprises contacting the biological sample with a second surface to adsorb a second plurality of biomolecules onto the second surface.

9. The method of claim 1, wherein the detecting comprises mass spectrometry.

10. The method of claim 1, wherein the conditions sufficient for the cell to generate the set of biomolecules with the supplemented medium comprises a predetermined temperature, a predetermined pressure, a predetermined flow regime, a predetermined solvent environment, or a combination thereof.

11. The method of claim 10, wherein the conditions are kept constant.

12. The method of claim 1, wherein the supplemented medium comprises as a fraction or in whole a serum, a plasma, cerebral spinal fluid (CSF), synovial fluid (SF), urine, tears, crevicular fluid, semen, whole blood, milk, nipple aspirate, needle aspirate, ductal lavage, vaginal fluid, nasal fluid, ear fluid, gastric fluid, pancreatic fluid, trabecular fluid, lung lavage, prostatic fluid, sputum, fecal matter, bronchial lavage, fluid from swabbings, bronchial aspirants, sweat, saliva, or any combination thereof.

13. The method of claim 1, wherein the supplemented medium comprises a supernatant of a cell culture, a secretome of co-cultures, an exosome, a tissue or cell lysate, or any combination thereof.

14. The method of claim 1, wherein the supplemented medium comprises a synthetic supplemented medium.

15. The method of claim 1, wherein the set of biomolecules comprise one or more biomarkers, molecular signatures, secreted proteins, absorbed proteins, a secretome, an exosome, or any combination thereof.

16. The method of claim 1, further comprising repeating (a)-(e) for a second supplemented medium comprising a second set of biomolecules, wherein the second supplemented medium is generated by incubating the cell in the supplemented medium for a different length of time.

17. The method of claim 1, wherein the generating comprises release of an exosome or liposome by the cell.

18. The method of claim 1, wherein a biomolecule of the set of biomolecules is a polypeptide.

19. The method of claim 1, wherein the conditions sufficient for the cell to exchange the set of biomolecules with the supplemented medium comprises one or more of a presence or absence of an organic compound, a presence or absence of an inorganic compound, a presence or absence of an autocrine signaling molecule, a presence or absence of a paracrine signaling molecule, a presence or absence of an antigen, a presence or absence of one or more co-cultured cells, a presence or absence of radiation, a presence or absence of one or more toxins, a presence or absence of protein aggregates, a presence or absence of one or more proteins, a presence or absence of active viral particles, a presence or absence of inactivated viral particles, a presence or absence of applied heat or cooling, a presence or absence of applied mechanical stress, a presence or absence of electrical stimulation, a presence or absence of transposons, a presence or absence of exosomes, a presence or absence of liposomes, a presence or absence of coated nucleic acids, a presence or absence of shock, or any combination thereof.

20. The method of claim 19, wherein the conditions are varied over time.

21. The method of claim 1, further comprising determining one or more protein to protein interactions, biomarkers, molecular signatures, biomolecules absorbed by the cell, biomolecules secreted by the cell, biomolecules dissociating from cell surfaces, biomolecules being cleaved off or shed from the surface, macromolecular complexes budded, released of cleaved of the surface, biomolecules passively released by the cell, conventionally and unconventionally released proteins, apoptotic release of biomolecules, necrotic released biomolecules, post-translation modifications, cell to cell interactions, cell to cell communications, or any combination thereof based at least in part on the identification, wherein molecular signatures are any patterns of proteins/proteoforms indicative of a biological state.

22. The method of claim 1, further comprising a plurality of cells comprising the cell.

23. The method of claim 22, wherein the plurality of cells is comprised in a tissue, an organoid, an organism, or a plurality of organisms.

24. The method of claim 22, wherein the plurality of cells comprises at least a first cell of a first type and a second cell of a second type, such that the first cell exchanges one or more biomolecules of the set of biomolecules with the second cell.

25. The method of claim 24, wherein the first cell and the second cell are co-cultured.

26. The method of claim 24, wherein the first cell is comprised in a feeder culture for the second cell.

27. The method of claim 1, wherein the cell is comprised in a primary cell culture.

28. The method of claim 22, wherein the plurality of cells are disposed in a plurality of separate volumes, each volume comprising a different supplemented medium or incubating conditions.

29. The method of claim 1, wherein the releasing comprises use of a protease.

30. The method of claim 1, wherein the one or more surfaces comprise at least two surfaces comprising distinct physicochemical properties such that the at least two surfaces adsorb a different pattern of biomolecule abundance from the set of biomolecules.

31. The method of claim 1, further comprising determining the one or more biomolecules were generated by the cell and not originally present in the supplemented medium.

32. The method of claim 1, wherein the supplemented medium comprises fetal bovine serum.

33. The method of claim 1, wherein the set of biomolecules comprise a dynamic range of at least about 6, 7, 8, 9, 10, 11, 12, 13, 14, 15, 16, 17, 18, 19, or 20.

34. The method of claim 1, wherein the set of biomolecules comprises at least about 500, 600, 700, 800, 900, 1000, 2000, 3000, 4000, 5000, 6000, 7000, 8000, 9000, 10000, 20000, 30000, 40000, or 50000 biomolecules.

35. The method of claim 1, wherein a surface of the one or more surfaces is disposed on a magnetic substrate, and wherein the removing in (d) comprises using a magnetic field to separate the magnetic substrate from the at least a portion of the supplemented media.

36. A method for monitoring cell activity, comprising:
(a) incubating a cell such that the cell generates a biological sample comprising a plurality of biomolecules;
(b) contacting the biological sample with a surface to adsorb the plurality of biomolecules onto the surface;
(c) separating the surface from the biological sample;
(d) releasing at least a portion of the plurality of biomolecules on the surface;
(e) detecting the at least the portion of the plurality of biomolecules, thereby identifying the plurality of biomolecules; and
(f) repeating (a) through (d) after a predetermined amount of time, thereby monitoring an activity of the cell.

37. A method for identifying a low abundance biomolecule in a biological sample, comprising:
(a) incubating a cell in a predetermined environment, such that the cell produces a biological sample;
(b) contacting the biological sample with a surface to adsorb a plurality of low abundance biomolecules in the biological sample onto the surface;
(c) separating the surface from the biological sample;
(d) releasing at least a portion of the plurality of low abundance biomolecules on the surface; and
(e) detecting the at least the portion of the plurality of low abundance biomolecules, thereby identifying the plurality of low abundance biomolecules.

38. An apparatus for assaying a biological sample, comprising:
a substrate comprising a surface;
a cell culture chamber comprising a cell;
a loading unit that is operably coupled to the substrate and the cell culture chamber; and
a computer readable medium comprising machine-executable code that, upon execution by a processor, implements a method comprising:
i. providing a controlled environment to the cell in the cell culture chamber for a predetermined duration of time, such that the cell produces a biological sample in the cell culture chamber;
ii. transferring at least a portion of the biological sample from the cell culture chamber to the substrate using the loading unit, thereby contacting the at least the portion with the surface to adsorb a biomolecule in the at least the portion of the biological sample onto the surface; and
iii. assaying at least a portion of the biomolecule to detect the biomolecule in the biological sample.

39. A method for identifying one or more biomolecules, comprising:
(a) incubating a cell under conditions sufficient for the cell to generate an exosome, wherein the exosome comprises a plurality of biomolecules;
(b) contacting the exosome with one or more surfaces to capture at least a portion of the exosome;
(c) removing the one or more surfaces and the at least the portion of the exosome from the cell to produce a separated sample;
(d) releasing, in the separated sample, the at least the portion of the exosome from the one or more surfaces; and
(e) detecting at least a subset of the plurality of biomolecules in the at least the portion of the exosome, thereby identifying one or more biomolecules.

40. A method for monitoring cell activity, comprising:
(a) incubating a cell such that the cell generates a biological sample comprising an exosome, wherein the exosome comprises a plurality of biomolecules;
(b) contacting the biological sample with a surface to capture at least a portion of the exosome onto the surface;
(c) releasing the at least the portion of the exosome from the surface;
(d) detecting the at least the portion of the plurality of biomolecules in the at least the portion of the exosome, thereby identifying the plurality of biomolecules; and
(e) repeating (a) through (d) after a predetermined amount of time, thereby monitoring an activity of the cell.

41. A method for identifying a low abundance biomolecule in a biological sample, comprising:
(a) incubating a cell in a predetermined environment, such that the cell produces a biological sample comprising an exosome, wherein the exosome comprises a plurality of low-abundance biomolecules;
(b) contacting the biological sample with a surface to capture at least a portion of the exosome in the biological sample onto the surface;
(c) releasing the at least the portion of the exosome from the surface; and
(d) detecting at least the portion of the plurality of low abundance biomolecules in the at least the portion of the exosome, thereby identifying the plurality of low abundance biomolecules.

42. An apparatus for assaying a biological sample, comprising:
a substrate comprising a surface;
a cell culture chamber comprising a cell;
a loading unit that is operably coupled to the substrate and the cell culture chamber; and
a computer readable medium comprising machine-executable code that, upon execution by a processor, implements a method comprising:
i. providing a controlled environment to the cell in the cell culture chamber for a predetermined duration of time, such that the cell produces a biological sample comprising an exosome in the cell culture chamber, wherein the exosome comprises one or more biomolecules;
ii. transferring at least a portion of the biological sample from the cell culture chamber to the substrate using the loading unit, thereby contacting the at least the portion with the surface to adsorb the exosome in the at least the portion of the biological sample onto the surface; and
iii. assaying the exosome to detect the one or more biomolecules in the biological sample.

43. A method of identifying biomolecules, comprising:
(a) processing one or more exosomes to release a plurality of biomolecules in the one or more exosomes to an environment external to the one or more exosomes, wherein a subset of biomolecules in the plurality of biomolecules comprises a first distribution of relative abundances in the one or more exosomes;
(b) performing a composition improving assay on the plurality of biomolecules to increase the first distribution to a second distribution of relative abundances for the subset of biomolecules;
(c) assaying the plurality of biomolecules to identify the subset of biomolecules.

44. A method of identifying biomolecules, comprising:
(a) contacting a biological sample comprising one or more exosomes with a plurality of particles to non-specifically bind a subset of the one or more exosomes on the plurality of particles, wherein the plurality of particles comprises distinct physicochemical properties, and wherein the one or more exosomes comprise a plurality of biomolecules;
(b) processing the one or more exosomes to release the plurality of biomolecules to an environment external to the one or more exosomes; and
(c) assaying the plurality of biomolecules to identify at least a subset of biomolecules in the plurality of biomolecules.

45. A method for characterizing a biological preparation, comprising:
(a) contacting the biological preparation with one or more surfaces to adsorb a plurality of biomolecules in the biological preparation onto the one or more surfaces, wherein the plurality of biomolecules comprises a product biomolecule and an impurity; and
(b) assaying the plurality of biomolecules to determine a deviation between a composition of the plurality of biomolecules and a reference composition, wherein the deviation is indicative of a purity or an activity of the biological preparation.

46. A method for detecting an impurity in a biological preparation, comprising:
(a) contacting the biological preparation with the one or more surfaces to adsorb a plurality of biomolecules in the biological preparation onto the one or more surfaces; and
(b) assaying the plurality of biomolecules to detect a biomolecule fingerprint of the biological preparation, wherein the biomolecule fingerprint comprises a signature of the impurity in the plurality of biomolecules.

47. An apparatus for characterizing biological preparation, comprising:
a first chamber configured to hold the biological preparation, wherein the biological preparation comprises a plurality of biomolecules, wherein the plurality of biomolecules comprises a product biomolecule and an impurity;
a second chamber comprising one or more surfaces;
a loader operably coupled to the first chamber and the second chamber, wherein the loader is configured to transfer the biological preparation between the first chamber and the second chamber; and
a computer readable medium for measuring a purity or an activity of the biological preparation comprising machine-executable code that, upon execution by a processor, implements a method comprising: contacting, using the loader, the biological preparation from the first chamber with the one or more surfaces in the second chamber to adsorb the plurality of biomolecules in the biological preparation onto the one or more surfaces.

48. An apparatus for detecting an impurity in a biological preparation, comprising:
a plurality of chambers in operable connection with one another, wherein the plurality of chambers comprises one or more surfaces;
one or more fluid transfer devices operably coupled to the plurality of chambers; and
a computer readable medium for detecting the impurity in the biological preparation comprising machine-executable code that, upon execution by a processor, implements a method comprising: contacting, using the one or more fluid transfer devices, the biological preparation with the one or more surfaces to adsorb a plurality of biomolecules in the biological preparation onto the one or more surfaces.

49. A computer-implemented method for generating a quality metric for a biological preparation, comprising:
(a) receiving a plurality of mass spectrometry datasets for a plurality of polyamino acids in the biological preparation, wherein the plurality of polyamino acids comprises at least one product biomolecule and a plurality of impurities;
(b) generating a plurality of polyamino acid identifications and a plurality of polyamino acid abundances for the plurality of polyamino acids based on the plurality of mass spectrometry datasets; and
(c) processing the plurality of polyamino acid identifications to output the quality metric for the biological preparation.

50. A computer-implemented system comprising:
a digital processing device comprising:
at least one processor, an operating system configured to perform executable instructions, a memory, and a computer program including instructions executable by the digital processing device to:
(a) receive a plurality of mass spectrometry datasets obtained from a plurality of biological preparations;
(b) processing the plurality of mass spectrometry datasets in real-time to generate a plurality of quality metrics for the plurality of biological preparations; and
(c) providing process control instructions to a manufacturing process for producing the plurality of biological preparations.
